SEQUENCE LISTING (18 sequences)

SEQ ID NO: 1 - DNA, 2370 bp, Bacillus thuringiensis
atgaacaagaataatactaaattaagcacaagagccttaccaagttttattgattattttaatggcatttatggatttgccactggtatcaaagacattatgaacatgatttttaaaacggatacaggtggtgatctaaccctagacgaaattttaaagaatcagcagttactaaatgatatttctggtaaattggatggggtgaatggaagcttaaatgatcttatcgcacagggaaacttaaatacagaattatctaaggaaatattaaaaattgcaaatgaacaaaatcaagttttaaatgatgttaataacaaactcgatgcgataaatacgatgcttcgggtatatctacctaaaattacctctatgttgagtgatgtaatgaaacaaaattatgcgctaagtctgcaaatagaatacttaagtaaacaattgcaagagatttctgataagttggatattattaatgtaaatgtacttattaactctacacttactgaaattacacctgcgtatcaaaggattaaatatgtgaacgaaaaatttgaggaattaacttttgctacagaaactagttcaaaagtaaaaaaggatggctctcctgcagatattcttgatgagttaactgagttaactgaactagcgaaaagtgtaacaaaaaatgatgtggatggttttgaattttaccttaatacattccacgatgtaatggtaggaaataatttattcgggcgttcagctttaaaaactgcatcggaattaattactaaagaaaatgtgaaaacaagtggcagtgaggtcggaaatgtttataacttcttaattgtattaacagctctgcaagcaaaagcttttcttactttaacaacatgccgaaaattattaggcttagcagatattgattatacttctattatgaatgaacatttaaataaggaaaaagaggaatttagagtaaacatcctccctacactttctaatactttttctaatcctaattatgcaaaagttaaaggaagtgatgaagatgcaaagatgattgtggaagctaaaccaggacatgcattgattgggtttgaaattagtaatgattcaattacagtattaaaagtatatgaggctaagctaaaacaaaattatcaagtcgataaggattccttatcggaagttatttatggtgatatggataaattattgtgcccagatcaatctgaacaaatctattatacaaataacatagtatttccaaatgaatatgtaattactaaaattgatttcactaaaaaaatgaaaactttaagatatgaggtaacagcgaatttttatgattcttctacaggagaaattgacttaaataagaaaaaagtagaatcaagtgaagcggagtatagaacgttaagtgctaatgatgatggggtgtatatgccgttaggtgtcatcagtgaaacatttttgactccgattaatgggtttggcctccaagctgatgaaaattcaagattaattactttaacatgtaaatcatatttaagagaactactgctagcaacagacttaagcaataaagaaactaaattgatcgtcccgccaagtggttttattagcaatattgtagagaacgggtccatagaagaggacaatttagagccgtggaaagcaaataataagaatgcgtatgtagatcatacaggcggagtgaatggaactaaagctttatatgttcataaggacggaggaatttcacaatttattggagataagttaaaaccgaaaactgagtatgtaatccaatatactgttaaaggaaaaccttctattcatttaaaagatgaaaatactggatatattcattatgaagatacaaataataatttagaagattatcaaactattaataaacgttttactacaggaactgatttaaagggagtgtatttaattttaaaaagtcaaaatggagatgaagcttggggagataactttattattttggaaattagtccttctgaaaagttattaagtccagaattaattaatacaaataattggacgagtacgggatcaactaatattagcggtaatacactcactctttatcagggaggacgagggattctaaaacaaaaccttcaattagatagtttttcaacttatagagtgtatttttctgtgtccggagatgctaatgtaaggattagaaattctagggaagtgttatttgaaaaaagatatatgagcggtgctaaagatgtttctgaaatgttcactacaaaatttgagaaagataacttttatatagagctttctcaagggaataatttatatggtggtcctattgtacatttttacgatgtctctattaagtaa

SEQ ID NO: 2 - DNA, 2160 bp, Bacillus thuringiensis
atgaaactaaagaatcaagataagcatcaaagtttttctagcaatgcgaaagtagataaaatctctacggattcactaaaaaatgaaacagatatagaattacaaaacattaatcatgaagattgtttgaaaatgtctgagtatgaaaatgtagagccgtttgttagtgcatcaacaattcaaacaggtattggtattgcgggtaaaatacttggtaccctaggcgttccttttgcaggacaagtagctagtctttatagttttatcttaggtgagctatggcctaaggggaaaaatcaatgggaaatctttatggaacatgtagaagagattattaatcaaaaaatatcaacttatgcaagaaataaagcacttacagacttgaaaggattaggagatgccttagctgtctaccatgattcgcttgaaagttgggttggaaatcgtaataacacaagggctaggagtgttgtcaagagccaatatatcgcattagaattgatgttcgttcagaaactaccttcttttgcagtgtctggagaggaggtaccattattaccgatatatgcccaagctgcaaatttacatttgttgctattaagagatgcatctatttttggaaaagagtggggattatcatcttcagaaatttcaacattttataaccgtcaagtcgaacgagcaggagattattccgaccattgtgtgaaatggtatagcacaggtctaaataacttgaggggtacaaatgccgaaagttgggtacgatataatcaattccgtagagacatgactttaatggtactagatttagtggcactatttccaagctatgatacacaaatgtatccaattaaaactacagcccaacttacaagagaagtatatacagacgcaattgggacagtacatccgcatccaagttttacaagtacgacttggtataataataatgcaccttcgttctctgccatagaggctgctgttgttcgaaacccgcatctactcgattttctagaacaagttacaatttacagcttattaagtcgatggagtaacactcagtatatgaatatgtggggaggacataaactagaattccgaacaataggaggaacgttaaatatctcaacacaaggatctactaatacttctattaatcctgtaacattaccgttcacttctcgagacgtctataggactgaatcattggcagggctgaatctatttttaactcaacctgttaatggagtacctagggttgattttcattggaaattcgtcacacatccgatcgcatctgataatttctattatccagggtatgctggaattgggacgcaattacaggattcagaaaatgaattaccacctgaagcaacaggacagccaaattatgaatcttatagtcatagattatctcatataggactcatttcagcatcacatgtgaaagcattggtatattcttggacgcatcgtagtgcagatcgtacaaatacaattgagccaaatagcattacacaaataccattagtaaaagctttcaatctgtcttcaggtgccgctgtagtgagaggaccaggatttacaggtggggatatccttcgaagaacgaatactggtacatttggggatatacgagtaaatattaatccaccatttgcacaaagatatcgcgtgaggattcgctatgcttctaccacagatttacaattccatacgtcaattaacggtaaagctattaatcaaggtaatttttcagcaactatgaatagaggagaggacttagactataaaacctttagaactgtaggctttaccactccatttagctttttagatgtacaaagtacattcacaataggtgcttggaacttctcttcaggtaacgaagtttatatagatagaattgaatttgttccggtagaagtaacatatgaggcagaatatgattttgaaaaagcgcaagagaaggttactgcactgtttacatctacgaatccaagaggattaaaaacagatgtaaaggattatcatattgaccaggtatcaaatttagtagagtctctatcagatgaattctatcttgatgaaaagagagaattattcgagatagttaaatacgcgaagcaactccatattgagcgtaacatgtag

SEQ ID NO: 3 - DNA, 1902 bp, Bacillus thuringiensis
atgaatagtgtattgaatagcggaagaactactatttgtgatgcgtataatgtagcggctcatgatccatttagttttcaacacaaatcattagataccgtacaaaaggaatggacggagtggaaaaaaaataatcatagtttatacctagatcctattgttggaactgtggctagttttctgttaaagaaagtggggagtcttgttggaaaaaggatactaagtgagttacggaatttaatatttcctagtggtagtacaaatctaatgcaagatattttaagagagacagaaaaattcctgaatcaaagacttaatacagacactcttgcccgtgtaaatgcggaattgacagggctgcaagcaaatgtagaagagtttaatcgacaagtagataattttttgaaccctaaccgaaacgctgttcctttatcaataacttcttcagttaatacaatgcaacaattatttctaaatagattaccccagttccagatgcaaggataccaactgttattattacctttatttgcacaggcagccaatttacatctttcttttattagagatgttattctaaatgcagatgaatggggaatttcagcagcaacattacgtacgtatcgagattacttgaaaaattatacaagagattactctaactattgtataaatacgtatcaaagtgcgtttaaaggtttaaacactcgtttacacgatatgttagaatttagaacatatatgtttttaaatgtatttgagtatgtatctatctggtcgttgtttaaatatcaaagtcttctagtatcttccggtgctaatttatatgcaagtggtagtggaccacagcagacccaatcatttacttcacaagactggccatttttatattctcttttccaagttaattcaaattatgtgttaaatggatttagtggtgctaggctttctaataccttccctaatatagttggtttacctggttctactacaactcacgcattgcttgctgcaagggttaattacagtggaggaatttcgtctggtgatataggtgcatctccgtttaatcaaaattttaattgtagcacatttctccccccattgttaacgccatttgttaggagttggctagattcaggttcagatcgggagggcgttgccaccgttacaaattggcaaacagaatcctttgagacaactttagggttaaggagtggtgcttttacagctcgcggtaattcaaactatttcccagattattttattcgtaatatttctggagttcctttagttgttagaaatgaagatttaagaagaccgttacactataatgaaataagaaatatagcaagtccttcaggaacacctggtggagcacgagcttatatggtatctgtgcataacagaaaaaataatatccatgctgttcatgaaaatggttctatgattcatttagcgccaaatgactatacaggatttactatttcgccgatacatgcaactcaagtgaataatcaaacacgaacatttatttctgaaaaatttggaaatcaaggtgattctttaaggtttgaacaaaacaacacgacagctcgttatacgcttagagggaatggaaatagttacaatctttatttaagagtttcttcaataggaaattccactattcgagttactataaacggtagggtatatactgctacaaatgttaatactactacaaataacgatggagttaatgataatggagctcgtttttcagatattaatatcggtaatgtagtagcaagtagtaattctgatgtaccattagatataaatgtaacattaaactccggtactcaatttgatcttatgaatattatgcttgtaccaactaatatttcaccactttattaa

SEQ ID NO: 4 - DNA, 3531 bp, Bacillus thuringiensis
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctcttttggcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtgcgctggtacaatacgggattagagcgtgtatggggaccggattctagagattgggtaaggtataatcaatttagaagagagctaacacttactgtattagatatcgttgctctattctcaaattatgatagtcgaaggtatccaattcgaacagtttcccaattaacaagagaaatttatacgaacccagtattagaaaattttgatggtagttttcgtggaatggctcagagaatagaacagaatattaggcaaccacatcttatggatatccttaatagtataaccatttatactgatgtgcatagaggctttaattattggtcagggcatcaaataacagcttctcctgtagggttttcaggaccagaattcgcattccctttatttgggaatgcggggaatgcagctccacccgtacttgtctcattaactggtttggggatttttagaacattatcttcacctttatatagaagaattatacttggttcaggcccaaataatcaggaactgtttgtccttgatggaacggagttttcttttgcctccctaacgaccaacttgccttccactatatatagacaaaggggtacagtcgattcactagatgtaataccgccacaggataatagtgtaccacctcgtgcgggatttagccatcgattgagtcatgttacaatgctgagccaagcagctggagcagtttacaccttgagagctccaacgttttcttggcagcatcgcagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctactacaaatttacaattccatacatcaattgacggaagacctattaatcagggtaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatcagatgaattttgtctggatgaaaaacaagaattgtccgagaaagtcaaacatgcgaagcgacttagtgatgagcggaatttacttcaagatccaaacttcagagggatcaatagacaactagaccgtggctggagaggaagtacggatattaccatccaaggaggcgatgacgtattcaaagagaattacgttacgctattgggtacctttgatgagtgctatccaacgtatttatatcaaaaaatagatgagtcgaaattaaaagcctatacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagagaatccttgtgaatttaacagagggtatagggattacacgccactaccagttggttatgtgacaaaagaattagaatacttcccagaaaccgataaggtatggattgagattggagaaacggaaggaacatttatcgtggacagcgtggaattactccttatggaggaatag

SEQ ID NO: 5 - DNA, 3531 bp, Bacillus thuringiensis
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtgcgctggtacaatacgggattagagcgtgtatggggaccggattctagagattgggtaaggtataatcaatttagaagagagctaacacttactgtattagatatcgttgctctattctcaaattatgatagtcgaaggtatccaattcgaacagtttcccaattaacaagagaaatttatacgaacccagtattagaaaattttgatggtagttttcgtggaatggctcagagaatagaacagaatattaggcaaccacatcttatggatatccttaatagtataaccatttatactgatgtgcatagaggctttaattattggtcagggcatcaaataacagcttctcctgtagggttttcaggaccagaattcgcattccctttatttgggaatgcggggaatgcagctccacccgtacttgtctcattaactggtttggggatttttagaacattatcttcacctttatatagaagaattatacttggttcaggcccaaataatcaggaactgtttgtccttgatggaacggagttttcttttgcctccctaacgaccaacttgccttccactatatatagacaaaggggtacagtcgattcactagatgtaataccgccacaggataatagtgtaccacctcgtgcgggatttagccatcgattgagtcatgttacaatgctgagccaagcagctggagcagtttacaccttgagagctccaacgttttcttggcagcatcgcagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctactacaaatttacaattccatacatcaattgacggaagacctattaatcagggtaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatcagatgaattttgtctggatgaaaaacaagaattgtccgagaaagtcaaacatgcgaagcgacttagtgatgagcggaatttacttcaagatccaaacttcagagggatcaatagacaactagaccgtggctggagaggaagtacggatattaccatccaaggaggcgatgacgtattcaaagagaattacgttacgctattgggtacctttgatgagtgctatccaacgtatttatatcaaaaaatagatgagtcgaaattaaaagcctatacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagagaatccttgtgaatttaacagagggtatagggattacacgccactaccagttggttatgtgacaaaagaattagaatacttcccagaaaccgataaggtatggattgagattggagaaacggaaggaacatttatcgtggacagcgtggaattactccttatggaggaatag

SEQ ID NO: 6 - DNA, 3468 bp, Bacillus thuringiensis
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatctgatgaattttgtctggatgaaaaaaaagaattgtccgagaaagtcaaacatgcgaagcgacttagtgatgagcggaatttacttcaagatccaaactttagagggatcaatagacaactagaccgtggctggagaggaagtacggatattaccatccaaggaggcgatgacgtattcaaagagaattacgttacgctattgggtacctttgatgagtgctatccaacgtatttatatcaaaaaatagatgagtcgaaattaaaagcctatacccgttaccaattaagagggtatatcgaagatagtcaagacttagaaatctatttaattcgctacaatgccaaacacgaaacagtaaatgtgccaggtacgggttccttatggccgctttcagccccaagtccaatcggaaaatgtgcccatcattcccatcatttctccttggacattgatgttggatgtacagacttaaatgaggacttaggtgtatgggtgatattcaagattaagacgcaagatggccatgcaagactaggaaatctagaatttctcgaagagaaaccattagtaggagaagcactagctcgtgtgaaaagagcggagaaaaaatggagagacaaacgtgaaaaattggaatgggaaacaaatattgtttataaagaggcaaaagaatctgtagatgctttatttgtaaactctcaatatgatagattacaagcggataccaacatcgcgatgattcatgcggcagataaacgcgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaccgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgtgtagaagaggaagtatatccaaacaacacggtaacgtgtaatgattatactgcgactcaagaagaatatgagggtacgtacacttctcgtaatcgaggatatgacggagcctatgaaagcaattcttctgtaccagctgattatgcatcagcctatgaagaaaaagcatatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattacttcttatggaggaataa

SEQ ID NO: 7 - DNA, 3468 bp, Bacillus thuringiensis
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatctgatgaattttgtctggatgaaaaaaaagaattgtccgagaaagtcaaacatgcgaagcgacttagtgatgagcggaatttacttcaagatccaaactttagagggatcaatagacaactagaccgtggctggagaggaagtacggatattaccatccaaggaggcgatgacgtattcaaagagaattacgttacgctattgggtacctttgatgagtgctatccaacgtatttatatcaaaaaatagatgagtcgaaattaaaagcctatacccgttaccaattaagagggtatatcgaagatagtcaagacttagaaatctatttaattcgctacaatgccaaacacgaaacagtaaatgtgccaggtacgggttccttatggccgctttcagccccaagtccaatcggaaaatgtgcccatcattcccatcatttctccttggacattgatgttggatgtacagacttaaatgaggacttaggtgtatgggtgatattcaagattaagacgcaagatggccatgcaagactaggaaatctagaatttctcgaagagaaaccattagtaggagaagcactagctcgtgtgaaaagagcggagaaaaaatggagagacaaacgtgaaaaattggaatgggaaacaaatattgtttataaagaggcaaaagaatctgtagatgctttatttgtaaactctcaatatgatagattacaagcggatacgaatattgccatgattcatgcggcagataaacgcgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgtgtagaagaggaagtatatccaaacaacacggtaacgtgtaatgattatactgcgactcaagaagaatatgagggtacgtacacttctcgtaatcgaggatatgacggagcctatgaaagcaattcttctgtaccagctgattatgcatcagcctatgaagaaaaagcatatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattacttcttatggaggaataa

SEQ ID NO: 8 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 9 - protein, 719 aa, Bacillus thuringiensis
MKLKNQDKHQSFSSNAKVDKISTDSLKNETDIELQNINHEDCLKMSEYENVEPFVSASTIQTGIGIAGKILGTLGVPFAGQVASLYSFILGELWPKGKNQWEIFMEHVEEIINQKISTYARNKALTDLKGLGDALAVYHDSLESWVGNRNNTRARSVVKSQYIALELMFVQKLPSFAVSGEEVPLLPIYAQAANLHLLLLRDASIFGKEWGLSSSEISTFYNRQVERAGDYSDHCVKWYSTGLNNLRGTNAESWVRYNQFRRDMTLMVLDLVALFPSYDTQMYPIKTTAQLTREVYTDAIGTVHPHPSFTSTTWYNNNAPSFSAIEAAVVRNPHLLDFLEQVTIYSLLSRWSNTQYMNMWGGHKLEFRTIGGTLNISTQGSTNTSINPVTLPFTSRDVYRTESLAGLNLFLTQPVNGVPRVDFHWKFVTHPIASDNFYYPGYAGIGTQLQDSENELPPEATGQPNYESYSHRLSHIGLISASHVKALVYSWTHRSADRTNTIEPNSITQIPLVKAFNLSSGAAVVRGPGFTGGDILRRTNTGTFGDIRVNINPPFAQRYRVRIRYASTTDLQFHTSINGKAINQGNFSATMNRGEDLDYKTFRTVGFTTPFSFLDVQSTFTIGAWNFSSGNEVYIDRIEFVPVEVTYEAEYDFEKAQEKVTALFTSTNPRGLKTDVKDYHIDQVSNLVESLSDEFYLDEKRELFEIVKYAKQLHIERNM

SEQ ID NO: 10 - protein, 633 aa, Bacillus thuringiensis
MNSVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNISPLY

SEQ ID NO: 11 - protein, 1176 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLLAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFSNYDSRRYPIRTVSQLTREIYTNPVLENFDGSFRGMAQRIEQNIRQPHLMDILNSITIYTDVHRGFNYWSGHQITASPVGFSGPEFAFPLFGNAGNAAPPVLVSLTGLGIFRTLSSPLYRRIILGSGPNNQELFVLDGTEFSFASLTTNLPSTIYRQRGTVDSLDVIPPQDNSVPPRAGFSHRLSHVTMLSQAAGAVYTLRAPTFSWQHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKQELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 12 - protein, 1176 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFSNYDSRRYPIRTVSQLTREIYTNPVLENFDGSFRGMAQRIEQNIRQPHLMDILNSITIYTDVHRGFNYWSGHQITASPVGFSGPEFAFPLFGNAGNAAPPVLVSLTGLGIFRTLSSPLYRRIILGSGPNNQELFVLDGTEFSFASLTTNLPSTIYRQRGTVDSLDVIPPQDNSVPPRAGFSHRLSHVTMLSQAAGAVYTLRAPTFSWQHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKQELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 13 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 14 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 15 - protein, 1189 aa, Bacillus thuringiensis
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMESSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQKAVNALFTSSNQIGLKTDVTDYHIDQVSNLVDCLSDEFCLDEKRELSEKVKHAKRLSDERNLLQDPNFRGINRQPDRGWRGSTDITIQGGDDVFKENYVTLPGTVDECYPTYLYQKIDESKLKAYTRYELRGYIEDSQDLEIYLIRYNAKHEIVNVPGTGSLWPLSAQSPIGKCGEPNRCAPHLEWNPDLDCSCRDGEKCAHHSHHFTLDIDVGCTDLNEDLGVWVIFKIKTQDGHARLGNLEFLEEKPLLGEALARVKRAEKKWRDKREKLQLETNIVYKEAKESVDALFVNSQYDRLQVDTNIAMIHAADKRVHRIREAYLPELSVIPGVNAAIFEELEGRIFTAYSLYDARNVIKNGDFNNGLLCWNVKGHVDVEEQNNHRSVLVIPEWEAEVSQEVRVCPGRGYILRVTAYKEGYGEGCVTIHEIEDNTDELKFSNCVEEEVYPNNTVTCNNYTGTQEEYEGTYTSRNQGYDEAYGNNPSVPADYASVYEEKSYTDGRRENPCESNRGYGDYTPLPAGYVTKDLEYFPETDKVWIEIGETEGTFIVDSVELLLMEE

SEQ ID NO: 16 - DNA, 3570 bp, Bacillus thuringiensis
atggaggaaaataatcaaaatcaatgcataccttacaattgtttaagtaatcctgaagaagtacttttggatggagaacggatatcaactggtaattcatcaattgatatttctctgtcacttgttcagtttctggtatctaactttgtaccagggggaggatttttagttggattaatagattttgtatggggaatagttggcccttctcaatgggatgcatttctagtacaaattgaacaattaattaatgaaagaatagctgaatttgctaggaatgctgctattgctaatttagaaggattaggaaacaatttcaatatatatgtggaagcatttaaagaatgggaagaagatcctaataatccagcaaccaggaccagagtaattgatcgctttcgtatacttgatgggctacttgaaagggacattccttcgtttcgaatttctggatttgaagtaccccttttatccgtttatgctcaagcggccaatctgcatctagctatattaagagattctgtaatttttggagaaagatggggattgacaacgataaatgtcaatgaaaactataatagactaattaggcatattgatgaatatgctgatcactgtgcaaatacgtataatcggggattaaataatttaccgaaatctacgtatcaagattggataacatataatcgattacggagagacttaacattgactgtattagatatcgccgctttctttccaaactatgacaataggagatatccaattcagccagttggtcaactaacaagggaagtttatacggacccattaattaattttaatccacagttacagtctgtagctcaattacctacttttaacgttatggagagcagcgcaattagaaatcctcatttatttgatatattgaataatcttacaatctttacggattggtttagtgttggacgcaatttttattggggaggacatcgagtaatatctagccttataggaggtggtaacataacatctcctatatatggaagagaggcgaaccaggagcctccaagatcctttacttttaatggaccggtatttaggactttatcaaatcctactttacgattattacagcaaccttggccagcgccaccatttaatttacgtggtgttgaaggagtagaattttctacacctacaaatagctttacgtatcgaggaagaggtacggttgattctttaactgaattaccgcctgaggataatagtgtgccacctcgcgaaggatatagtcatcgtttatgtcatgcaacttttgttcaaagatctggaacaccttttttaacaactggtgtagtattttcttggacgcatcgtagtgcaactcttacaaatacaattgatccagagagaattaatcaaatacctttagtgaaaggatttagagtttgggggggcacctctgtcattacaggaccaggatttacaggaggggatatccttcgaagaaatacctttggtgattttgtatctctacaagtcaatattaattcaccaattacccaaagataccgtttaagatttcgttacgcttccagtagggatgcacgagttatagtattaacaggagcggcatccacaggagtgggaggccaagttagtgtaaatatgcctcttcagaaaactatggaaataggggagaacttaacatctagaacatttagatataccgattttagtaatcctttttcatttagagctaatccagatataattgggataagtgaacaacctctatttggtgcaggttctattagtagcggtgaactttatatagataaaattgaaattattctagcagatgcaacatttgaagcagaatctgatttagaaagagcacaaaaggcggtgaatgccctgtttacttcttccaatcaaatcgggttaaaaaccgatgtgacggattatcatattgatcaagtatccaatttagtggattgtttatcagatgaattttgtctggatgaaaagcgagaattgtccgagaaagtcaaacatgcgaagcgactcagtgatgagcggaatttacttcaagatccaaacttcagagggatcaatagacaaccagaccgtggctggagaggaagtacagatattaccatccaaggaggagatgacgtattcaaagagaattacgtcacactaccgggtaccgttgatgagtgctatccaacgtatttatatcagaaaatagatgagtcgaaattaaaagcttatacccgttatgaattaagagggtatatcgaagatagtcaagacttagaaatctatttgatccgttacaatgcaaaacacgaaatagtaaatgtgccaggcacgggttccttatggccgctttcagcccaaagtccaatcggaaagtgtggagaaccgaatcgatgcgcgccacaccttgaatggaatcctgatctagattgttcctgcagagacggggaaaaatgtgcacatcattcccatcatttcaccttggatattgatgttggatgtacagacttaaatgaggacttaggtgtatgggtgatattcaagattaagacgcaagatggccatgcaagactagggaatctagagtttctcgaagagaaaccattattaggggaagcactagctcgtgtgaaaagagcggagaagaagtggagagacaaacgagagaaactgcagttggaaacaaatattgtttataaagaggcaaaagaatctgtagatgctttatttgtaaactctcaatatgatagattacaagtggatacgaacatcgcaatgattcatgcggcagataaacgcgttcatagaatccgggaagcgtatctgccagagttgtctgtgattccaggtgtcaatgcggccattttcgaagaattagagggacgtatttttacagcgtattccttatatgatgcgagaaatgtcattaaaaatggcgatttcaataatggcttattatgctggaacgtgaaaggtcatgtagatgtagaagagcaaaacaaccaccgttcggtccttgttatcccagaatgggaggcagaagtgtcacaagaggttcgtgtctgtccaggtcgtggctatatccttcgtgtcacagcatataaagagggatatggagagggctgcgtaacgatccatgagatcgaagacaatacagacgaactgaaattcagcaactgtgtagaagaggaagtatatccaaacaacacagtaacgtgtaataattatactgggactcaagaagaatatgagggtacgtacacttctcgtaatcaaggatatgacgaagcctatggtaataacccttccgtaccagctgattacgcttcagtctatgaagaaaaatcgtatacagatggacgaagagagaatccttgtgaatctaacagaggctatggggattacacaccactaccggctggttatgtaacaaaggatttagagtacttcccagagaccgataaggtatggattgagatcggagaaacagaaggaacattcatcgtggatagcgtggaattactccttatggaggaataa

SEQ ID NO: 17 - protein, 1165 aa, Bacillus thuringiensis
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAASYPTFSAAESAIIRSPHLVDFLNSFTIYTDSLARYAYWGGHLVNSFRTGTTTNLIRSPLYGREGNTERPVTITASPSVPIFRTLSYITGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATFEAEYDLERAQKVVNALFTSTNQLGLKTDVTDYHIDQVSNLVACLSDEFCLDEKRELSEKVKHAKRLSDERNLLQDPNFRGINRQPDRGWRGSTDITIQGGDDVFKENYVTLPGTFDECYPTYLYQKIDESKLKAYTRYQLRGYIEDSQDLEIYLIRYNAKHEIVNVPGTGSLWPLSVENQIGPCGEPNRCAPHLEWNPDLHCSCRDGEKCAHHSHHFSLDIDVGCTDLNEDLGVWVIFKIKTQDGHARLGNLEFLEEKPLLGEALARVKRAEKKWRDKRETLQLETTIVYKEAKESVDALFVNSQYDRLQADTNIAMIHAADKRVHRIREAYLPELSVIPGVNAAIFEELEERIFTAFSLYDARNIIKNGDFNNGLLCWNVKGHVEVEEQNNHRSVLVIPEWEAEVSQEVRVCPGRGYILRVTAYKEGYGEGCVTIHEIENNTDELKFNNCVEEEVYPNNTVTCINYTATQEEYEGTYTSRNRGYDEAYGNNPSVPADYASVYEEKSYTDRRRENPCESNRGYGDYTPLPAGYVTKELEYFPETDKVWIEIGETEGTFIVDSVELLLMEE

SEQ ID NO: 18 - DNA, 3498 bp, Bacillus thuringiensis
atggaaataaataatcaaaaccaatgtgtgccttacaattgtttaagtaatcctaaggagataatattaggcgaggaaaggctagaaacagggaatactgtagcagacatttcattagggcttattaattttctatattctaattttgtaccaggaggaggatttatagtaggtttactagaattaatatggggatttatagggccttcgcaatgggatatttttttagctcaaattgagcaattgattagtcaaagaatagaagaatttgctaggaatcaggcaatttcaagattggaggggctaagcaatctttataaggtctatgttagagcgtttagcgactgggagaaagatcctactaatcctgctttaagggaagaaatgcgtatacaatttaatgacatgaatagtgctctcataacggctattccactttttagagttcaaaattatgaagttgctcttttatctgtatatgttcaagccgcaaacttacatttatctattttaagggatgtttcagttttcggagaaagatggggatatgatacagcgactatcaataatcgctatagtgatctgactagccttattcatgtttatactaaccattgtgtggatacgtataatcagggattaaggcgtttggaaggtcgttttcttagcgattggattgtatataatcgtttccggagacaattgacaatttcagtattagatattgttgcgttttttccaaattatgatattagaacatatccaattcaaacagctactcagctaacgagggaagtctatctggatttaccttttattaatgaaaatctttctcctgcagcaagctatccaaccttttcagctgctgaaagtgctataattagaagtcctcatttagtagactttttaaatagctttaccatttatacagatagtctggcacgttatgcatattggggagggcacttggtaaattctttccgcacaggaaccactactaatttgataagatcccctttatatggaagggaaggaaatacagagcgccccgtaactattaccgcatcacctagcgtaccaatatttagaacactttcatatattacaggccttgacaattcaaatcctgtagctggaatcgagggagtggaattccaaaatactataagtagaagtatctatcgtaaaagcggtccaatagattcttttagtgaattaccacctcaagatgccagcgtatctcctgcaattgggtatagtcaccgtttatgccatgcaacatttttagaacggattagtggaccaagaatagcaggcaccgtattttcttggacacaccgtagtgccagccctactaatgaagtaagtccatctagaattacacaaattccatgggtaaaggcgcatactcttgcatctggtgcctccgtcattaaaggtcctggatttacaggtggagatattctgactaggaatagtatgggcgagctggggaccttacgagtaaccttcacaggaagattaccacaaagttattatatacgtttccgttatgcttcggtagcaaataggagtggtacatttagatattcacagccaccttcgtatggaatttcatttccaaaaactatggacgcaggtgaaccactaacatctcgttcgttcgctcatacaacactcttcactccaataaccttttcacgagctcaagaagaatttgatctatacatccaatcgggtgtttatatagatcgaattgaatttataccggttactgcaacatttgaggcagaatatgatttagaaagagcgcaaaaggtggtgaatgccctgtttacgtctacaaaccaactagggctaaaaacagatgtgacggattatcatattgatcaggtatccaatctagttgcgtgtttatcggatgaattttgtctggatgaaaagagagaattgtccgagaaagttaaacatgcaaagcgactcagtgatgagcggaatttacttcaagatccaaacttcagagggatcaataggcaaccagaccgtggctggagaggaagtacggatattactatccaaggaggagatgacgtattcaaagagaattacgttacgctaccgggtacctttgatgagtgctatccaacgtatttatatcaaaaaatagatgagtcgaaattaaaagcctatacccgttatcaattaagagggtatatcgaagatagtcaagacttagaaatctatttaattcgttacaatgcaaaacacgaaatagtaaatgtaccaggtacaggaagtttatggcctctttctgtagaaaatcaaattggaccttgtggagaaccgaatcgatgcgcgccacaccttgaatggaatcctgatttacactgttcctgcagagacggggaaaaatgtgcacatcattctcatcatttctctttggacattgatgttggatgtacagacttaaatgaggacttaggtgtatgggtgatattcaagattaagacgcaagatggccacgcacgactagggaatctagagtttctcgaagagaaaccattattaggagaagcactagctcgtgtgaaaagagcggagaaaaaatggagagacaaacgcgaaacattacaattggaaacaactatcgtttataaagaggcaaaagaatctgtagatgctttatttgtaaactctcaatatgatagattacaagcggatacgaacatcgcgatgattcatgcggcagataaacgcgttcatagaattcgagaagcgtatctgccggagctgtctgtgattccgggtgtcaatgcggctatttttgaagaattagaagagcgtattttcactgcattttccctatatgatgcgagaaatattattaaaaatggcgatttcaataatggcttattatgctggaacgtgaaagggcatgtagaggtagaagaacaaaacaatcaccgttcagtcctggttatcccagaatgggaggcagaagtgtcacaagaggttcgtgtctgtccaggtcgtggctatatccttcgtgttacagcgtacaaagagggatatggagaaggttgcgtaacgatccatgagatcgagaacaatacagacgaactgaaattcaacaactgtgtagaagaggaagtatatccaaacaacacggtaacgtgtattaattatactgcgactcaagaagaatatgagggtacgtacacttctcgtaatcgaggatatgacgaagcctatggtaataacccttccgtaccagctgattatgcgtcagtctatgaagaaaaatcgtatacagatagacgaagagagaatccttgtgaatctaacagaggatatggagattacacaccactaccagctggttatgtaacaaaggaattagagtacttcccagagaccgataaggtatggattgagattggagaaacagaaggaacattcatcgtggacagcgtggaattactccttatggaggaatag

(19) United States
(12) Patent Application Publication
(10) Pub. No.: US 2022/0007651 A1
(43) Pub. Date: Jan. 13, 2022

(54) BACILLUS THURINGIENSIS STRAINS AND METHODS FOR CONTROLLING PESTS

(71) Applicant: Bayer CropScience LP, St. Louis, MO (US)

(72) Inventors: Lorena FERNANDEZ, Davis, CA (US); Punita JUNEJA, Davis, CA (US); Reed Nathan ROYALTY, Kirkwood, MO (US); Bjorn A. TRAAG, Walnut Creek, CA (US); Evelien Van Ekert, Pittsboro, NC (US); Emily L. WHITSON-WHENNEN, West Sacramento, CA (US)

(21) Appl. No.: 17/294,032

(22) PCT Filed: Nov. 14, 2019
(86) PCT No.: PCT/US2019/061554

§ 371 Date: May 14, 2021

Related U.S. Application Data
(60) Provisional application No. 62/767,040, filed on Nov. 14, 2018.

(51) Int. Cl. A01N 63/23; C12N 1/20

(52) U.S. Cl. CPC A01N 63/23; C12R 2001/075; C12N 1/205

(57) ABSTRACT

The present invention provides a composition comprising a biologically pure culture of a Bacillus thuringiensis strain or a cell-free preparation thereof comprising zwittermicin A, Vip3Aa11, Cry1Aa11, and Cry1Ab1. The present invention also relates to a method of controlling animal pests and protecting a useful plant or a part of a useful plant in need of protection from animal pest damage, the method comprising applying to an animal pest or an environment thereof an effective amount of a composition comprising a biologically pure culture of a Bacillus thuringiensis strain or a cell-free preparation thereof.

### CROSS-REFERENCE TO RELATED APPLICATIONS

This application claims priority to U.S. Provisional Patent Application No. 62/767,040, which was filed on Nov. 14, 2018, the entire contents of which are incorporated herein by reference in its entirety.

### REFERENCE TO SEQUENCE LISTING SUBMITTED ELECTRONICALLY

The official copy of the sequence listing is submitted electronically via EFS-Web as an ASCII-formatted sequence listing with a file named “BCS189005_WO_ST25.txt” created on Nov. 14, 2019, and having a size of 114 kilobytes, and is filed concurrently with the specification. The sequence listing contained in this ASCII-formatted document is part of the specification and is herein incorporated by reference in its entirety.

### TECHNICAL FIELD

The present invention relates to the field of bacterial strains and their ability to control animal pests. In particular, the present invention is directed to Bacillus thuringiensis strains with relatively high levels of insecticidal activity.

### BACKGROUND

Synthetic pesticides may be non-specific and therefore can act on organisms other than the target ones, including other naturally occurring beneficial organisms. Because of their chemical nature, they may also be toxic and non-biodegradable. Consumers worldwide are increasingly conscious of the potential environmental and health problems associated with the residuals of chemicals, particularly in food products. This has resulted in growing consumer pressure to reduce the use or at least the quantity of chemical (i.e., synthetic) pesticides. Thus, there is a need to manage food chain requirements while still allowing effective pest control.

A further problem arising with the use of synthetic insecticides is that the repeated and exclusive application of an insecticide often leads to selection of resistant insects. Normally, such insects are also cross-resistant against other active ingredients having the same mode of action. An effective control of the insects with said active compounds is then not possible any longer. However, active ingredients having new mechanisms of action are difficult and expensive to develop.

The risk of resistance development in insect populations as well as environmental and human health concerns have fostered interest in identifying alternatives to synthetic insecticides for managing plant and crop damage from insects. The use of biological control agents is one alternative.

Bacillus thuringiensis (Bt) is a Gram-positive spore forming soil bacterium characterized by its ability to produce crystalline inclusions that are specifically toxic to certain orders and species of plant pests, including insects, but are harmless to plants and other non-target organisms. For this reason, compositions comprising Bacillus thuringiensis strains or their insecticidal proteins can be used as environmentally acceptable insecticides to control agricultural insect pests or insect vectors of a variety of human or animal diseases.

There is a need for effective biological control agents with insecticidal activity to complement the use of traditional, synthetic insecticides and to address the growing challenge of insect resistance.

### SUMMARY

The present invention is directed to a composition comprising a biologically pure culture of a Bacillus thuringiensis strain or a cell-free preparation thereof comprising zwittermicin A, Vip3Aa11, Cry1Ia2, Cry2Ab1, Cry1Aa11, and Cry1Ab1; wherein Vip3Aa11 is expressed from a gene comprising a DNA sequence exhibiting at least 99.9% sequence identity to SEQ ID NO: 1; Cry1Ia2 is expressed from a gene comprising a DNA sequence exhibiting at least 99.9% sequence identity to SEQ ID NO: 2; Cry2Ab1 is expressed from a gene comprising a DNA sequence exhibiting at least 99.9% sequence identity to SEQ ID NO: 3; Cry1Aa11 is expressed from a gene comprising a DNA sequence exhibiting at least 99.9% sequence identity to SEQ ID NO: 4; Cry1Ab1 is expressed from a gene comprising a DNA sequence exhibiting at least 99.9% sequence identity to SEQ ID NO: 6; and expression of zwittermicin A with Vip3Aa11, Cry1Ia2, Cry2Ab1, Cry1Aa11, and/or Cry1Ab1 results in a synergistic insecticidal effect.

The present invention is directed to a composition comprising a biologically pure culture of a Bacillus thuringiensis strain or a cell-free preparation thereof comprising zwittermicin A, Vip3Aa, Cry1Aa, and Cry1Ab, wherein expression of zwittermicin A and Vip3Aa, Cry1Aa, and/or Cry1Ab results in a synergistic insecticidal effect. In one embodiment, the culture or cell-free preparation thereof comprises zwittermicin A, Vip3Aa, Cry1Aa, and Cry1Ab; wherein Vip3Aa is expressed from a gene comprising a DNA sequence exhibiting at least 99.9% sequence identity to SEQ ID NO: 1; Cry1Aa is expressed from a gene comprising a DNA sequence exhibiting at least 99.9% sequence identity to SEQ ID NO: 4 or SEQ ID NO: 5; and Cry1Ab is expressed from a gene comprising a DNA sequence exhibiting at least 99.9% sequence identity to SEQ ID NO: 6; and expression of zwittermicin A with Vip3Aa, Cry1Aa, and/or Cry1Ab results in a synergistic insecticidal effect.

The present invention is also directed to a biologically pure culture of a Bacillus thuringiensis strain or a cell-free preparation thereof comprising zwittermycin A, Cry1Ca and Cry1Da. In one embodiment, the pure culture or cell-free preparation thereof further comprises Vip3Aa, Cry1Aa, and Cry1Ab. In one embodiment, the culture or cell-free preparation thereof comprises zwittermicin A, Cry1Ca and Cry1Da, wherein the Cry1Ca is expressed from a gene comprising a DNA sequence exhibiting at least 99.9% sequence identity to SEQ ID NO: 16 and Cry1Da is expressed from a gene comprising a DNA sequence exhibiting at least 99.9% sequence identity to SEQ ID NO: 18. In another embodiment, the culture or cell-free preparation thereof comprises zwittermicin A, Vip3Aa, Cry1Aa, Cry1Ab1, Cry1Ca and Cry1Da, wherein Vip3Aa is expressed from a gene comprising a DNA sequence exhibiting at least 99.9% sequence identity to SEQ ID NO: 1; Cry1Aa is expressed from a gene comprising a DNA sequence exhibiting at least 99.9% sequence identity to SEQ ID NO: 4; Cry1Ab is expressed from a gene comprising a DNA sequence exhibiting at least 99.9% sequence identity to SEQ ID NO: 6; Cry1Ca is expressed from a gene comprising a DNA sequence exhibiting at least 99.9% sequence identity to SEQ ID NO: 16; and Cry1Da is expressed from a gene comprising a DNA sequence exhibiting at least 99.9% sequence identity to SEQ ID NO: 18, and expression of zwittermicin A with Vip3Aa, Cry1Aa, Cry1Ab, Cry1Ca and/or Cry1Da results in a synergistic insecticidal effect. In on aspect the expressed proteins are Vip3Aa11; Cry1Aa11, Cry1Aa8, Cry1Aa3 or Cry1Aa12; Cry1Ab1; Cry1Ca1 or Cry1Ca8; and/or Cry1Da1. In yet another aspect the Cry1Aa protein is Cry1Aa3. In yet another aspect the Cry1Ca protein is Cry1Ca8.

In one embodiment of any of the above compositions, the zwittermicin A is present in an amount at least 25-fold greater than that in a biologically pure culture of Bacillus thuringiensis subsp. kurstaki strain EG7841. In another embodiment, the zwittermicin A is present in an amount at least 5-fold greater than that in a biologically pure culture of Bacillus thuringiensis subsp. aizawai strain ABTS-1857.

In certain aspects, the synergistic insecticidal effect results in increased developmental delay and/or mortality. In one aspect, the synergistic insecticidal effect occurs with Spodoptera exigua, Plutella xylostella, and/or Trichoplusia ni.

In yet other embodiments, the Bacillus thuringiensis strain is Bacillus thuringiensis strain NRRL B-67685, Bacillus thuringiensis strain NRRL B-67687, Bacillus thuringiensis strain NRRL B-67688, or an insecticidal mutant thereof having all the identifying characteristics of the respective strain.

In one embodiment, the composition comprises a biologically pure culture of or a fermentation product of Bacillus thuringiensis strain NRRL B-67685, Bacillus thuringiensis strain NRRL B-67687, Bacillus thuringiensis strain NRRL B-67688, or an insecticidal mutant thereof having all the identifying characteristics of the respective strain.

In some embodiments, the insecticidal mutant strain has a genomic sequence with greater than about 90% sequence identity to Bacillus thuringiensis strain NRRL B-67685, Bacillus thuringiensis strain NRRL B-67687, or Bacillus thuringiensis strain NRRL B-67688.

In certain aspects, the composition further comprises an agriculturally acceptable carrier, inert, stabilization agent, preservative, nutrient, and/or physical property modifying agent. In one aspect, the composition is a liquid formulation or a dry formulation. In another aspect, the composition is a liquid formulation and comprises at least about 1×10⁴ colony forming units (CFU) of the Bacillus thuringiensis strain/mL.

In some embodiments, the present invention provides a composition comprising a) zwittermicin A and b) a Vip3A protein in a synergistically effective amount. In one embodiment, the Vip3A protein comprises an amino acid sequence exhibiting at least 90% sequence identity to SEQ ID NO: 8. In one aspect, the Vip3A protein is present in a biologically pure culture of an E. coli strain engineered to express Vip3A or a cell-free preparation thereof. In another aspect the Vip3A plus zwittermicin composition further comprises an agriculturally acceptable carrier, inert, stabilization agent, preservative, nutrient, and/or physical property modifying agent.

The present invention also relates to a method of controlling an animal pest, comprising applying to the animal pest or an environment thereof an effective amount of any of the s compositions disclosed herein.

In another embodiment, the present invention provides a method of protecting a useful plant or a part of a useful plant in need of protection from animal pest damage, the method comprising contacting an animal pest, a plant, a plant propagule, a seed of a plant, and/or a locus where a plant is growing or is intended to grow with an effective amount of any of the compositions disclosed herein.

In certain aspects, the composition is applied at about 1×10⁴ to about 1×10¹⁴ CFU per hectare or at about 0.1 kg to about 20 kg fermentation solids per hectare.

In some embodiments, the animal pest is from the order of Lepidoptera and is Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Cheimatobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, or Trichoplusia spp. In one embodiment, the animal pest is Spodoptera exigua, Plutella xylostella, or Trichoplusia ni.

In one aspect, the useful plant is selected from the group consisting of soybean, corn, wheat, triticale, barley, oat, rye, rape, millet, rice, sunflower, cotton, sugar beet, pome fruit, stone fruit, citrus, banana, strawberry, blueberry, almond, grape, mango, papaya, peanut, potato, tomato, pepper, cucurbit, cucumber, melon, watermelon, garlic, onion, broccoli, carrot, cabbage, bean, dry bean, canola, pea, lentil, alfalfa, trefoil, clover, flax, elephant grass, grass, lettuce, sugarcane, tea, tobacco and coffee; each in its natural or genetically modified form.

In one embodiment, the present invention provides the use of a composition as disclosed herein for controlling animal pests. In another embodiment, the present invention relates to the use of a composition as disclosed herein for protecting a useful plant or a part of a useful plant in need of protection from animal pest damage.

### BRIEF DESCRIPTION OF THE DRAWINGS

FIG. 1 depicts the relative levels of zwittermicin A in thirty-nine strains of Bacillus thuringiensis.

FIG. 2A depicts the control of Spodoptera exigua development with Vip3Aa11 alone and in combination with zwittermicin A. FIG. 2B depicts the mortality of Spodoptera exigua with Vip3Aa11 alone and in combination with zwittermicin A.

FIG. 3A depicts the control of Spodoptera exigua development with Vip3Aa11 alone and in combination with zwittermicin A. FIG. 3B depicts the control of Trichoplusia ni development with Vip3Aa11 alone and in combination with zwittermicin A. FIG. 3C depicts the control of Plutella xylostella development with Vip3Aa11 alone and in combination with zwittermicin A. FIG. 3D depicts the control of Plutella xylostella development with Vip3Aa11 alone and in combination with zwittermicin A where the Plutella xylostella is resistant to treatment with DIPEL® (Bacillus thuringiensis subsp. kurstaki strain HD1).

FIG. 4A depicts the mortality of Spodoptera exigua with Cry1Ab1 alone and in combination with zwittermicin A. FIG. 4B depicts the mortality of Spodoptera exigua with Cry1Ia2 alone and in combination with zwittermicin A. FIG. 4C depicts the mortality of Spodoptera exigua with Cry2Ab1 alone and in combination with zwittermicin A.

FIG. 5 depicts the mortality of Spodoptera exigua treated with whole broths from several Bacillus thuringiensis strains.

FIG. 6A depicts the control of feeding by second instars of Spodoptera exigua treated with whole broths from several Bacillus thuringiensis strains. FIG. 6B depicts the control of feeding by third instars of Spodoptera exigua treated with whole broths from several Bacillus thuringiensis strains.

### DETAILED DESCRIPTION

The microorganisms and particular strains described herein, unless specifically noted otherwise, are all separated from nature and grown under artificial conditions such as in shake flask cultures or through scaled-up manufacturing processes, such as in bioreactors to maximize bioactive metabolite production, for example. Growth under such conditions leads to strain “domestication.” Generally, such a “domesticated” strain differs from its counterparts found in nature in that it is cultured as a homogenous population that is not subject to the selection pressures found in the natural environment but rather to artificial selection pressures.

As used herein, the verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”.

To “control” insects means to inhibit, through a toxic effect, the ability of insect pests to survive, grow, feed, or reproduce, or to limit insect-related damage or loss in crop plants or to protect the yield potential of a crop when grown in the presence of insect pests. To “control” insects may or may not mean killing the insects, although it preferably means killing the insects.

The compositions of the present invention comprise a biologically pure culture of a Bacillus thuringiensis strain or a cell-free preparation thereof comprising zwittermicin, and Vip3Aa, Cry1Aa, and/or Cry1Ab, wherein expression of zwittermicin A with Vip3Aa, Cry1Aa, and/or Cry1Ab results in a synergistic insecticidal effect. Such composition may further comprise Cry1Ca and/or Cry1Da, wherein expression of zwittermicin A with Vip3Aa, Cry1Aa, Cry1Ca, Cry1Da and/or Cry1Ab results in a synergistic insecticidal effect. Alternatively, the compositions may comprise a biologically pure culture of a Bacillus thuringiensis strain or a cell-free preparation thereof comprising zwittermicin, Vip3Aa, Cry1Aa, Cry1Ia2, Cry2Ab1 and/or Cry1Ab, wherein expression of zwittermicin A with Vip3Aa11, Cry1Ia2, Cry2Ab1, Cry1Aa11, and/or Cry1Ab1 results in a synergistic insecticidal effect.

In one aspect, the composition comprises Vip3Aa expressed from a gene comprising a DNA sequence exhibiting at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 1. In another aspect, the composition comprises Vip3Aa7 expressed from a gene comprising SEQ ID NO: 1. In another aspect the composition comprises Vip3Aa7, Vip3Aa10, Vip3Aa11, Vip3Aa12, and/or Vip3Aa15.

In one aspect, the composition comprises Cry1Ia2 expressed from a gene comprising a DNA sequence exhibiting at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 2. In another aspect, the composition comprises Cry1Ia2 expressed from a gene comprising SEQ ID NO: 2.

In one aspect, the composition comprises Cry2Ab expressed from a gene comprising a DNA sequence exhibiting at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 3.

In one aspect, the composition comprises Cry1Aa expressed from a gene comprising a DNA sequence exhibiting at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 4. In another aspect, the composition comprises Cry1Aa8 expressed from a gene comprising SEQ ID NO: 4. In another aspect, the composition comprises Cry1Aa expressed from a gene comprising a DNA sequence exhibiting at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 5. In another aspect, the composition comprises Cry1Aa3 expressed from a gene comprising SEQ ID NO: 5. In another aspect, the composition comprises Cry1Aa3, Cry1Aa8, Cry1Aa11 and/or Cry1Aa12.

In one aspect, the composition comprises Cry1Ab expressed from a gene comprising a DNA sequence exhibiting at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 6. In another aspect, the composition comprises Cry1Ab1 expressed from a gene comprising SEQ ID NO: 6. In another aspect, the composition comprises Cry1Ab1 expressed from a gene comprising a DNA sequence exhibiting at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 7. In another aspect, the composition comprises Cry1Ab1 expressed from a gene having 99.9% sequence identity to SEQ ID NO: 7. In another aspect, the composition comprises Cry1Ab1, Cry1Ab3, Cry1Ab4, Cry1Ab9, Cry1Ab12, Cry1Ab13, and/or Cry1Ab15.

In one aspect, the composition comprises Cry1Ca expressed from a gene comprising a DNA sequence exhibiting at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 16. In another aspect, the composition comprises Cry1Ca8 expressed from a gene comprising SEQ ID NO: 16. In another aspect, the composition comprises Cry1Ca1 or Cry1Ca8.

In one aspect, the composition comprises Cry1Da expressed from a gene comprising a DNA sequence exhibiting at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 18. In another aspect, the composition comprises Cry1Da1 expressed from a gene comprising SEQ ID NO: 18.

It is important to note that the proteins expressed by the genes set forth above may be expressed by different nucleic acid sequences yielding the same amino acid sequence. The following groups of proteins having the same prefix but different final numbers (e.g., Cry1Aa3 and Cry1Aa12 have the same amino acid sequence but are expressed from different nucleic acid sequences): Cry1Aa3 and Cry1Aa12; Cry1Ab1, Cry1Ab3, Cry1Ab4, Cry1Ab9, Cry1Ab12, Cry1Ab13, and Cry1Ab15; Cry1Ca8 and Cry1Ca9; Vip3Aa7, Vip3Aa10, Vip3Aa11, Vip3Aa12, and Vip3Aa15.

In one embodiment, the zwittermicin A in the composition is present in an amount at least 5-fold, at least 10-fold, at least 15-fold, at least 20-fold, at least 25-fold, at least 30-fold, at least 35-fold, at least 40-fold, at least 45-fold, or at least 50-fold greater than that in a biologically pure culture of Bacillus thuringiensis subsp. kurstaki strain EG7841.

In some embodiments, the zwittermicin A in the composition is present in an amount between 5-fold and 10-fold, between 5-fold and 20-fold, between 5-fold and 30-fold, between 5-fold and 40-fold, or between 5-fold and 50-fold that in a biologically pure culture of Bacillus thuringiensis subsp. kurstaki strain EG7841. In other embodiments, the zwittermicin A in the composition is present in an amount between 25-fold and 30-fold, between 25-fold and 35-fold, between 25-fold and 40-fold, between 25-fold and 45-fold, or between 25-fold and 50-fold that in a biologically pure culture of Bacillus thuringiensis subsp. kurstaki strain EG7841.

In one aspect, the zwittermicin A in the composition is present in an amount at least 2-fold, at least 3-fold, at least 4-fold, at least 5-fold, at least 6-fold, at least 7-fold, at least 8-fold, at least 9-fold, at least 10-fold, at least 11-fold, at least 12-fold, or at least 13-fold greater than that in a biologically pure culture of Bacillus thuringiensis subsp. aizawai strain ABTS-1857.

In some embodiments, the zwittermicin A in the composition is present in an amount between 2-fold and 4-fold, between 2-fold and 5-fold, between 2-fold and 6-fold, between 2-fold and 7-fold, between 2-fold and 8-fold, between 2-fold and 9-fold, between 2-fold and 10-fold, between 2-fold and 11-fold, between 2-fold and 12-fold, or between 2-fold and 13-fold that in a biologically pure culture of Bacillus thuringiensis subsp. aizawai strain ABTS-1857.

In one embodiment, an insecticidal mutant strain of the Bacillus thuringiensis strain NRRL B-67685, Bacillus thuringiensis strain NRRL B-67687, or Bacillus thuringiensis strain NRRL B-67688 is provided. The term “mutant” refers to a genetic variant derived from the Bacillus thuringiensis strain. In one embodiment, the mutant has one or more or all the identifying (functional) characteristics of Bacillus thuringiensis strain NRRL B-67685, Bacillus thuringiensis strain NRRL B-67687, or Bacillus thuringiensis strain NRRL B-67688. In a particular instance, the mutant or a fermentation product thereof controls (as an identifying functional characteristic) insects at least as well as the parent Bacillus thuringiensis strain NRRL B-67685, Bacillus thuringiensis strain NRRL B-67687, or Bacillus thuringiensis strain NRRL B-67688. Such mutants may be genetic variants having a genomic sequence that has greater than about 85%, greater than about 90%, greater than about 95%, greater than about 98%, or greater than about 99% sequence identity to Bacillus thuringiensis strain NRRL B-67685, Bacillus thuringiensis strain NRRL B-67687, or Bacillus thuringiensis strain NRRL B-67688. Mutants may be obtained by treating cells of Bacillus thuringiensis strain NRRL B-67685, Bacillus thuringiensis strain NRRL B-67687, or Bacillus thuringiensis strain NRRL B-67688 with chemicals or irradiation or by selecting spontaneous mutants from a population of Bacillus thuringiensis strain NRRL B-67685, Bacillus thuringiensis strain NRRL B-67687, or Bacillus thuringiensis strain NRRL B-67688 cells (such as phage resistant or antibiotic resistant mutants), by genome shuffling, as described below, or by other means well known to those practiced in the art.

Genome shuffling among Bacillus thuringiensis strains can be facilitated through the use of a process called protoplast fusion. The process begins with the formation of protoplasts from vegetative bacillary cells. The removal of peptidoglycan cell wall, typically using lysozyme and an osmotic stabilizer, results in the formation of a protoplast. This process is visible under a light microscope with the appearance of spherical cells. Addition of polyethylene glycol, PEG, then induces fusion among protoplasts, allowing genetic contents of two or more cells to come in contact facilitating recombination and genome shuffling. Fused cells then reparation and are recovered on a solid growth medium. During recovery, protoplasts rebuild peptidoglycan cell walls, transitioning back to bacillary shape. See Schaeffer, et al., (1976) PNAS USA, vol. 73, 6:2151-2155).

In some embodiments, the present invention provides a composition comprising a) zwittermicin A; and b) a Vip3A protein in a synergistically effective amount. In one aspect, the composition comprises a fermentation product of a bacterial strain expressing the zwittermicin A and Vip3A. In one embodiment, the bacterial strain is Escherichia coli. In another embodiment, the bacterial strain is a Bacillus sp. strain (e.g., Bacillus thuringiensis). In another aspect, the Vip3A is provided as a fermentation product of an E. coli strain that expresses Vip3A or a cell-free preparation of such E. coli strain. In such aspect, the zwittermycin may be provided separately as a purified compound, a fermentation product of a Bacillus thuringiensis strain that expresses zwittermicin, or as a purified or partially purified extract of such fermentation product.

In some embodiments, the synergistically effective amount refers to a synergistic weight ratio. In one aspect, the synergistic weight ratio of a) zwittermicin A; and b) a Vip3A protein lies in the range of 1:500 to 1000:1, preferably in the range of 1:500 to 500:1, more preferably in the range of 1:500 to 300:1. In other aspects, the synergistic weight ratio of a) zwittermicin A; and b) a Vip3A protein lies in the range of 1:1000 to 1000:1, 1:100 to 100:1, 1:50 to 50:1, 1:25 to 25:1, 1:10 to 10:1, 1:5 to 5:1, or 1:2 to 2:1.

In one embodiment, the Vip3A protein comprises an amino acid sequence exhibiting at least 75% sequence identity, at least 80% sequence identity, at least 90% sequence identity, at least 95% sequence identity, at least 96% sequence identity, at least 97% sequence identity, at least 98% sequence identity, or at least 99% sequence identity to SEQ ID NO: 8.

The present invention also encompasses methods of treating a plant to control animal pests by administering to a plant or a plant part, such as a leaf, stem, flowers, fruit, root, or seed or by applying to a locus on which plant or plant parts grow, such as soil, the disclosed Bacillus thuringiensis strains or mutants thereof, or cell-free preparations thereof or metabolites thereof.

In a method according to the invention a composition containing a disclosed Bacillus thuringiensis strain or an insecticidal mutant thereof can be applied to any plant or any part of any plant grown in any type of media used to grow plants (e.g., soil, vermiculite, shredded cardboard, and water) or applied to plants or the parts of plants grown aerially, such as orchids or staghorn ferns. The composition may for instance be applied by spraying, atomizing, vaporizing, scattering, dusting, watering, squirting, sprinkling, pouring or fumigating. As already indicated above, application may be carried out at any desired location where the plant of interest is positioned, such as agricultural, horticultural, forest, plantation, orchard, nursery, organically grown crops, turfgrass and urban environments.

Compositions of the present invention can be obtained by culturing the disclosed Bacillus thuringiensis strains or an insecticidal mutant (strain) derived therefrom according to methods well known in the art, including by using the media and other methods described in the examples below. Conventional large-scale microbial culture processes include submerged fermentation, solid state fermentation, or liquid surface culture. Towards the end of fermentation, as nutrients are depleted, cells begin the transition from growth phase to sporulation phase, such that the final product of fermentation is largely spores, metabolites and residual fermentation medium. Sporulation is part of the natural life cycle of Bacillus thuringiensis and is generally initiated by the cell in response to nutrient limitation. Fermentation is configured to obtain high levels of colony forming units of and to promote sporulation. The bacterial cells, spores and metabolites in culture media resulting from fermentation may be used directly or concentrated by conventional industrial methods, such as centrifugation, tangential-flow filtration, depth filtration, and evaporation.

Compositions of the present invention include fermentation products. In some embodiments, the concentrated fermentation broth is washed, for example, via a diafiltration process, to remove residual fermentation broth and metabolites. The term “broth concentrate,” as used herein, refers to whole broth (fermentation broth) that has been concentrated by conventional industrial methods, as described above, but remains in liquid form. The term “fermentation solid,” as used herein, refers to the solid material that remains after the fermentation broth is dried. The term “fermentation product,” as used herein, refers to whole broth, broth concentrate and/or fermentation solids. Compositions of the present invention include fermentation products.

The fermentation broth or broth concentrate can be dried with or without the addition of carriers using conventional drying processes or methods such as spray drying, freeze drying, tray drying, fluidized-bed drying, drum drying, or evaporation.

The resulting dry products may be further processed, such as by milling or granulation, to achieve a specific particle size or physical format. Carriers, described below, may also be added post-drying.

Cell-free preparations of fermentation broth of the strains of the present invention can be obtained by any means known in the art, such as extraction, centrifugation and/or filtration of fermentation broth. Those of skill in the art will appreciate that so-called cell-free preparations may not be devoid of cells but rather are largely cell-free or essentially cell-free, depending on the technique used (e.g., speed of centrifugation) to remove the cells. The resulting cell-free preparation may be dried and/or formulated with components that aid in its application to plants or to plant growth media. Concentration methods and drying techniques described above for fermentation broth are also applicable to cell-free preparations.

In certain aspects, the fermentation product further comprises a formulation ingredient. The formulation ingredient may be a wetting agent, extender, solvent, spontaneity promoter, emulsifier, dispersant, frost protectant, thickener, and/or an adjuvant. In one embodiment, the formulation ingredient is a wetting agent. In other aspects, the fermentation product is a freeze-dried powder or a spray-dried powder.

Compositions of the present invention may include formulation ingredients added to compositions of the present invention to improve recovery, efficacy, or physical properties and/or to aid in processing, packaging and administration. Such formulation ingredients may be added individually or in combination.

The formulation ingredients may be added to compositions comprising cells, cell-free preparations, isolated compounds, and/or metabolites to improve efficacy, stability, and physical properties, usability and/or to facilitate processing, packaging and end-use application. Such formulation ingredients may include agriculturally acceptable carriers, inerts, stabilization agents, preservatives, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the formulation ingredient is a binder, adjuvant, or adhesive that facilitates adherence of the composition to a plant part, such as leaves, seeds, or roots. See, for example, Taylor, A. G., et al., “Concepts and Technologies of Selected Seed Treatments,” Annu. Rev. Phytopathol., 28: 321-339 (1990). The stabilization agents may include anti-caking agents, anti-oxidation agents, anti-settling agents, antifoaming agents, desiccants, protectants or preservatives. The nutrients may include carbon, nitrogen, and phosphorus sources such as sugars, polysaccharides, oil, proteins, amino acids, fatty acids and phosphates. The physical property modifiers may include bulking agents, wetting agents, thickeners, pH modifiers, rheology modifiers, dispersants, adjuvants, surfactants, film-formers, hydrotropes, builders, antifreeze agents or colorants. In some embodiments, the composition comprising cells, cell-free preparation and/or metabolites produced by fermentation can be used directly with or without water as the diluent without any other formulation preparation. In a particular embodiment, a wetting agent, or a dispersant, is added to a fermentation solid, such as a freeze-dried or spray-dried powder. In some embodiments, the formulation inerts are added after concentrating fermentation broth and/or during and/or after drying. A wetting agent increases the spreading and penetrating properties, or a dispersant increases the dispersability and solubility of the active ingredient (once diluted) when it is applied to surfaces. Exemplary wetting agents are known to those of skill in the art and include sulfosuccinates and derivatives, such as MULTIWET™ MO-70R (Croda Inc., Edison, N.J.); siloxanes such as BREAK-THRU® (Evonik, Germany); nonionic compounds, such as ATLOX™ 4894 (Croda Inc., Edison, N.J.); alkyl polyglucosides, such as TERWET® 3001 (Huntsman International LLC, The Woodlands, Tex.); C12-C14 alcohol ethoxylate, such as TERGITOL® 15-S-15 (The Dow Chemical Company, Midland, Mich.); phosphate esters, such as RHODAFAC® BG-510 (Rhodia, Inc.); and alkyl ether carboxylates, such as EMULSOGEN™ LS (Clariant Corporation, North Carolina).

In one embodiment, the fermentation product comprises at least about 1×10⁴ colony forming units (CFU) of the microorganism (e.g., Bacillus thuringiensis strain NRRL B-67685, Bacillus thuringiensis strain NRRL B-67687, Bacillus thuringiensis strain NRRL B-67688, or an insecticidal mutant strain thereof)/mL broth. In another embodiment, the fermentation product comprises at least about 1×10⁵ colony forming units (CFU) of the microorganism/mL broth. In another embodiment, the fermentation product comprises at least about 1×10⁶ CFU of the microorganism/mL broth. In yet another embodiment, the fermentation product comprises at least about 1×10⁷ CFU of the microorganism/mL broth. In another embodiment, the fermentation product comprises at least about 1×10⁸ CFU of the microorganism/mL broth. In another embodiment, the fermentation product comprises at least about 1×10⁹ CFU of the microorganism/mL broth. In another embodiment, the fermentation product comprises at least about 1×10¹⁹ CFU of the microorganism/mL broth. In another embodiment, the fermentation product comprises at least about 1×10¹¹ CFU of the microorganism/mL broth.

The inventive compositions can be used as such or, depending on their particular physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, gas (under pressure), gas generating product, foams, pastes, pesticide coated seed, suspension concentrates, oil dispersion, suspo-emulsion concentrates, soluble concentrates, suspensions, wettable powders, soluble powders, dusts and granules, water-soluble and water-dispersible granules or tablets, water-soluble and water-dispersible powders for the treatment of seed, wettable powders, natural products and synthetic substances impregnated with active ingredient, and also microencapsulations in polymeric substances and in coating materials for seed, and also ULV cold-fogging and warm-fogging formulations.

In some embodiments, the inventive compositions are liquid formulations. Non-limiting examples of liquid formulations include suspension concentrations and oil dispersions. In other embodiments, the inventive compositions are solid formulations. Non-limiting examples of solid formulations include freeze-dried powders and spray-dried powders.

All plants and plant parts can be treated in accordance with the invention. In the present context, plants are understood as meaning all plants and plant populations, such as desired and undesired wild plants or crop plants (including naturally occurring crop plants). Crop plants can be plants which can be obtained by traditional breeding and optimization methods or by biotechnological and recombinant methods, or combinations of these methods, including the transgenic plants and including the plant varieties capable or not of being protected by Plant Breeders' Rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include crop material and vegetative and generative propagation material, for example cuttings, tubers, rhizomes, slips and seeds.

As has already been mentioned above, all plants and their parts may be treated in accordance with the invention. In a preferred embodiment, plant species and plant varieties, and their parts, which grow wild or which are obtained by traditional biological breeding methods such as hybridization or protoplast fusion are treated. In a further preferred embodiment, transgenic plants and plant varieties which have been obtained by recombinant methods, if appropriate in combination with traditional methods (genetically modified organisms), and their parts are treated. The term “parts” or “parts of plants” or “plant parts” has been explained hereinabove. Plants of the plant varieties which are in each case commercially available or in use are especially preferably treated in accordance with the invention. Plant varieties are understood as meaning plants with novel traits which have been bred both by traditional breeding, by mutagenesis or by recombinant DNA techniques. They may take the form of varieties, races, biotypes and genotypes.

The treatment of the plants and plant parts with the compositions according to the invention is carried out directly or by acting on the environment, habitat or storage space using customary treatment methods, for example by dipping, spraying, atomizing, misting, evaporating, dusting, fogging, scattering, foaming, painting on, spreading, injecting, drenching, trickle irrigation and, in the case of propagation material, in particular in the case of seed, furthermore by the dry seed treatment method, the wet seed treatment method, the slurry treatment method, by encrusting, by coating with one or more coats and the like. It is furthermore possible to apply the active substances by the ultra-low volume method or to inject the active substance preparation or the active substance itself into the soil.

A preferred direct treatment of the plants is the leaf application treatment, i.e., compositions according to the invention are applied to the foliage, it being possible for the treatment frequency and the application rate to be matched to the infection pressure of the pathogen in question.

In the case of systemically active agents, the compositions according to the invention reach the plants via the root system. In this case, the treatment of the plants is effected by allowing the compositions according to the invention to act on the environment of the plant. This can be done for example by drenching, incorporating in the soil or into the nutrient solution, i.e., the location of the plant (for example the soil or hydroponic systems) is impregnated with a liquid form of the compositions according to the invention, or by soil application, i.e., the compositions according to the invention are incorporated into the location of the plants in solid form (for example in the form of granules). In the case of paddy rice cultures, this may also be done by metering the compositions according to the invention into a flooded paddy field in a solid use form (for example in the form of granules).

Preferred plants are those from the group of the useful plants, ornamentals, turfs, generally used trees which are employed as ornamentals in the public and domestic sectors, and forestry trees. Forestry trees comprise trees for the production of timber, cellulose, paper and products made from parts of the trees.

The term “useful plants” as used in the present context refers to crop plants which are employed as plants for obtaining foodstuffs, feedstuffs, fuels or for industrial purposes.

The useful plants which can be treated and/or improved with the compositions and methods of the present invention include for example the following types of plants: turf, vines, cereals, for example wheat, barley, rye, oats, rice, maize and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cacao and peanuts; cucurbits, for example pumpkin/squash, cucumbers and melons; fibre plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or else plants such as tobacco, nuts, coffee, aubergine, sugar cane, tea, pepper, grapevines, hops, bananas, latex plants and ornamentals, for example flowers, shrubs, deciduous trees and coniferous trees. This enumeration is no limitation.

The following plants are considered to be particularly suitable target crops for applying compositions and methods of the present invention: cotton, aubergine, turf, pome fruit, stone fruit, soft fruit, maize, wheat, barley, cucumber, tobacco, vines, rice, cereals, pear, beans, soybeans, oilseed rape, tomato, bell pepper, melons, cabbage, potato and apple.

Additional useful plants include cereals, for example wheat, rye, barley, triticale, oats or rice; beet, for example sugar beet or fodder beet; fruits, such as pomes, stone fruits or soft fruits, for example apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as broccoli, spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; natural rubber plants or ornamental and forestry plants, such as flowers, shrubs, broad-leaved trees or evergreens, for example conifers; and on the plant propagation material, such as seeds, and the crop material of these plants.

In a preferred embodiment of the invention, the useful plant is selected from soybean, corn, wheat, triticale, barley, oat, rye, rape, millet, rice, sunflower, cotton, sugar beet, pome fruit, stone fruit, citrus, banana, strawberry, blueberry, almond, grape, mango, papaya, peanut, potato, tomato, pepper, cucurbit, cucumber, melon, watermelon, garlic, onion, broccoli, carrot, cabbage, bean, dry bean, canola, pea, lentil, alfalfa, trefoil, clover, flax, elephant grass, grass, lettuce, sugarcane, tea, tobacco and coffee; each in its natural or genetically modified form.

The Bacillus thuringiensis strains according to the invention, in combination with good plant tolerance and favorable toxicity to warm-blooded animals and being tolerated well by the environment, are suitable for protecting plants and plant organs, for increasing harvest yields, for improving the quality of the harvested material and for controlling animal pests, in particular insects, which are encountered in agriculture, in horticulture, in animal husbandry, in forests, in gardens and leisure facilities, in protection of stored products and of materials, and in the hygiene sector. They can be preferably employed as plant protection agents. They are active against normally sensitive and resistant species and against all or some stages of development. The abovementioned pests include:

pests from the phylum Arthropoda, especially from the class Arachnida, for example Acarus spp., Aceria kuko, Aceria sheldoni, Aculops spp., Aculus spp., Amblyomma spp., Amphitetranychus viennensis, Argas spp., Boophilus spp., Brevipalpus spp., Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., Epitrimerus pyri, Eutetranychus spp., Eriophyes spp., Glycyphagus domesticus, Halotydeus destructor, Hemitarsonemus spp., Hyalomma spp., Ixodes spp., Latrodectus spp., Loxosceles spp., Metatetranychus spp., Neutrombicula autumnalis, Nuphersa spp., Oligonychus spp., Ornithodorus spp., Ornithonyssus spp., Panonychus spp., Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., Tetranychus spp., Trombicula alfreddugesi, Vaejovis spp., Vasates lycopersici;

from the class Chilopoda, for example, Geophilus spp., Scutigera spp.;

from the order or the class Collembola, for example, Onychiurus armatus, Sminthurus viridis;

from the class Diplopoda, for example, Blaniulus guttulatus;

from the class Insecta, e.g., from the order Blattodea, for example Blatta orientalis, Blattella asahinai, Blattella germanica, Leucophaea maderae, Loboptera decipiens, Neostylopyga rhombifolia, Panchlora spp., Parcoblatta spp., Periplaneta spp., Pycnoscelus surinamensis, Supella longipalpa;

from the order Coleoptera, for example, Acalymma vittatum, Acanthoscelides obtectus, Adoretus spp., Aethina tumida, Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Baris caerulescens, Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptolestes ferrugineus, Cryptorhynchus lapathi, Cylindrocopturus spp., Dermestes spp., Diabrotica spp., Dichocrocis spp., Dicladispa armigera, Diloboderus spp., Epicaerus spp., Epilachna spp., Epitrix spp., Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., Lachnosterna consanguinea, Lasioderma serricorne, Latheticus oryzae, Lathridius spp., Lema spp., Leptinotarsa decemlineata, Leucoptera spp., Lissorhoptrus oryzophilus, Listronotus (=Hyperodes) spp., Lixus spp., Luperodes spp., Luperomorpha xanthodera, Lyctus spp., Megascelis spp., Melanotus spp., Meligethes aeneus, Melolontha spp., Migdolus spp., Monochamus spp., Naupactus xanthographus, Necrobia spp., Neogalerucella spp., Niptus hololeucus, Oryctes rhinoceros, Oryzaephilus surinamensis, Oryzaphagus oryzae, Otiorrhynchus spp., Oulema spp., Oulema melanopus, Oulema oryzae, Oxycetonia jucunda, Phaedon cochleariae, Phyllophaga spp., Phyllophaga helleri, Phyllotreta spp., Popillia japonica, Premnotrypes spp., Prostephanus truncatus, Psylliodes spp., Ptinus spp., Rhizobius ventralis, Rhizopertha dominica, Rhynchophorus spp., Rhynchophorus ferrugineus, Rhynchophorus palmarum, Sinoxylon perforans, Sitophilus spp., Sitophilus oryzae, Sphenophorus spp., Stegobium paniceum, Sternechus spp., Symphyletes spp., Tanymecus spp., Tenebrio molitor, Tenebrioides mauretanicus, Tribolium spp., Trogoderma spp., Tychius spp., Xylotrechus spp., Zabrus spp.;

from the order Dermaptera, for example Anisolabis maritime, Forficula auricularia, Labidura riparia;

from the order Diptera, for example Aedes spp., Agromyza spp., Anastrepha spp., Anopheles spp., Asphondylia spp., Bactrocera spp., Bibio hortulanus, Calliphora erythrocephala, Calliphora vicina, Ceratitis capitata, Chironomus spp., Chrysomya spp., Chrysops spp., Chrysozona pluvialis, Cochliomyia spp., Contarinia spp., Cordylobia anthropophaga, Cricotopus sylvestris, Culex spp., Culicoides spp., Culiseta spp., Cuterebra spp., Dacus oleae, Dasineura spp., Delia spp., Dermatobia hominis, Drosophila spp., Echinocnemus spp., Euleia heraclei, Fannia spp., Gasterophilus spp., Glossina spp., Haematopota spp., Hydrellia spp., Hydrellia griseola, Hylemya spp., Hippobosca spp., Hypoderma spp., Liriomyza spp., Lucilia spp., Lutzomyia spp., Mansonia spp., Musca spp., Oestrus spp., Oscinella frit, Paratanytarsus spp., Paralauterborniella subcincta, Pegomyia or Pegomya spp., Phlebotomus spp., Phorbia spp., Phormia spp., Piophila casei, Platyparea poeciloptera, Prodiplosis spp., Psila rosae, Rhagoletis spp., Sarcophaga spp., Simulium spp., Stomoxys spp., Tabanus spp., Tetanops spp., Tipula spp., Toxotrypana curvicauda;

from the order Hemiptera, for example, Acizzia acaciaebaileyanae, Acizzia dodonaeae, Acizzia uncatoides, Acrida turrita, Acyrthosiphon spp., Acrogonia spp., Aeneolamia spp., Agonoscena spp., Aleurocanthus spp., Aleyrodes proletella, Aleurolobus barodensis, Aleurothrixus floccosus, Allocaridara malayensis, Amrasca spp., Anuraphis cardui, Aonidiella spp., Aphanostigma pini, Aphis spp., Arboridia apicalis, Arytainilla spp., Aspidiella spp., Aspidiotus spp., Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., Calligypona marginata, Capulinia spp., Cameocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus aonidum, Chrysomphalus ficus, Cicadulina mbila, Coccomytilus halli, Coccus spp., Cryptomyzus ribis, Cryptoneossa spp., Ctenarytaina spp., Dalbulus spp., Dialeurodes chittendeni, Dialeurodes citri, Diaphorina citri, Diaspis spp., Diuraphis spp., Doralis spp., Drosicha spp., Dysaphis spp., Dysmicoccus spp., Empoasca spp., Eriosoma spp., Erythroneura spp., Eucalyptolyma spp., Euphyllura spp., Euscelis bilobatus, Ferrisia spp., Fiorinia spp., Furcaspis oceanica, Geococcus coffeae, Glycaspis spp., Heteropsylla cubana, Heteropsylla spinulosa, Homalodisca coagulata, Hyalopterus arundinis, Hyalopterus pruni, Icerya spp., Idiocerus spp., Idioscopus spp., Laodelphax striatellus, Lecanium spp., Lepidosaphes spp., Lipaphis erysimi, Lopholeucaspis japonica, Lycorma delicatula, Macrosiphum spp., Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., Nasonovia ribisnigri, Neomaskellia spp., Nephotettix spp., Nettigoniclla spectra, Nilaparvata lugens, Oncometopia spp., Orthezia praelonga, Oxya chinensis, Pachypsylla spp., Parabemisia myricae, Paratrioza spp., Parlatoria spp., Pemphigus spp., Peregrinus maidis, Perkinsiella spp., Phenacoccus spp., Phloeomyzus passerinii, Phorodon humuli, Phylloxera spp., Pinnaspis aspidistrae, Planococcus spp., Prosopidopsylla flava, Protopulvinaria pyriformis, Pseudaulacaspis pentagona, Pseudococcus spp., Psyllopsis spp., Psylla spp., Pteromalus spp., Pulvinaria spp., Pyrilla spp., Quadraspidiotus spp., Quesada gigas, Rastrococcus spp., Rhopalosiphum spp., Saissetia spp., Scaphoideus titanus, Schizaphis graminum, Selenaspidus articulatus, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., Trialeurodes vaporariorum, Trioza spp., Typhlocyba spp., Unaspis spp., Viteus vitifolii, Zygina spp.;

from the suborder Heteroptera, for example, Aelia spp., Anasa tristis, Antestiopsis spp., Boisea spp., Blissus spp., Calocoris spp., Campylomma livida, Cavelerius spp., Cimex spp., Collaria spp., Creontiades dilutus, Dasynus piperis, Dichelops furcatus, Diconocoris hewetti, Dysdercus spp., Euschistus spp., Eurydema spp., Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., Lygus spp., Macropes excavatus, Megacopta cribraria, Miridae, Monalonion atratum, Nezara spp., Nysius spp., Oebalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., Triatoma spp.;

from the order Hymenoptera, for example, Acromyrmex spp., Athalia spp., Atta spp., Camponotus spp., Dolichovespula spp., Diprion spp., Hoplocampa spp., Lasius spp., Linepithema (Iridiomyrmex) humile, Monomorium pharaonic, Paratrechina spp., Paravespula spp., Plagiolepis spp., Sirex spp., Solenopsis invicta, Tapinoma spp., Technomyrmex albipes, Urocerus spp., Vespa spp., Wasmannia auropunctata, Xeris spp.;

from the order Isopoda, for example, Armadillidium vulgare, Oniscus asellus, Porcellio scaber;

from the order Isoptera, for example, Coptotermes spp., Cornitermes cumulans, Cryptotermes spp., Incisitermes spp., Kalotermes spp., Microtermes obesi, Nasutitermes spp., Odontotermes spp., Porotermes spp., Reticulitermes spp.;

from the order Lepidoptera, for example, Achroia grisella, Acronicta major, Adoxophyes spp., Aedia leucomelas, Agrotis spp., Alabama spp., Amyelois transitella, Anarsia spp., Anticarsia spp., Argyroploce spp., Autographa spp., Barathra brassicae, Blastodacna atra, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., Choreutis pariana, Choristoneura spp., Chrysodeixis chalcites, Clysia ambiguella, Cnaphalocerus spp., Cnaphalocrocis medinalis, Cnephasia spp., Conopomorpha spp., Conotrachelus spp., Copitarsia spp., Cydia spp., Dalaca noctuides, Diaphania spp., Diparopsis spp., Diatraea saccharalis, Earias spp., Ecdytolopha aurantium, Elasmopalpus lignosellus, Eldana saccharina, Ephestia spp., Epinotia spp., Epiphyas postvittana, Erannis spp., Erschoviella musculana, Etiella spp., Eudocima spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Lampides spp., Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Omphisa spp., Operophtera spp., Oria spp., Orthaga spp., Ostrinia spp., Panolis flammea, Parnara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella (=Plutella maculipennis), Prays spp., Prodenia spp., Protoparce spp., Pseudaletia spp., Pseudaletia unipuncta, Pseudoplusia includens, Pyrausta nubilalis, Rachiplusia nu, Schoenobius spp., Scirpophaga spp., Scirpophaga innotata, Scotia segetum, Sesamia spp., Sesamia inferens, Sparganothis spp., Spodoptera spp., Spodoptera praefica, Stathmopoda spp., Stenoma spp., Stomopteryx subsecivella, Synanthedon spp., Tecia solanivora, Thaumetopoea spp., Thermobia gemmatalis, Tinea cloacella, Tinea pellionella, Tineola bisselliella, Tortrix spp., Trichophaga tapetzella, Trichoplusia spp., Tryporyza incertulas, Tuta absoluta, Virachola spp.;

from the order Orthoptera or Saltatoria, for example, Acheta domesticus, Dichroplus spp., Gryllotalpa spp., Hieroglyphus spp., Locusta spp., Melanoplus spp., Paratlanticus ussuriensis, Schistocerca gregaria;

from the order Phthiraptera, for example Damalinia spp., Haematopinus spp., Linognathus spp., Pediculus spp., Phylloxera vastatrix, Phthirus pubis, Trichodectes spp.;

from the order Psocoptera, for example Lepinotus spp., Liposcelis spp.;

from the order Siphonaptera, for example Ceratophyllus spp., Ctenocephalides spp., Pulex irritans, Tunga penetrans, Xenopsylla cheopis;

from the order Thysanoptera, for example Anaphothrips obscurus, Baliothrips biformis, Chaetanaphothrips leeuweni, Drepanothrips reuteri, Enneothrips flavens, Frankliniella spp., Haplothrips spp., Heliothrips spp., Hercinothrips femoralis, Kakothrips spp., Rhipiphorothrips cruentatus, Scirtothrips spp., Taeniothrips cardamomi, Thrips spp.;

from the order Zygentoma (=Thysanura), for example Ctenolepisma spp., Lepisma saccharina, Lepismodes inquilinus, Thermobia domestica;

from the class Symphyla, for example, Scutigerella spp.;

pests from the phylum Mollusca, especially from the class Bivalvia, for example, Dreissena spp.,

and from the class Gastropoda, for example, Anion spp., Biomphalaria spp., Bulinus spp., Deroceras spp., Galba spp., Lymnaea spp., Oncomelania spp., Pomacea spp., Succinea spp.

The fact that the compositions are well tolerated by plants at the concentrations required for controlling plant diseases and pests allows the treatment of above-ground parts of plants, of propagation stock and seeds, and of the soil.

According to the invention all plants and plant parts can be treated including cultivars and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random and directed mutagenesis, molecular or genetic markers or by bioengineering and genetic engineering methods.

The inventive compositions, when they are well tolerated by plants, have favorable homeotherm toxicity and are well tolerated by the environment, are suitable for protecting plants and plant organs, for enhancing harvest yields, for improving the quality of the harvested material. They can preferably be used as crop protection compositions. They are active against normally sensitive and resistant species and against all or some stages of development.

Plants which can be treated in accordance with the invention include the following main crop plants: maize, soya bean, alfalfa, cotton, sunflower, Brassica oil seeds such as Brassica napus (e.g., canola, rapeseed), Brassica rapa, B. juncea (e.g., (field) mustard) and Brassica carinata, Arecaceae sp. (e.g., oilpalm, coconut), rice, wheat, sugar beet, sugar cane, oats, rye, barley, millet and sorghum, triticale, flax, nuts, grapes and vine and various fruit and vegetables from various botanic taxa, e.g., Rosaceae sp. (e.g., pome fruits such as apples and pears, but also stone fruits such as apricots, cherries, almonds, plums and peaches, and berry fruits such as strawberries, raspberries, red and black currant and gooseberry), Ribesioidae sp., Juglandaceae sp., Betulaceae sp., Anacardiaceae sp., Fagaceae sp., Moraceae sp., Oleaceae sp. (e.g., olive tree), Actinidaceae sp., Lauraceae sp. (e.g., avocado, cinnamon, camphor), Musaceae sp. (e.g., banana trees and plantations), Rubiaceae sp. (e.g., coffee), Theaceae sp. (e.g., tea), Sterculiceae sp., Rutaceae sp. (e.g., lemons, oranges, mandarins and grapefruit); Solanaceae sp. (e.g., tomatoes, potatoes, peppers, capsicum, aubergines, tobacco), Liliaceae sp., Compositae sp. (e.g., lettuce, artichokes and chicory—including root chicory, endive or common chicory), Umbelliferae sp. (e.g., carrots, parsley, celery and celeriac), Cucurbitaceae sp. (e.g., cucumbers—including gherkins, pumpkins, watermelons, calabashes and melons), Alliaceae sp. (e.g., leeks and onions), Cruciferae sp. (e.g., white cabbage, red cabbage, broccoli, cauliflower, Brussels sprouts, pak choi, kohlrabi, radishes, horseradish, cress and chinese cabbage), Leguminosae sp. (e.g., peanuts, peas, lentils and beans—e.g., common beans and broad beans), Chenopodiaceae sp. (e.g., Swiss chard, fodder beet, spinach, beetroot), Linaceae sp. (e.g., hemp), Cannabeacea sp. (e.g., cannabis), Malvaceae sp. (e.g., okra, cocoa), Papaveraceae (e.g., poppy), Asparagaceae (e.g., asparagus); useful plants and ornamental plants in the garden and woods including turf, lawn, grass and Stevia rebaudiana; and in each case genetically modified types of these plants.

Examples of trees which can be improved in accordance with the method according to the invention are: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved in accordance with the method according to the invention are: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiata, P. ponderosa, P. contorta, P. sylvestre, P. elliottii, P. montecola, P. albicaulis, P. resinosa, P. palustris, P. taeda, P. flexilis, P. jeffregi, P. baksiana, P. strobus; from the tree species Eucalyptus: E. grandis, E. globulus, E. camadentis, E. nitens, E. obliqua, E. regnans, E. pilularus.

Especially preferred trees which can be improved in accordance with the method according to the invention are: from the tree species Pinus: P. radiata, P. ponderosa, P. contorta, P. sylvestre, P. strobus; from the tree species Eucalyptus: E. grandis, E. globulus, E. camadentis.

Very particularly preferred trees which can be improved in accordance with the method according to the invention are: horse chestnut, Platanaceae, linden tree, maple tree.

The present invention can also be applied to any turf grasses, including cool-season turf grasses and warm-season turf grasses. Examples of cold-season turf grasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German mixed bentgrass (Agrostis spp. including Agrostis tenuis Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);

ryegrasses (Lolium spp.), such as annual ryegrass (Lolium multiflorum Lam.), perennial ryegrass (Lolium perenne L.) and Italian ryegrass (Lolium multiflorum Lam.);

and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.)

Examples of further cool-season turf grasses are beachgrass (Ammophila breviligulata Fern.), smooth bromegrass (Bromus inermis Leyss.), cattails such as timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.)

Examples of warm-season turf grasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpetgrass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), blue grama (Bouteloua gracilis (H.B.K.) Lag. ex Griffiths), seashore paspalum (Paspalum vaginatum Swartz) and sideoats grama (Bouteloua curtipendula (Michx. Torr.). Cool-season turf grasses are generally preferred for the use according to the invention. Especially preferred are bluegrass, benchgrass and redtop, fescues and ryegrasses. Bentgrass is especially preferred.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e., said plants have a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients or shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore by affected by improved plant architecture (under stress and non-stress conditions), including early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stress factors. Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g., in corn) be produced by detasseling, (i.e., the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in the hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e., plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e., plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxido-reductase enzyme. Glyphosate-tolerant plants can also be obtained by expressing a gene that encodes a glyphosate acetyl transferase enzyme. Glyphosate-tolerant plants can also be obtained by selecting plants containing naturally-occurring mutations of the above-mentioned genes.

Other herbicide resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase.

Further herbicide-tolerant plants are also plants that are made tolerant to the herbicides inhibiting the enzyme hydroxyphenylpyruvatedioxygenase (HPPD). Hydroxyphenylpyruvatedioxygenases are enzymes that catalyze the reaction in which parahydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme. Tolerance to HPPD-inhibitors can also be obtained by transforming plants with genes encoding certain enzymes enabling the formation of homogentisate despite the inhibition of the native HPPD enzyme by the HPPD-inhibitor. Tolerance of plants to HPPD inhibitors can also be improved by transforming plants with a gene encoding an enzyme prephenate dehydrogenase in addition to a gene encoding an HPPD-tolerant enzyme.

Still further herbicide resistant plants are plants that are made tolerant to acetolactate synthase (ALS) inhibitors. Known ALS-inhibitors include, for example, sulfonylurea, imidazolinone, triazolopyrimidines, pyrimidinyloxy(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides. The production of sulfonylurea-tolerant plants and imidazolinone-tolerant plants has been described. Other imidazolinone-tolerant plants have also been described. Further sulfonylurea- and imidazolinone-tolerant plants have also been described.

Other plants tolerant to imidazolinone and/or sulfonylurea can be obtained by induced mutagenesis, selection in cell cultures in the presence of the herbicide or mutation breeding as described for example for soya beans, for rice, for sugar beet, for lettuce or for sunflower.

Plants or plant cultivars (obtained by plant biotechnology methods such as genetic engineering) which may also be treated according to the invention are insect-resistant transgenic plants, i.e., plants made resistant to attack by certain target insects. Such plants can be obtained by genetic transformation, or by selection of plants containing a mutation imparting such insect resistance.

An “insect-resistant transgenic plant”, as used herein, includes any plant containing at least one transgene comprising a coding sequence encoding:
1) an insecticidal crystal protein from Bacillus thuringiensis or an insecticidal portion thereof, such as the insecticidal crystal proteins listed by Crickmore et al., Microbiology and Molecular Biology Reviews (1998), 62, 807-813, updated by Crickmore et al., (2005) in the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1F, Cry2Ab, Cry3Ae, or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, e.g., the Cry1A.105 protein produced by corn event MON98034; or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced into the encoding DNA during cloning or transformation, such as the Cry3Bb1 protein in corn events MON863 or MON88017, or the Cry3A protein in corn event MIR604; or
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1a and VIP2A proteins; or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes induced into the encoding DNA during cloning or transformation (while still encoding an insecticidal protein), such as the VIP3Aa protein in cotton event COT102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
a. plants which contain a transgene capable of reducing the expression and/or the activity of poly(ADP-ribose)polymerase (PARP) gene in the plant cells or plants.
b. plants which contain a stress tolerance enhancing transgene capable of reducing the expression and/or the activity of the PARG encoding genes of the plants or plants cells.
c. plants which contain a stress tolerance enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotine amide phosphoribosyltransferase.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesized starch in wild type plant cells or plants, so that this modified starch is better suited for special applications. Said transgenic plants synthesizing a modified starch have been described.
2) transgenic plants which synthesize non-starch carbohydrate polymers or which synthesize non starch carbohydrate polymers with altered properties in comparison to wild type plants without genetic modification. Examples are plants which produce polyfructose, especially of the inulin and levan-type, plants which produce alpha-1,4-glucans, plants which produce alpha-1,6 branched alpha-1,4-glucans, and plants producing alternan.
3) transgenic plants which produce hyaluronan.

Plants or plant cultivars (that can be obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants which contain an altered form of cellulose synthase genes;
b) plants, such as cotton plants which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, e.g., through downregulation of fibre-selective β-1,3-glucanase;
f) plants, such as cotton plants, which have fibres with altered reactivity, e.g., through the expression of N-acetylglucosaminetransferase gene including nodC and chitin synthase genes.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plant such as oilseed rape plants, which produce oil having a low level of saturated fatty acids.

Particularly useful transgenic plants which may be treated according to the invention are plants which comprise one or more genes which encode one or more toxins, are the following which are sold under the trade names YIELD GARD® (for example maize, cotton, soya beans), KNOCKOUT® (for example maize), BITEGARD® (for example maize), BT-XTRA® (for example maize), STARLINK® (for example maize), BOLLGARD® (cotton), NUCOTN® (cotton), NUCOTN 33B® (cotton), NATUREGARD® (for example maize), PROTECTA® and NEWLEAF® (potato). Examples of herbicide-tolerant plants which may be mentioned are maize varieties, cotton varieties and soya bean varieties which are sold under the trade names ROUNDUP READY® (tolerance to glyphosate, for example maize, cotton, soya beans), LIBERTY LINK® (tolerance to phosphinothricin, for example oilseed rape), IMI® (tolerance to imidazolinone) and SCS® (tolerance to sulphonylurea, for example maize). Herbicide-resistant plants (plants bred in a conventional manner for herbicide tolerance) which may be mentioned include the varieties sold under the name CLEARFIELD® (for example maize).

Particularly useful transgenic plants which may be treated according to the invention are plants containing transformation events, or a combination of transformation events, that are listed for example in the databases for various national or regional regulatory agencies.

The compositions according to the invention are particularly suitable for the treatment of seed. The combinations according to the invention which have been mentioned above as being preferred or especially preferred must be mentioned by preference in this context. Thus, a large proportion of the damage to crop plants which is caused by pests is already generated by infestation of the seed while the seed is stored and after the seed is introduced into the soil, and during and immediately after germination of the plants. This phase is particularly critical since the roots and shoots of the growing plant are particularly sensitive and even a small amount of damage can lead to the death of the whole plant. There is therefore in particular a great interest in protecting the seed and the germinating plant by using suitable compositions.

The control of pests by treating the seed of plants has been known for a long time and is the subject of continuous improvements. However, the treatment of seed poses a series of problems which cannot always be solved in a satisfactory manner Thus, it is desirable to develop methods of protecting the seed and the germinating plant which dispense with the additional application of plant protection compositions after sowing or after the emergence of the plants. It is furthermore desirable to optimize the amount of the compositions employed in such a way as to provide the best possible protection for the seed and the germinating plant against attack by pests. In particular, methods for the treatment of seed should also include the intrinsic fungicidal and/or insecticidal properties of transgenic plants in order to achieve an optimal protection of the seed and of the germinating plant while keeping the application rate of plant protection compositions as low as possible.

The present invention therefore particularly also relates to a method of protecting seed and germinating plants from attack by pests by treating the seed with a composition according to the invention.

In certain aspects, the compositions of the present invention are applied at about 1×10⁴ to about 1×10¹⁴ colony forming units (CFU) per hectare, at about 1×10⁴ to about 1×10¹² colony forming units (CFU) per hectare, at about 1×10⁴ to about 1×10¹⁰ colony forming units (CFU) per hectare, at about 1×10⁴ to about 1×10⁸ colony forming units (CFU) per hectare, at about 1×10⁶ to about 1×10¹⁴ colony forming units (CFU) per hectare, at about 1×10⁶ to about 1×10¹² colony forming units (CFU) per hectare, at about 1×10⁶ to about 1×10¹⁰ colony forming units (CFU) per hectare, at about 1×10⁶ to about 1×10⁸ colony forming units (CFU) per hectare, at about 1×10⁸ to about 1×10¹⁴ colony forming units (CFU) per hectare, at about 1×10⁸ to about 1×10¹² colony forming units (CFU) per hectare, or at about 1×10⁸ to about 1×10¹⁰ colony forming units (CFU) per hectare.

In other aspects, the compositions of the present invention are applied at about 1×10⁶ to about 1×10¹⁴ colony forming units (CFU) per hectare, at about 1×10⁶ to about 1×10¹² colony forming units (CFU) per hectare, at about 1×10⁶ to about 1×10¹⁰ colony forming units (CFU) per hectare, at about 1×10⁶ to about 1×10⁸ colony forming units (CFU) per hectare. In yet other aspects, the compositions of the present invention are applied at about 1×10⁹ to about 1×10¹³ colony forming units (CFU) per hectare. In one aspect, the compositions of the present invention are applied at about 1×10¹⁰ to about 1×10¹² colony forming units (CFU) per hectare.

In certain embodiments, the compositions of the present invention are applied at about 0.1 kg to about 20 kg fermentation solids per hectare. In some embodiments, the compositions of the present invention are applied at about 0.1 kg to about 10 kg fermentation solids per hectare. In other embodiments, the compositions of the present invention are applied at about 0.25 kg to about 7.5 kg fermentation solids per hectare. In yet other embodiments, the compositions of the present invention are applied at about 0.5 kg to about 5 kg fermentation solids per hectare. The compositions of the present invention may also be applied at about 1 kg or about 2 kg fermentation solids per hectare.

### DEPOSIT INFORMATION

Samples of the Bacillus thuringiensis strains of the invention have been deposited with the Agricultural Research Service Culture Collection located at the National Center for Agricultural Utilization Research, Agricultural Research Service, U.S. Department of Agriculture (NRRL), 1815 North University Street, Peoria, Ill. 61604, U.S.A., under the Budapest Treaty. Bacillus thuringiensis strains NRRL B-67685, NRRL B-67687, and NRRL B-67688 were deposited on Sep. 26, 2018.

The Bacillus thuringiensis strains have been deposited under conditions that assure that access to the culture will be available during the pendency of this patent application to one determined by the Commissioner of Patents and Trademarks to be entitled thereto under 37 C.F.R. § 1.14 and 35 U.S.C. § 122. However, it should be understood that the availability of a deposit does not constitute a license to practice the subject invention in derogation of patent rights granted by governmental action.

The following examples are given for purely illustrative and non-limiting purposes of the present invention.

### EXAMPLES

### Example 1. Relative Quantification of Zwittermicin a Production in Thirty-Nine Bacillus thuringiensis Strains

While zwittermicin A has little or no observable insecticidal activity on its own, addition of zwittermicin A to Bacillus thuringiensis culture broths significantly increases their activity against Lepidopteran pests (Broderick, N E et al., 2000. Environ. Entomol. 29(1):101-107). A zwittermicin A gene cluster has been identified in Bacillus thuringiensis subs. kurstaki strain HD1 suggesting that certain strains may possess enhanced insecticidal activity resulting from their biosynthesis of this compound (Nair, J R et al., 2004. J. Appl. Microbiol. 97:495-503). To identify zwittermicin A-producing strains of Bacillus thuringiensis thirty-nine strains were cultured in a soy-based medium. The whole broths from each strain were chemically derivatized to facilitate quantification of zwittermicin A, which was analyzed in each strain with Ultra High Performance Liquid Chromatography/Mass Spectroscopy (UPLC-MS).

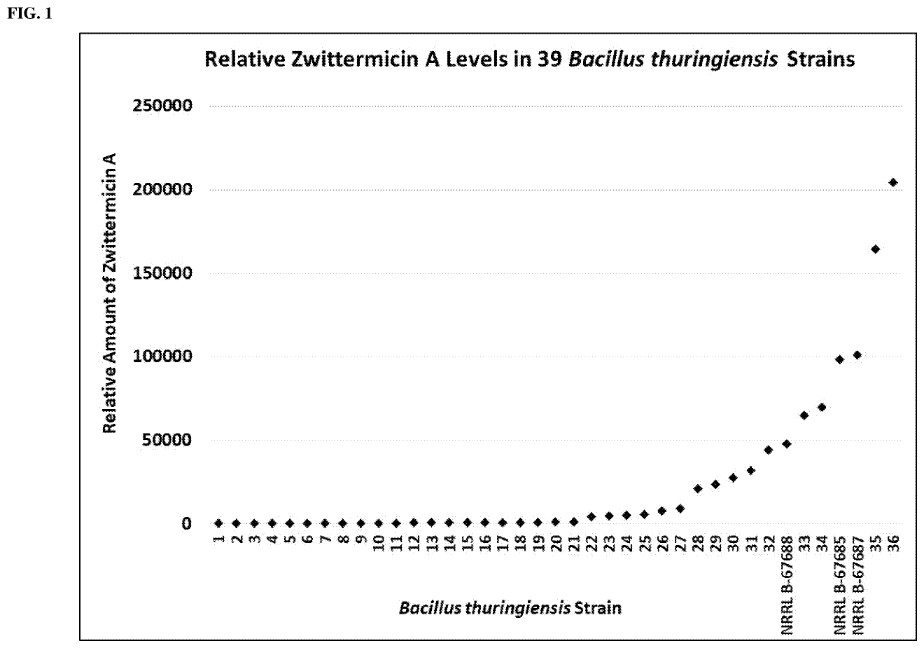

Relative quantities of zwittermicin A in each of the thirty-nine strains are shown in FIG. 1. Bacillus thuringiensis strains NRRL B-67685, NRRL B-67687, and NRRL B-67688 were among the top producers of zwittermicin A.

### Example 2. Zwittermicin a Production in Bacillus thuringiensis Strains NRRL B-67685, NRRL B-67687, and NRRL B-67688 Compared to Commercial Strains

Analysis of relative quantities of zwittermicin A in Bacillus thuringiensis strains NRRL B-67685, NRRL B-67687, and NRRL B-67688 was also compared to the following commercial strains: DELIVER® (Bacillus thuringiensis subspecies kurstaki strain SA-12); DIPEL® (Bacillus thuringiensis subsp. kurstaki strain HD1); JAVELIN® (Bacillus thuringiensis subspecies kurstaki strain SA-11); AGREE® (Bacillus thuringiensis subspecies aizawai strain GC-91); XENTARI® (Bacillus thuringiensis subsp. aizawai strain ABTS-1857); and CRYMAX® (Bacillus thuringiensis subsp. kurstaki strain EG7841). Several of the strains of analyzed Bacillus thuringiensis in Example 1 were included in this analysis as well.

Each strain of Bacillus thuringiensis was cultured in the soy-based medium to produce a whole broth. Each whole broth was then analyzed with Ultra High Performance Liquid Chromatography/Mass Spectroscopy (UHPLC-MS). The signal intensities produced by the zwittermicin A ions in the mass spectrometer from equivalent starting amounts from each whole broth were used to determine relative amounts of zwittermicin A in each strain. The amounts were normalized by the amount of zwittermicin A in CRYMAX® (Bacillus thuringiensis subsp. kurstaki strain EG7841) or in XENTARI® (Bacillus thuringiensis subsp. aizawai strain ABTS-1857) to facilitate comparison.

The results of the analysis are presented in Table 1. These results indicate that relative to the amount in CRYMAX® (Bacillus thuringiensis subsp. kurstaki strain EG7841), Bacillus thuringiensis strains NRRL B-67685, NRRL B-67687, and NRRL B-67688 have at least 25-fold more zwittermicin A, and these four strains have at least 5-fold more zwittermicin A relative to the amount in XENTARI® (Bacillus thuringiensis subsp. aizawai strain ABTS-1857).

TABLE 1
Relative amounts of zwittermicin A in
Bacillus thuringiensis strains.

| | | Zwittermicin A | Zwittermicin A |
|---|---|---|---|
| Bacillus | Relative | Normalized to | Normalized to |
| thuringiensis | Amount of | Amount in | Amount in |
| strain | Zwittermicin A | CRYMAX ® | XENTARI ® |
| Strain 1 | 2457 | 0.85 | 0.22 |
| Strain 2 | 2697 | 0.93 | 0.24 |
| CRYMAX ® | 2891 | 1.00 | 0.26 |
| XENTARI ® | 11121 | 3.85 | 1.00 |
| Strain 3 | 12679 | 4.39 | 1.14 |
| Strain 4 | 17316 | 5.99 | 1.56 |
| Strain 5 | 18893 | 6.54 | 1.70 |
| Strain 6 | 39583 | 13.69 | 3.56 |
| Strain 7 | 49442 | 17.10 | 4.45 |
| DIPEL ® | 79184 | 27.39 | 7.12 |
| NRRL B-67688 | 81801 | 28.30 | 7.36 |
| AGREE ® | 99492 | 34.42 | 8.95 |
| Strain 8 | 102379 | 35.42 | 9.21 |
| NRRL B-67685 | 103527 | 35.82 | 9.31 |
| Strain 9 | 121837 | 42.15 | 10.96 |
| JAVELIN ® | 121852 | 42.16 | 10.96 |
| Strain 10 | 123170 | 42.61 | 11.08 |
| Strain 11 | 125280 | 43.34 | 11.27 |
| NRRL B-67687 | 150730 | 52.15 | 13.55 |
| DELIVER ® | 201135 | 69.58 | 18.09 |

### Example 3. Analysis of Insecticidal Toxin Genes in Bacillus thuringiensis Strains NRRL B-67685, NRRL B-67687, and NRRL B-67688

The genomes of Bacillus thuringiensis strains NRRL B-67685, NRRL B-67687, and NRRL B-67688 were sequenced and analyzed for the presence of known insecticidal toxin genes. This analysis revealed that each of the three strains share 100% sequence identity for the insecticidal toxin genes Vip3Aa7, Cry1Ia2, and Cry2Ab1 (see Table 2 for the nucleotide sequences and Table 4 for the corresponding amino acid sequences). The three strains also share at least 99.9% sequence identity for the insecticidal toxin genes Cry1Aa3 and Cry1Ab1 (see Table 3 for the nucleotide sequences and Table 5 for the corresponding amino acid sequences).

TABLE 2
Nucleotide sequences of genes encoding insecticidal toxins
in which Bacillus thuringiensis strains NRRL B-67685, NRRL B-67687,
and NRRL B-67688 share 100% sequence identity.

| | SEQ ID | |
|---|---|---|
| Gene | NO: | Sequence |
| Vip3Aa7 | 1 | ATGAACAAGAATAATACTAAATTAAGCACAAGAGCCTTACCAAGTTTTATTGATTATTTTAATGGCATTTAT |
| | | GGATTTGCCACTGGTATCAAAGACATTATGAACATGATTTTTAAAACGGATACAGGTGGTGATCTAACCCTA |
| | | GACGAAATTTTAAAGAATCAGCAGTTACTAAATGATATTTCTGGTAAATTGGATGGGGTGAATGGAAGCTT |
| | | AAATGATCTTATCGCACAGGGAAACTTAAATACAGAATTATCTAAGGAAATATTAAAAATTGCAAATGAAC |
| | | AAAATCAAGTTTTAAATGATGTTAATAACAAACTCGATGCGATAAATACGATGCTTCGGGTATATCTACCTA |
| | | AAATTACCTCTATGTTGAGTGATGTAATGAAACAAAATTATGCGCTAAGTCTGCAAATAGAATACTTAAGTA |
| | | AACAATTGCAAGAGATTTCTGATAAGTTGGATATTATTAATGTAAATGTACTTATTAACTCTACACTTACTG |
| | | AAATTACACCTGCGTATCAAAGGATTAAATATGTGAACGAAAAATTTGAGGAATTAACTTTTGCTACAGAA |
| | | ACTAGTTCAAAAGTAAAAAAGGATGGCTCTCCTGCAGATATTCTTGATGAGTTAACTGAGTTAACTGAACTA |
| | | GCGAAAAGTGTAACAAAAAATGATGTGGATGGTTTTGAATTTTACCTTAATACATTCCACGATGTAATGGTA |
| | | GGAAATAATTTATTCGGGCGTTCAGCTTTAAAAACTGCATCGGAATTAATTACTAAAGAAAATGTGAAAAC |
| | | AAGTGGCAGTGAGGTCGGAAATGTTTATAACTTCTTAATTGTATTAACAGCTCTGCAAGCAAAAGCTTTTCT |
| | | TACTTTAACAACATGCCGAAAATTATTAGGCTTAGCAGATATTGATTATACTTCTATTATGAATGAACATTTA |
| | | AATAAGGAAAAAGAGGAATTTAGAGTAAACATCCTCCCTACACTTTCTAATACTTTTTCTAATCCTAATTAT |
| | | GCAAAAGTTAAAGGAAGTGATGAAGATGCAAAGATGATTGTGGAAGCTAAACCAGGACATGCATTGATTG |
| | | GGTTTGAAATTAGTAATGATTCAATTACAGTATTAAAAGTATATGAGGCTAAGCTAAAACAAAATTATCAA |
| | | GTCGATAAGGATTCCTTATCGGAAGTTATTTATGGTGATATGGATAAATTATTGTGCCCAGATCAATCTGAA |
| | | CAAATCTATTATACAAATAACATAGTATTTCCAAATGAATATGTAATTACTAAAATTGATTTCACTAAAAAA |
| | | ATGAAAACTTTAAGATATGAGGTAACAGCGAATTTTTATGATTCTTCTACAGGAGAAATTGACTTAAATAAG |
| | | AAAAAAGTAGAATCAAGTGAAGCGGAGTATAGAACGTTAAGTGCTAATGATGATGGGGTGTATATGCCGTT |
| | | AGGTGTCATCAGTGAAACATTTTTGACTCCGATTAATGGGTTTGGCCTCCAAGCTGATGAAAATTCAAGATT |
| | | AATTACTTTAACATGTAAATCATATTTAAGAGAACTACTGCTAGCAACAGACTTAAGCAATAAAGAAACTA |
| | | AATTGATCGTCCCGCCAAGTGGTTTTATTAGCAATATTGTAGAGAACGGGTCCATAGAAGAGGACAATTTA |
| | | GAGCCGTGGAAAGCAAATAATAAGAATGCGTATGTAGATCATACAGGCGGAGTGAATGGAACTAAAGCTTT |
| | | ATATGTTCATAAGGACGGAGGAATTTCACAATTTATTGGAGATAAGTTAAAACCGAAAACTGAGTATGTAA |
| | | TCCAATATACTGTTAAAGGAAAACCTTCTATTCATTTAAAAGATGAAAATACTGGATATATTCATTATGAAG |
| | | ATACAAATAATAATTTAGAAGATTATCAAACTATTAATAAACGTTTTACTACAGGAACTGATTTAAAGGGA |
| | | GTGTATTTAATTTTAAAAAGTCAAAATGGAGATGAAGCTTGGGGAGATAACTTTATTATTTTGGAAATTAGT |
| | | CCTTCTGAAAAGTTATTAAGTCCAGAATTAATTAATACAAATAATTGGACGAGTACGGGATCAACTAATATT |
| | | AGCGGTAATACACTCACTCTTTATCAGGGAGGACGAGGGATTCTAAAACAAAACCTTCAATTAGATAGTTTT |
| | | TCAACTTATAGAGTGTATTTTTCTGTGTCCGGAGATGCTAATGTAAGGATTAGAAATTCTAGGGAAGTGTTA |
| | | TTTGAAAAAAGATATATGAGCGGTGCTAAAGATGTTTCTGAAATGTTCACTACAAAATTTGAGAAAGATAA |
| | | CTTTTATATAGAGCTTTCTCAAGGGAATAATTTATATGGTGGTCCTATTGTACATTTTTACGATGTCTCTATT |
| | | AAGTAA |
| Cry1Ia2 | 2 | ATGAAACTAAAGAATCAAGATAAGCATCAAAGTTTTTCTAGCAATGCGAAAGTAGATAAAATCTCTACGGA |
| | | TTCACTAAAAAATGAAACAGATATAGAATTACAAAACATTAATCATGAAGATTGTTTGAAAATGTCTGAGT |
| | | ATGAAAATGTAGAGCCGTTTGTTAGTGCATCAACAATTCAAACAGGTATTGGTATTGCGGGTAAAATACTTG |
| | | GTACCCTAGGCGTTCCTTTTGCAGGACAAGTAGCTAGTCTTTATAGTTTTATCTTAGGTGAGCTATGGCCTAA |
| | | GGGGAAAAATCAATGGGAAATCTTTATGGAACATGTAGAAGAGATTATTAATCAAAAAATATCAACTTATG |
| | | CAAGAAATAAAGCACTTACAGACTTGAAAGGATTAGGAGATGCCTTAGCTGTCTACCATGATTCGCTTGAA |
| | | AGTTGGGTTGGAAATCGTAATAACACAAGGGCTAGGAGTGTTGTCAAGAGCCAATATATCGCATTAGAATT |
| | | GATGTTCGTTCAGAAACTACCTTCTTTTGCAGTGTCTGGAGAGGAGGTACCATTATTACCGATATATGCCCA |
| | | AGCTGCAAATTTACATTTGTTGCTATTAAGAGATGCATCTATTTTTGGAAAAGAGTGGGGATTATCATCTTC |
| | | AGAAATTTCAACATTTTATAACCGTCAAGTCGAACGAGCAGGAGATTATTCCGACCATTGTGTGAAATGGTA |
| | | TAGCACAGGTCTAAATAACTTGAGGGGTACAAATGCCGAAAGTTGGGTACGATATAATCAATTCCGTAGAG |
| | | ACATGACTTTAATGGTACTAGATTTAGTGGCACTATTTCCAAGCTATGATACACAAATGTATCCAATTAAAA |
| | | CTACAGCCCAACTTACAAGAGAAGTATATACAGACGCAATTGGGACAGTACATCCGCATCCAAGTTTTACA |
| | | AGTACGACTTGGTATAATAATAATGCACCTTCGTTCTCTGCCATAGAGGCTGCTGTTGTTCGAAACCCGCAT |
| | | CTACTCGATTTTCTAGAACAAGTTACAATTTACAGCTTATTAAGTCGATGGAGTAACACTCAGTATATGAAT |
| | | ATGTGGGGAGGACATAAACTAGAATTCCGAACAATAGGAGGAACGTTAAATATCTCAACACAAGGATCTAC |
| | | TAATACTTCTATTAATCCTGTAACATTACCGTTCACTTCTCGAGACGTCTATAGGACTGAATCATTGGCAGGG |
| | | CTGAATCTATTTTTAACTCAACCTGTTAATGGAGTACCTAGGGTTGATTTTCATTGGAAATTCGTCACACATC |
| | | CGATCGCATCTGATAATTTCTATTATCCAGGGTATGCTGGAATTGGGACGCAATTACAGGATTCAGAAAATG |
| | | AATTACCACCTGAAGCAACAGGACAGCCAAATTATGAATCTTATAGTCATAGATTATCTCATATAGGACTCA |
| | | TTTCAGCATCACATGTGAAAGCATTGGTATATTCTTGGACGCATCGTAGTGCAGATCGTACAAATACAATTG |
| | | AGCCAAATAGCATTACACAAATACCATTAGTAAAAGCTTTCAATCTGTCTTCAGGTGCCGCTGTAGTGAGAG |
| | | GACCAGGATTTACAGGTGGGGATATCCTTCGAAGAACGAATACTGGTACATTTGGGGATATACGAGTAAAT |
| | | ATTAATCCACCATTTGCACAAAGATATCGCGTGAGGATTCGCTATGCTTCTACCACAGATTTACAATTCCAT |
| | | ACGTCAATTAACGGTAAAGCTATTAATCAAGGTAATTTTTCAGCAACTATGAATAGAGGAGAGGACTTAGA |
| | | CTATAAAACCTTTAGAACTGTAGGCTTTACCACTCCATTTAGCTTTTTAGATGTACAAAGTACATTCACAATA |
| | | GGTGCTTGGAACTTCTCTTCAGGTAACGAAGTTTATATAGATAGAATTGAATTTGTTCCGGTAGAAGTAACA |
| | | TATGAGGCAGAATATGATTTTGAAAAAGCGCAAGAGAAGGTTACTGCACTGTTTACATCTACGAATCCAAG |
| | | AGGATTAAAAACAGATGTAAAGGATTATCATATTGACCAGGTATCAAATTTAGTAGAGTCTCTATCAGATG |
| | | AATTCTATCTTGATGAAAAGAGAGAATTATTCGAGATAGTTAAATACGCGAAGCAACTCCATATTGAGCGT |
| | | AACATGTAG |
| Cry2Ab1 | 3 | ATGAATAGTGTATTGAATAGCGGAAGAACTACTATTTGTGATGCGTATAATGTAGCGGCTCATGATCCATTT |
| | | AGTTTTCAACACAAATCATTAGATACCGTACAAAAGGAATGGACGGAGTGGAAAAAAAATAATCATAGTTT |
| | | ATACCTAGATCCTATTGTTGGAACTGTGGCTAGTTTTCTGTTAAAGAAAGTGGGGAGTCTTGTTGGAAAAAG |
| | | GATACTAAGTGAGTTACGGAATTTAATATTTCCTAGTGGTAGTACAAATCTAATGCAAGATATTTTAAGAGA |
| | | GACAGAAAAATTCCTGAATCAAAGACTTAATACAGACACTCTTGCCCGTGTAAATGCGGAATTGACAGGGC |
| | | TGCAAGCAAATGTAGAAGAGTTTAATCGACAAGTAGATAATTTTTTGAACCCTAACCGAAACGCTGTTCCTT |
| | | TATCAATAACTTCTTCAGTTAATACAATGCAACAATTATTTCTAAATAGATTACCCCAGTTCCAGATGCAAG |
| | | GATACCAACTGTTATTATTACCTTTATTTGCACAGGCAGCCAATTTACATCTTTCTTTTATTAGAGATGTTATT |
| | | CTAAATGCAGATGAATGGGGAATTTCAGCAGCAACATTACGTACGTATCGAGATTACTTGAAAAATTATAC |
| | | AAGAGATTACTCTAACTATTGTATAAATACGTATCAAAGTGCGTTTAAAGGTTTAAACACTCGTTTACACGA |
| | | TATGTTAGAATTTAGAACATATATGTTTTTAAATGTATTTGAGTATGTATCTATCTGGTCGTTGTTTAAATAT |
| | | CAAAGTCTTCTAGTATCTTCCGGTGCTAATTTATATGCAAGTGGTAGTGGACCACAGCAGACCCAATCATTT |
| | | ACTTCACAAGACTGGCCATTTTTATATTCTCTTTTCCAAGTTAATTCAAATTATGTGTTAAATGGATTTAGTG |
| | | GTGCTAGGCTTTCTAATACCTTCCCTAATATAGTTGGTTTACCTGGTTCTACTACAACTCACGCATTGCTTGC |
| | | TGCAAGGGTTAATTACAGTGGAGGAATTTCGTCTGGTGATATAGGTGCATCTCCGTTTAATCAAAATTTTAA |
| | | TTGTAGCACATTTCTCCCCCCATTGTTAACGCCATTTGTTAGGAGTTGGCTAGATTCAGGTTCAGATCGGGAG |
| | | GGCGTTGCCACCGTTACAAATTGGCAAACAGAATCCTTTGAGACAACTTTAGGGTTAAGGAGTGGTGCTTTT |
| | | ACAGCTCGCGGTAATTCAAACTATTTCCCAGATTATTTTATTCGTAATATTTCTGGAGTTCCTTTAGTTGTTA |
| | | GAAATGAAGATTTAAGAAGACCGTTACACTATAATGAAATAAGAAATATAGCAAGTCCTTCAGGAACACCT |
| | | GGTGGAGCACGAGCTTATATGGTATCTGTGCATAACAGAAAAAATAATATCCATGCTGTTCATGAAAATGG |
| | | TTCTATGATTCATTTAGCGCCAAATGACTATACAGGATTTACTATTTCGCCGATACATGCAACTCAAGTGAA |
| | | TAATCAAACACGAACATTTATTTCTGAAAAATTTGGAAATCAAGGTGATTCTTTAAGGTTTGAACAAAACAA |
| | | CACGACAGCTCGTTATACGCTTAGAGGGAATGGAAATAGTTACAATCTTTATTTAAGAGTTTCTTCAATAGG |
| | | AAATTCCACTATTCGAGTTACTATAAACGGTAGGGTATATACTGCTACAAATGTTAATACTACTACAAATAA |
| | | CGATGGAGTTAATGATAATGGAGCTCGTTTTTCAGATATTAATATCGGTAATGTAGTAGCAAGTAGTAATTC |
| | | TGATGTACCATTAGATATAAATGTAACATTAAACTCCGGTACTCAATTTGATCTTATGAATATTATGCTTGTA |
| | | CCAACTAATATTTCACCACTTTATTAA |

TABLE 3
Nucleotide sequences of genes encoding insecticidal toxins
in which Bacillus thuringiensis strains NRRL B-67685, NRRL B-67687,
and NRRL B-67688 share at least 99.9% sequence identity.

| | | SEQ ID | |
|---|---|---|---|
| Strain(s) | Gene | NO: | Sequence |
| NRRL B- | Cry1Aa8 | 4 | ATGGATAACAATCCGAACATCAATGAATGCATTCCTTATAATTGTTTAAGTAACCCTGAAGTAGA |
| 67687 & | | | AGTATTAGGTGGAGAAAGAATAGAAACTGGTTACACCCCAATCGATATTTCCTTGTCGCTAACG |
| NRRL B- | | | CAATTTCTTTTGAGTGAATTTGTTCCCGGTGCTGGATTTGTGTTAGGACTAGTTGATATAATATGG |
| 67688 | | | GGAATTTTTGGTCCCTCTCAATGGGACGCATTTCTTGTACAAATTGAACAGTTAATTAACCAAAG |
| | | | AATAGAAGAATTCGCTAGGAACCAAGCCATTTCTAGATTAGAAGGACTAAGCAATCTTTATCAA |
| | | | ATTTACGCAGAATCTTTTAGAGAGTGGGAAGCAGATCCTACTAATCCAGCATTAAGAGAAGAGA |
| | | | TGCGTATTCAATTCAATGACATGAACAGTGCCCTTACAACCGCTATTCCTCTTTTGGCAGTTCAA |
| | | | AATTATCAAGTTCCTCTTTTATCAGTATATGTTCAAGCTGCAAATTTACATTTATCAGTTTTGAGA |
| | | | GATGTTTCAGTGTTTGGACAAAGGTGGGGATTTGATGCCGCGACTATCAATAGTCGTTATAATGA |
| | | | TTTAACTAGGCTTATTGGCAACTATACAGATTATGCTGTGCGCTGGTACAATACGGGATTAGAGC |
| | | | GTGTATGGGGACCGGATTCTAGAGATTGGGTAAGGTATAATCAATTTAGAAGAGAGCTAACACT |
| | | | TACTGTATTAGATATCGTTGCTCTATTCTCAAATTATGATAGTCGAAGGTATCCAATTCGAACAG |
| | | | TTTCCCAATTAACAAGAGAAATTTATACGAACCCAGTATTAGAAAATTTTGATGGTAGTTTTCGT |
| | | | GGAATGGCTCAGAGAATAGAACAGAATATTAGGCAACCACATCTTATGGATATCCTTAATAGTA |
| | | | TAACCATTTATACTGATGTGCATAGAGGCTTTAATTATTGGTCAGGGCATCAAATAACAGCTTCT |
| | | | CCTGTAGGGTTTTCAGGACCAGAATTCGCATTCCCTTTATTTGGGAATGCGGGGAATGCAGCTCC |
| | | | ACCCGTACTTGTCTCATTAACTGGTTTGGGGATTTTTAGAACATTATCTTCACCTTTATATAGAAG |
| | | | AATTATACTTGGTTCAGGCCCAAATAATCAGGAACTGTTTGTCCTTGATGGAACGGAGTTTTCTT |
| | | | TTGCCTCCCTAACGACCAACTTGCCTTCCACTATATATAGACAAAGGGGTACAGTCGATTCACTA |
| | | | GATGTAATACCGCCACAGGATAATAGTGTACCACCTCGTGCGGGATTTAGCCATCGATTGAGTC |
| | | | ATGTTACAATGCTGAGCCAAGCAGCTGGAGCAGTTTACACCTTGAGAGCTCCAACGTTTTCTTGG |
| | | | CAGCATCGCAGTGCTGAATTTAATAATATAATTCCTTCATCACAAATTACACAAATACCTTTAAC |
| | | | AAAATCTACTAATCTTGGCTCTGGAACTTCTGTCGTTAAAGGACCAGGATTTACAGGAGGAGAT |
| | | | ATTCTTCGAAGAACTTCACCTGGCCAGATTTCAACCTTAAGAGTAAATATTACTGCACCATTATC |
| | | | ACAAAGATATCGGGTAAGAATTCGCTACGCTTCTACTACAAATTTACAATTCCATACATCAATTG |
| | | | ACGGAAGACCTATTAATCAGGGTAATTTTTCAGCAACTATGAGTAGTGGGAGTAATTTACAGTC |
| | | | CGGAAGCTTTAGGACTGTAGGTTTTACTACTCCGTTTAACTTTTCAAATGGATCAAGTGTATTTA |
| | | | CGTTAAGTGCTCATGTCTTCAATTCAGGCAATGAAGTTTATATAGATCGAATTGAATTTGTTCCG |
| | | | GCAGAAGTAACCTTTGAGGCAGAATATGATTTAGAAAGAGCACAAAAGGCGGTGAATGAGCTG |
| | | | TTTACTTCTTCCAATCAAATCGGGTTAAAAACAGATGTGACGGATTATCATATTGATCAAGTATC |
| | | | CAATTTAGTTGAGTGTTTATCAGATGAATTTTGTCTGGATGAAAAACAAGAATTGTCCGAGAAA |
| | | | GTCAAACATGCGAAGCGACTTAGTGATGAGCGGAATTTACTTCAAGATCCAAACTTCAGAGGGA |
| | | | TCAATAGACAACTAGACCGTGGCTGGAGAGGAAGTACGGATATTACCATCCAAGGAGGCGATG |
| | | | ACGTATTCAAAGAGAATTACGTTACGCTATTGGGTACCTTTGATGAGTGCTATCCAACGTATTTA |
| | | | TATCAAAAAATAGATGAGTCGAAATTAAAAGCCTATACCCGTTATCAATTAAGAGGGTATATCG |
| | | | AAGATAGTCAAGACTTAGAAATCTATTTAATTCGCTACAATGCAAAACATGAAACAGTAAATGT |
| | | | GCCAGGTACGGGTTCCTTATGGCCGCTTTCAGCCCAAAGTCCAATCGGAAAGTGTGGAGAGCCG |
| | | | AATCGATGCGCGCCACACCTTGAATGGAATCCTGACTTAGATTGTTCGTGTAGGGATGGAGAAA |
| | | | AGTGTGCCCATCATTCGCATCATTTCTCCTTAGACATTGATGTAGGATGTACAGACTTAAATGAG |
| | | | GACCTAGGTGTATGGGTGATCTTTAAGATTAAGACGCAAGATGGGCACGCAAGACTAGGGAATC |
| | | | TAGAGTTTCTCGAAGAGAAACCATTAGTAGGAGAAGCGCTAGCTCGTGTGAAAAGAGCGGAGA |
| | | | AAAAATGGAGAGACAAACGTGAAAAATTGGAATGGGAAACAAATATCGTTTATAAAGAGGCAA |
| | | | AAGAATCTGTAGATGCTTTATTTGTAAACTCTCAATATGATCAATTACAAGCGGATACGAATATT |
| | | | GCCATGATTCATGCGGCAGATAAACGTGTTCATAGCATTCGAGAAGCTTATCTGCCTGAGCTGTC |
| | | | TGTGATTCCGGGTGTCAATGCGGCTATTTTTGAAGAATTAGAAGGGCGTATTTTCACTGCATTCT |
| | | | CCCTATATGATGCGAGAAATGTCATTAAAAATGGTGATTTTAATAATGGCTTATCCTGCTGGAAC |
| | | | GTGAAAGGGCATGTAGATGTAGAAGAACAAAACAACCAACGTTCGGTCCTTGTTGTTCCGGAAT |
| | | | GGGAAGCAGAAGTGTCACAAGAAGTTCGTGTCTGTCCGGGTCGTGGCTATATCCTTCGTGTCAC |
| | | | AGCGTACAAGGAGGGATATGGAGAAGGTTGCGTAACCATTCATGAGATCGAGAACAATACAGA |
| | | | CGAACTGAAGTTTAGCAACTGCGTAGAAGAGGAAATCTATCCAAATAACACGGTAACGTGTAAT |
| | | | GATTATACTGTAAATCAAGAAGAATACGGAGGTGCGTACACTTCTCGTAATCGAGGATATAACG |
| | | | AAGCTCCTTCCGTACCAGCTGATTATGCGTCAGTCTATGAAGAAAAATCGTATACAGATGGACG |
| | | | AAGAGAGAATCCTTGTGAATTTAACAGAGGGTATAGGGATTACACGCCACTACCAGTTGGTTAT |
| | | | GTGACAAAAGAATTAGAATACTTCCCAGAAACCGATAAGGTATGGATTGAGATTGGAGAAACG |
| | | | GAAGGAACATTTATCGTGGACAGCGTGGAATTACTCCTTATGGAGGAATAG |
| NRRL B- | Cry1Aa3 | 5 | ATGGATAACAATCCGAACATCAATGAATGCATTCCTTATAATTGTTTAAGTAACCCTGAAGTAGA |
| 67685 | | | AGTATTAGGTGGAGAAAGAATAGAAACTGGTTACACCCCAATCGATATTTCCTTGTCGCTAACG |
| | | | CAATTTCTTTTGAGTGAATTTGTTCCCGGTGCTGGATTTGTGTTAGGACTAGTTGATATAATATGG |
| | | | GGAATTTTTGGTCCCTCTCAATGGGACGCATTTCTTGTACAAATTGAACAGTTAATTAACCAAAG |
| | | | AATAGAAGAATTCGCTAGGAACCAAGCCATTTCTAGATTAGAAGGACTAAGCAATCTTTATCAA |
| | | | ATTTACGCAGAATCTTTTAGAGAGTGGGAAGCAGATCCTACTAATCCAGCATTAAGAGAAGAGA |
| | | | TGCGTATTCAATTCAATGACATGAACAGTGCCCTTACAACCGCTATTCCTCTTTTTGCAGTTCAA |
| | | | AATTATCAAGTTCCTCTTTTATCAGTATATGTTCAAGCTGCAAATTTACATTTATCAGTTTTGAGA |
| | | | GATGTTTCAGTGTTTGGACAAAGGTGGGGATTTGATGCCGCGACTATCAATAGTCGTTATAATGA |
| | | | TTTAACTAGGCTTATTGGCAACTATACAGATTATGCTGTGCGCTGGTACAATACGGGATTAGAGC |
| | | | GTGTATGGGGACCGGATTCTAGAGATTGGGTAAGGTATAATCAATTTAGAAGAGAGCTAACACT |
| | | | TACTGTATTAGATATCGTTGCTCTATTCTCAAATTATGATAGTCGAAGGTATCCAATTCGAACAG |
| | | | TTTCCCAATTAACAAGAGAAATTTATACGAACCCAGTATTAGAAAATTTTGATGGTAGTTTTCGT |
| | | | GGAATGGCTCAGAGAATAGAACAGAATATTAGGCAACCACATCTTATGGATATCCTTAATAGTA |
| | | | TAACCATTTATACTGATGTGCATAGAGGCTTTAATTATTGGTCAGGGCATCAAATAACAGCTTCT |
| | | | CCTGTAGGGTTTTCAGGACCAGAATTCGCATTCCCTTTATTTGGGAATGCGGGGAATGCAGCTCC |
| | | | ACCCGTACTTGTCTCATTAACTGGTTTGGGGATTTTTAGAACATTATCTTCACCTTTATATAGAAG |
| | | | AATTATACTTGGTTCAGGCCCAAATAATCAGGAACTGTTTGTCCTTGATGGAACGGAGTTTTCTT |
| | | | TTGCCTCCCTAACGACCAACTTGCCTTCCACTATATATAGACAAAGGGGTACAGTCGATTCACTA |
| | | | GATGTAATACCGCCACAGGATAATAGTGTACCACCTCGTGCGGGATTTAGCCATCGATTGAGTC |
| | | | ATGTTACAATGCTGAGCCAAGCAGCTGGAGCAGTTTACACCTTGAGAGCTCCAACGTTTTCTTGG |
| | | | CAGCATCGCAGTGCTGAATTTAATAATATAATTCCTTCATCACAAATTACACAAATACCTTTAAC |
| | | | AAAATCTACTAATCTTGGCTCTGGAACTTCTGTCGTTAAAGGACCAGGATTTACAGGAGGAGAT |
| | | | ATTCTTCGAAGAACTTCACCTGGCCAGATTTCAACCTTAAGAGTAAATATTACTGCACCATTATC |
| | | | ACAAAGATATCGGGTAAGAATTCGCTACGCTTCTACTACAAATTTACAATTCCATACATCAATTG |
| | | | ACGGAAGACCTATTAATCAGGGTAATTTTTCAGCAACTATGAGTAGTGGGAGTAATTTACAGTC |
| | | | CGGAAGCTTTAGGACTGTAGGTTTTACTACTCCGTTTAACTTTTCAAATGGATCAAGTGTATTTA |
| | | | CGTTAAGTGCTCATGTCTTCAATTCAGGCAATGAAGTTTATATAGATCGAATTGAATTTGTTCCG |
| | | | GCAGAAGTAACCTTTGAGGCAGAATATGATTTAGAAAGAGCACAAAAGGCGGTGAATGAGCTG |
| | | | TTTACTTCTTCCAATCAAATCGGGTTAAAAACAGATGTGACGGATTATCATATTGATCAAGTATC |
| | | | CAATTTAGTTGAGTGTTTATCAGATGAATTTTGTCTGGATGAAAAACAAGAATTGTCCGAGAAA |
| | | | GTCAAACATGCGAAGCGACTTAGTGATGAGCGGAATTTACTTCAAGATCCAAACTTCAGAGGGA |
| | | | TCAATAGACAACTAGACCGTGGCTGGAGAGGAAGTACGGATATTACCATCCAAGGAGGCGATG |
| | | | ACGTATTCAAAGAGAATTACGTTACGCTATTGGGTACCTTTGATGAGTGCTATCCAACGTATTTA |
| | | | TATCAAAAAATAGATGAGTCGAAATTAAAAGCCTATACCCGTTATCAATTAAGAGGGTATATCG |
| | | | AAGATAGTCAAGACTTAGAAATCTATTTAATTCGCTACAATGCAAAACATGAAACAGTAAATGT |
| | | | GCCAGGTACGGGTTCCTTATGGCCGCTTTCAGCCCAAAGTCCAATCGGAAAGTGTGGAGAGCCG |
| | | | AATCGATGCGCGCCACACCTTGAATGGAATCCTGACTTAGATTGTTCGTGTAGGGATGGAGAAA |
| | | | AGTGTGCCCATCATTCGCATCATTTCTCCTTAGACATTGATGTAGGATGTACAGACTTAAATGAG |
| | | | GACCTAGGTGTATGGGTGATCTTTAAGATTAAGACGCAAGATGGGCACGCAAGACTAGGGAATC |
| | | | TAGAGTTTCTCGAAGAGAAACCATTAGTAGGAGAAGCGCTAGCTCGTGTGAAAAGAGCGGAGA |
| | | | AAAAATGGAGAGACAAACGTGAAAAATTGGAATGGGAAACAAATATCGTTTATAAAGAGGCAA |
| | | | AAGAATCTGTAGATGCTTTATTTGTAAACTCTCAATATGATCAATTACAAGCGGATACGAATATT |
| | | | GCCATGATTCATGCGGCAGATAAACGTGTTCATAGCATTCGAGAAGCTTATCTGCCTGAGCTGTC |
| | | | TGTGATTCCGGGTGTCAATGCGGCTATTTTTGAAGAATTAGAAGGGCGTATTTTCACTGCATTCT |
| | | | CCCTATATGATGCGAGAAATGTCATTAAAAATGGTGATTTTAATAATGGCTTATCCTGCTGGAAC |
| | | | GTGAAAGGGCATGTAGATGTAGAAGAACAAAACAACCAACGTTCGGTCCTTGTTGTTCCGGAAT |
| | | | GGGAAGCAGAAGTGTCACAAGAAGTTCGTGTCTGTCCGGGTCGTGGCTATATCCTTCGTGTCAC |
| | | | AGCGTACAAGGAGGGATATGGAGAAGGTTGCGTAACCATTCATGAGATCGAGAACAATACAGA |
| | | | CGAACTGAAGTTTAGCAACTGCGTAGAAGAGGAAATCTATCCAAATAACACGGTAACGTGTAAT |
| | | | GATTATACTGTAAATCAAGAAGAATACGGAGGTGCGTACACTTCTCGTAATCGAGGATATAACG |
| | | | AAGCTCCTTCCGTACCAGCTGATTATGCGTCAGTCTATGAAGAAAAATCGTATACAGATGGACG |
| | | | AAGAGAGAATCCTTGTGAATTTAACAGAGGGTATAGGGATTACACGCCACTACCAGTTGGTTAT |
| | | | GTGACAAAAGAATTAGAATACTTCCCAGAAACCGATAAGGTATGGATTGAGATTGGAGAAACG |
| | | | GAAGGAACATTTATCGTGGACAGCGTGGAATTACTCCTTATGGAGGAATAG |
| NRRL B- | Cry1Ab1 | 6 | ATGGATAACAATCCGAACATCAATGAATGCATTCCTTATAATTGTTTAAGTAACCCTGAAGTAGA |
| 67685 & | | | AGTATTAGGTGGAGAAAGAATAGAAACTGGTTACACCCCAATCGATATTTCCTTGTCGCTAACG |
| NRRL B- | | | CAATTTCTTTTGAGTGAATTTGTTCCCGGTGCTGGATTTGTGTTAGGACTAGTTGATATAATATGG |
| 67687 | | | GGAATTTTTGGTCCCTCTCAATGGGACGCATTTCTTGTACAAATTGAACAGTTAATTAACCAAAG |
| | | | AATAGAAGAATTCGCTAGGAACCAAGCCATTTCTAGATTAGAAGGACTAAGCAATCTTTATCAA |
| | | | ATTTACGCAGAATCTTTTAGAGAGTGGGAAGCAGATCCTACTAATCCAGCATTAAGAGAAGAGA |
| | | | TGCGTATTCAATTCAATGACATGAACAGTGCCCTTACAACCGCTATTCCTCTTTTTGCAGTTCAA |
| | | | AATTATCAAGTTCCTCTTTTATCAGTATATGTTCAAGCTGCAAATTTACATTTATCAGTTTTGAGA |
| | | | GATGTTTCAGTGTTTGGACAAAGGTGGGGATTTGATGCCGCGACTATCAATAGTCGTTATAATGA |
| | | | TTTAACTAGGCTTATTGGCAACTATACAGATCATGCTGTACGCTGGTACAATACGGGATTAGAGC |
| | | | GTGTATGGGGACCGGATTCTAGAGATTGGATAAGATATAATCAATTTAGAAGAGAATTAACACT |
| | | | AACTGTATTAGATATCGTTTCTCTATTTCCGAACTATGATAGTAGAACGTATCCAATTCGAACAG |
| | | | TTTCCCAATTAACAAGAGAAATTTATACAAACCCAGTATTAGAAAATTTTGATGGTAGTTTTCGA |
| | | | GGCTCGGCTCAGGGCATAGAAGGAAGTATTAGGAGTCCACATTTGATGGATATACTTAACAGTA |
| | | | TAACCATCTATACGGATGCTCATAGAGGAGAATATTATTGGTCAGGGCATCAAATAATGGCTTCT |
| | | | CCTGTAGGGTTTTCGGGGCCAGAATTCACTTTTCCGCTATATGGAACTATGGGAAATGCAGCTCC |
| | | | ACAACAACGTATTGTTGCTCAACTAGGTCAGGGCGTGTATAGAACATTATCGTCCACTTTATATA |
| | | | GAAGACCTTTTAATATAGGGATAAATAATCAACAACTATCTGTTCTTGACGGGACAGAATTTGCT |
| | | | TATGGAACCTCCTCAAATTTGCCATCCGCTGTATACAGAAAAAGCGGAACGGTAGATTCGCTGG |
| | | | ATGAAATACCGCCACAGAATAACAACGTGCCACCTAGGCAAGGATTTAGTCATCGATTAAGCCA |
| | | | TGTTTCAATGTTTCGTTCAGGCTTTAGTAATAGTAGTGTAAGTATAATAAGAGCTCCTATGTTCTC |
| | | | TTGGATACATCGTAGTGCTGAATTTAATAATATAATTCCTTCATCACAAATTACACAAATACCTT |
| | | | TAACAAAATCTACTAATCTTGGCTCTGGAACTTCTGTCGTTAAAGGACCAGGATTTACAGGAGG |
| | | | AGATATTCTTCGAAGAACTTCACCTGGCCAGATTTCAACCTTAAGAGTAAATATTACTGCACCAT |
| | | | TATCACAAAGATATCGGGTAAGAATTCGCTACGCTTCTACCACAAATTTACAATTCCATACATCA |
| | | | ATTGACGGAAGACCTATTAATCAGGGGAATTTTTCAGCAACTATGAGTAGTGGGAGTAATTTAC |
| | | | AGTCCGGAAGCTTTAGGACTGTAGGTTTTACTACTCCGTTTAACTTTTCAAATGGATCAAGTGTA |
| | | | TTTACGTTAAGTGCTCATGTCTTCAATTCAGGCAATGAAGTTTATATAGATCGAATTGAATTTGTT |
| | | | CCGGCAGAAGTAACCTTTGAGGCAGAATATGATTTAGAAAGAGCACAAAAGGCGGTGAATGAG |
| | | | CTGTTTACTTCTTCCAATCAAATCGGGTTAAAAACAGATGTGACGGATTATCATATTGATCAAGT |
| | | | ATCCAATTTAGTTGAGTGTTTATCTGATGAATTTTGTCTGGATGAAAAAAAAGAATTGTCCGAGA |
| | | | AAGTCAAACATGCGAAGCGACTTAGTGATGAGCGGAATTTACTTCAAGATCCAAACTTTAGAGG |
| | | | GATCAATAGACAACTAGACCGTGGCTGGAGAGGAAGTACGGATATTACCATCCAAGGAGGCGA |
| | | | TGACGTATTCAAAGAGAATTACGTTACGCTATTGGGTACCTTTGATGAGTGCTATCCAACGTATT |
| | | | TATATCAAAAAATAGATGAGTCGAAATTAAAAGCCTATACCCGTTACCAATTAAGAGGGTATAT |
| | | | CGAAGATAGTCAAGACTTAGAAATCTATTTAATTCGCTACAATGCCAAACACGAAACAGTAAAT |
| | | | GTGCCAGGTACGGGTTCCTTATGGCCGCTTTCAGCCCCAAGTCCAATCGGAAAATGTGCCCATCA |
| | | | TTCCCATCATTTCTCCTTGGACATTGATGTTGGATGTACAGACTTAAATGAGGACTTAGGTGTAT |
| | | | GGGTGATATTCAAGATTAAGACGCAAGATGGCCATGCAAGACTAGGAAATCTAGAATTTCTCGA |
| | | | AGAGAAACCATTAGTAGGAGAAGCACTAGCTCGTGTGAAAAGAGCGGAGAAAAAATGGAGAG |
| | | | ACAAACGTGAAAAATTGGAATGGGAAACAAATATTGTTTATAAAGAGGCAAAAGAATCTGTAG |
| | | | ATGCTTTATTTGTAAACTCTCAATATGATAGATTACAAGCGGATACCAACATCGCGATGATTCAT |
| | | | GCGGCAGATAAACGCGTTCATAGCATTCGAGAAGCTTATCTGCCTGAGCTGTCTGTGATTCCGGG |
| | | | TGTCAATGCGGCTATTTTTGAAGAATTAGAAGGGCGTATTTTCACTGCATTCTCCCTATATGATG |
| | | | CGAGAAATGTCATTAAAAATGGTGATTTTAATAATGGCTTATCCTGCTGGAACGTGAAAGGGCA |
| | | | TGTAGATGTAGAAGAACAAAACAACCACCGTTCGGTCCTTGTTGTTCCGGAATGGGAAGCAGAA |
| | | | GTGTCACAAGAAGTTCGTGTCTGTCCGGGTCGTGGCTATATCCTTCGTGTCACAGCGTACAAGGA |
| | | | GGGATATGGAGAAGGTTGCGTAACCATTCATGAGATCGAGAACAATACAGACGAACTGAAGTTT |
| | | | AGCAACTGTGTAGAAGAGGAAGTATATCCAAACAACACGGTAACGTGTAATGATTATACTGCGA |
| | | | CTCAAGAAGAATATGAGGGTACGTACACTTCTCGTAATCGAGGATATGACGGAGCCTATGAAAG |
| | | | CAATTCTTCTGTACCAGCTGATTATGCATCAGCCTATGAAGAAAAAGCATATACAGATGGACGA |
| | | | AGAGACAATCCTTGTGAATCTAACAGAGGATATGGGGATTACACACCACTACCAGCTGGCTATG |
| | | | TGACAAAAGAATTAGAGTACTTCCCAGAAACCGATAAGGTATGGATTGAGATCGGAGAAACGG |
| | | | AAGGAACATTCATCGTGGACAGCGTGGAATTACTTCTTATGGAGGAATAA |
| NRRL B- | Cry1Ab1 | 7 | ATGGATAACAATCCGAACATCAATGAATGCATTCCTTATAATTGTTTAAGTAACCCTGAAGTAGA |
| 67688 | | | AGTATTAGGTGGAGAAAGAATAGAAACTGGTTACACCCCAATCGATATTTCCTTGTCGCTAACG |
| | | | CAATTTCTTTTGAGTGAATTTGTTCCCGGTGCTGGATTTGTGTTAGGACTAGTTGATATAATATGG |
| | | | GGAATTTTTGGTCCCTCTCAATGGGACGCATTTCTTGTACAAATTGAACAGTTAATTAACCAAAG |
| | | | AATAGAAGAATTCGCTAGGAACCAAGCCATTTCTAGATTAGAAGGACTAAGCAATCTTTATCAA |
| | | | ATTTACGCAGAATCTTTTAGAGAGTGGGAAGCAGATCCTACTAATCCAGCATTAAGAGAAGAGA |
| | | | TGCGTATTCAATTCAATGACATGAACAGTGCCCTTACAACCGCTATTCCTCTTTTTGCAGTTCAA |
| | | | AATTATCAAGTTCCTCTTTTATCAGTATATGTTCAAGCTGCAAATTTACATTTATCAGTTTTGAGA |
| | | | GATGTTTCAGTGTTTGGACAAAGGTGGGGATTTGATGCCGCGACTATCAATAGTCGTTATAATGA |
| | | | TTTAACTAGGCTTATTGGCAACTATACAGATCATGCTGTACGCTGGTACAATACGGGATTAGAGC |
| | | | GTGTATGGGGACCGGATTCTAGAGATTGGATAAGATATAATCAATTTAGAAGAGAATTAACACT |
| | | | AACTGTATTAGATATCGTTTCTCTATTTCCGAACTATGATAGTAGAACGTATCCAATTCGAACAG |
| | | | TTTCCCAATTAACAAGAGAAATTTATACAAACCCAGTATTAGAAAATTTTGATGGTAGTTTTCGA |
| | | | GGCTCGGCTCAGGGCATAGAAGGAAGTATTAGGAGTCCACATTTGATGGATATACTTAACAGTA |
| | | | TAACCATCTATACGGATGCTCATAGAGGAGAATATTATTGGTCAGGGCATCAAATAATGGCTTCT |
| | | | CCTGTAGGGTTTTCGGGGCCAGAATTCACTTTTCCGCTATATGGAACTATGGGAAATGCAGCTCC |
| | | | ACAACAACGTATTGTTGCTCAACTAGGTCAGGGCGTGTATAGAACATTATCGTCCACTTTATATA |
| | | | GAAGACCTTTTAATATAGGGATAAATAATCAACAACTATCTGTTCTTGACGGGACAGAATTTGCT |
| | | | TATGGAACCTCCTCAAATTTGCCATCCGCTGTATACAGAAAAAGCGGAACGGTAGATTCGCTGG |
| | | | ATGAAATACCGCCACAGAATAACAACGTGCCACCTAGGCAAGGATTTAGTCATCGATTAAGCCA |
| | | | TGTTTCAATGTTTCGTTCAGGCTTTAGTAATAGTAGTGTAAGTATAATAAGAGCTCCTATGTTCTC |
| | | | TTGGATACATCGTAGTGCTGAATTTAATAATATAATTCCTTCATCACAAATTACACAAATACCTT |
| | | | TAACAAAATCTACTAATCTTGGCTCTGGAACTTCTGTCGTTAAAGGACCAGGATTTACAGGAGG |
| | | | AGATATTCTTCGAAGAACTTCACCTGGCCAGATTTCAACCTTAAGAGTAAATATTACTGCACCAT |
| | | | TATCACAAAGATATCGGGTAAGAATTCGCTACGCTTCTACCACAAATTTACAATTCCATACATCA |
| | | | ATTGACGGAAGACCTATTAATCAGGGGAATTTTTCAGCAACTATGAGTAGTGGGAGTAATTTAC |
| | | | AGTCCGGAAGCTTTAGGACTGTAGGTTTTACTACTCCGTTTAACTTTTCAAATGGATCAAGTGTA |
| | | | TTTACGTTAAGTGCTCATGTCTTCAATTCAGGCAATGAAGTTTATATAGATCGAATTGAATTTGTT |
| | | | CCGGCAGAAGTAACCTTTGAGGCAGAATATGATTTAGAAAGAGCACAAAAGGCGGTGAATGAG |
| | | | CTGTTTACTTCTTCCAATCAAATCGGGTTAAAAACAGATGTGACGGATTATCATATTGATCAAGT |
| | | | ATCCAATTTAGTTGAGTGTTTATCTGATGAATTTTGTCTGGATGAAAAAAAAGAATTGTCCGAGA |
| | | | AAGTCAAACATGCGAAGCGACTTAGTGATGAGCGGAATTTACTTCAAGATCCAAACTTTAGAGG |
| | | | GATCAATAGACAACTAGACCGTGGCTGGAGAGGAAGTACGGATATTACCATCCAAGGAGGCGA |
| | | | TGACGTATTCAAAGAGAATTACGTTACGCTATTGGGTACCTTTGATGAGTGCTATCCAACGTATT |
| | | | TATATCAAAAAATAGATGAGTCGAAATTAAAAGCCTATACCCGTTACCAATTAAGAGGGTATAT |
| | | | CGAAGATAGTCAAGACTTAGAAATCTATTTAATTCGCTACAATGCCAAACACGAAACAGTAAAT |
| | | | GTGCCAGGTACGGGTTCCTTATGGCCGCTTTCAGCCCCAAGTCCAATCGGAAAATGTGCCCATCA |
| | | | TTCCCATCATTTCTCCTTGGACATTGATGTTGGATGTACAGACTTAAATGAGGACTTAGGTGTAT |
| | | | GGGTGATATTCAAGATTAAGACGCAAGATGGCCATGCAAGACTAGGAAATCTAGAATTTCTCGA |
| | | | AGAGAAACCATTAGTAGGAGAAGCACTAGCTCGTGTGAAAAGAGCGGAGAAAAAATGGAGAG |
| | | | ACAAACGTGAAAAATTGGAATGGGAAACAAATATTGTTTATAAAGAGGCAAAAGAATCTGTAG |
| | | | ATGCTTTATTTGTAAACTCTCAATATGATAGATTACAAGCGGATACGAATATTGCCATGATTCAT |
| | | | GCGGCAGATAAACGCGTTCATAGCATTCGAGAAGCTTATCTGCCTGAGCTGTCTGTGATTCCGGG |
| | | | TGTCAATGCGGCTATTTTTGAAGAATTAGAAGGGCGTATTTTCACTGCATTCTCCCTATATGATG |
| | | | CGAGAAATGTCATTAAAAATGGTGATTTTAATAATGGCTTATCCTGCTGGAACGTGAAAGGGCA |
| | | | TGTAGATGTAGAAGAACAAAACAACCAACGTTCGGTCCTTGTTGTTCCGGAATGGGAAGCAGAA |
| | | | GTGTCACAAGAAGTTCGTGTCTGTCCGGGTCGTGGCTATATCCTTCGTGTCACAGCGTACAAGGA |
| | | | GGGATATGGAGAAGGTTGCGTAACCATTCATGAGATCGAGAACAATACAGACGAACTGAAGTTT |
| | | | AGCAACTGTGTAGAAGAGGAAGTATATCCAAACAACACGGTAACGTGTAATGATTATACTGCGA |
| | | | CTCAAGAAGAATATGAGGGTACGTACACTTCTCGTAATCGAGGATATGACGGAGCCTATGAAAG |
| | | | CAATTCTTCTGTACCAGCTGATTATGCATCAGCCTATGAAGAAAAAGCATATACAGATGGACGA |
| | | | AGAGACAATCCTTGTGAATCTAACAGAGGATATGGGGATTACACACCACTACCAGCTGGCTATG |
| | | | TGACAAAAGAATTAGAGTACTTCCCAGAAACCGATAAGGTATGGATTGAGATCGGAGAAACGG |
| | | | AAGGAACATTCATCGTGGACAGCGTGGAATTACTTCTTATGGAGGAATAA |

TABLE 4
Amino acid sequences of insecticidal toxins in which Bacillus
thuringiensis strains NRRL B-67685, NRRL B-67687, and NRRL B-67688
share 100% sequence identity.

| | SEQ ID | |
|---|---|---|
| Protein | NO: | Sequence |
| Vip3Aa11 | 8 | MNKNNTKLSTRALPSFIDYFNGIYGFATGIKDIMNMIFKTDTGGDLTLDEILKNQQLLNDISGKLDGVNGSLNDLI |
| or VIP3Aa7 | | AQGNLNTELSKEILKIANEQNQVLNDVNNKLDAINTMLRVYLPKITSMLSDVMKQNYALSLQIEYLSKQLQEISD |
| | | KLDIINVNVLINSTLTEITPAYQRIKYVNEKFEELTFATETSSKVKKDGSPADILDELTELTELAKSVTKNDVDGFEF |
| | | YLNTFHDVMVGNNLFGRSALKTASELITKENVKTSGSEVGNVYNFLIVLTALQAKAFLTLTTCRKLLGLADIDYT |
| | | SIMNEHLNKEKEEFRVNILPTLSNTFSNPNYAKVKGSDEDAKMIVEAKPGHALIGFEISNDSITVLKVYEAKLKQN |
| | | YQVDKDSLSEVIYGDMDKLLCPDQSEQIYYTNNIVFPNEYVITKIDFTKKMKTLRYEVTANFYDSSTGEIDLNKK |
| | | KVESSEAEYRTLSANDDGVYMPLGVISETFLTPINGFGLQADENSRLITLTCKSYLRELLLATDLSNKETKLIVPPS |
| | | GFISNIVENGSIEEDNLEPWKANNKNAYVDHTGGVNGTKALYVHKDGGISQFIGDKLKPKTEYVIQYTVKGKPSI |
| | | HLKDENTGYIHYEDTNNNLEDYQTINKRFTTGTDLKGVYLILKSQNGDEAWGDNFIILEISPSEKLLSPELINTNN |
| | | WTSTGSTNISGNTLTLYQGGRGILKQNLQLDSFSTYRVYFSVSGDANVRIRNSREVLFEKRYMSGAKDVSEMFTT |
| | | KFEKDNFYIELSQGNNLYGGPIVHFYDVSIK |
| Cry1Ia2 | 9 | MKLKNQDKHQSFSSNAKVDKISTDSLKNETDIELQNINHEDCLKMSEYENVEPFVSASTIQTGIGIAGKILGTLGV |
| | | PFAGQVASLYSFILGELWPKGKNQWEIFMEHVEEIINQKISTYARNKALTDLKGLGDALAVYHDSLESWVGNRN |
| | | NTRARSVVKSQYIALELMFVQKLPSFAVSGEEVPLLPIYAQAANLHLLLLRDASIFGKEWGLSSSEISTFYNRQVE |
| | | RAGDYSDHCVKWYSTGLNNLRGTNAESWVRYNQFRRDMTLMVLDLVALFPSYDTQMYPIKTTAQLTREVYTD |
| | | AIGTVHPHPSFTSTTWYNNNAPSFSAIEAAVVRNPHLLDFLEQVTIYSLLSRWSNTQYMNMWGGHKLEFRTIGGT |
| | | LNISTQGSTNTSINPVTLPFTSRDVYRTESLAGLNLFLTQPVNGVPRVDFHWKFVTHPIASDNFYYPGYAGIGTQL |
| | | QDSENELPPEATGQPNYESYSHRLSHIGLISASHVKALVYSWTHRSADRTNTIEPNSITQIPLVKAFNLSSGAAVVR |
| | | GPGFTGGDILRRTNTGTFGDIRVNINPPFAQRYRVRIRYASTTDLQFHTSINGKAINQGNFSATMNRGEDLDYKTF |
| | | RTVGFTTPFSFLDVQSTFTIGAWNFSSGNEVYIDRIEFVPVEVTYEAEYDFEKAQEKVTALFTSTNPRGLKTDVKD |
| | | YHIDQVSNLVESLSDEFYLDEKRELFEIVKYAKQLHIERNM |
| Cry2Ab1 | 10 | MNSVLNSGRTTICDAYNVAAHDPFSFQHKSLDTVQKEWTEWKKNNHSLYLDPIVGTVASFLLKKVGSLVGKRIL |
| | | SELRNLIFPSGSTNLMQDILRETEKFLNQRLNTDTLARVNAELTGLQANVEEFNRQVDNFLNPNRNAVPLSITSSV |
| | | NTMQQLFLNRLPQFQMQGYQLLLLPLFAQAANLHLSFIRDVILNADEWGISAATLRTYRDYLKNYTRDYSNYCI |
| | | NTYQSAFKGLNTRLHDMLEFRTYMFLNVFEYVSIWSLFKYQSLLVSSGANLYASGSGPQQTQSFTSQDWPFLYS |
| | | LFQVNSNYVLNGFSGARLSNTFPNIVGLPGSTTTHALLAARVNYSGGISSGDIGASPFNQNFNCSTFLPPLLTPFVR |
| | | SWLDSGSDREGVATVTNWQTESFETTLGLRSGAFTARGNSNYFPDYFIRNISGVPLVVRNEDLRRPLHYNEIRNIA |
| | | SPSGTPGGARAYMVSVHNRKNNIHAVHENGSMIHLAPNDYTGFTISPIHATQVNNQTRTFISEKFGNQGDSLRIE |
| | | QNNTTARYTLRGNGNSYNLYLRVSSIGNSTIRVTINGRVYTATNVNTTTNNDGVNDNGARFSDINIGNVVASSNS |
| | | DVPLDINVTLNSGTQFDLMNIMLVPTNISPLY |

TABLE 5
Amino acid sequences of insecticidal toxins in which Bacillus
thuringiensis strains NRRL B-67685, NRRL B-67687, and NRRL B-67688
share at least 99.9% sequence identity

| | Amino | | |
|---|---|---|---|
| | Acid | SEQ ID | |
| Strain(s) | Sequence | NO: | Sequence |
| NRRL B- | Cry1Aa8 | 11 | MDNNPNINECIPYNCLSNPEVEVLGGERIETGYTPIDISLSLTQFLLSEFVPGAGFVLGLVDIIWGIFGPS |
| 67687 & | or | | QWDAFLVQIEQLINQRIEEFARNQAISRLEGLSNLYQIYAESFREWEADPTNPALREEMRIQFNDMNS |
| NRRL B- | Cry1Aa11 | | ALTTAIPLLAVQNYQVPLLSVYVQAANLHLSVLRDVSVFGQRWGFDAATINSRYNDLTRLIGNYTDY |
| 67688 | | | AVRWYNTGLERVWGPDSRDWVRYNQFRRELTLTVLDIVALFSNYDSRRYPIRTVSQLTREIYTNPVL |
| | | | ENFDGSFRGMAQRIEQNIRQPHLMDILNSITIYTDVHRGFNYWSGHQITASPVGFSGPEFAFPLFGNAG |
| | | | NAAPPVLVSLTGLGIFRTLSSPLYRRIILGSGPNNQELFVLDGTEFSFASLTTNLPSTIYRQRGTVDSLD |
| | | | VIPPQDNSVPPRAGFSHRLSHVTMLSQAAGAVYTLRAPTFSWQHRSAEFNNIIPSSQITQIPLTKSTNLG |
| | | | SGTSVVKGPGFTGGDILRRTSPGQISTLRVNITAPLSQRYRVRIRYASTTNLQFHTSIDGRPINQGNFSA |
| | | | TMSSGSNLQSGSFRTVGFTTPFNFSNGSSVFTLSAHVFNSGNEVYIDRIEFVPAEVTPEAEYDLERAQK |
| | | | AVNELFTSSNQIGLKTDVTDYHIDQVSNLVECLSDEFCLDEKQELSEKVKHAKRLSDERNLLQDPNFR |
| | | | GINRQLDRGWRGSTDITIQGGDDVFKENYVTLLGTFDECYPTYLYQKIDESKLKAYTRYQLRGYIEDS |
| | | | QDLEIYLIRYNAKHETVNVPGTGSLWPLSAQSPIGKCGEPNRCAPHLEWNPDLDCSCRDGEKCAHHS |
| | | | HHFSLDIDVGCTDLNEDLGVWVIFKIKTQDGHARLGNLEFLEEKPLVGEALARVKRAEKKWRDKRE |
| | | | KLEWETNIVYKEAKESVDALFVNSQYDQLQADTNIAMIHAADKRVHSIREAYLPELSVIPGVNAAIIE |
| | | | ELEGRIFTAFSLYDARNVIKNGDFNNGLSCWNVKGHVDVEEQNNQRSVLVVPEWEAEVSQEVRVCP |
| | | | GRGYILRVTAYKEGYGEGCVTIHEIENNTDELKFSNCVEEEIYPNNTVTCNDYTVNQEEYGGAYTSR |
| | | | NRGYNEAPSVPADYASVYEEKSYTDGRRENPCEFNRGYRDYTPLPVGYVTKELEYFPETDKVWIEIG |
| | | | ETEGTFIVDSVELLLMEE |
| NRRL B- | Cry1Aa3 | 12 | MDNNPNINECIPYNCLSNPEVEVLGGERIETGYTPIDISLSLTQFLLSEFVPGAGFVLGLVDIIWGIFGPS |
| 67685 | | | QWDAFLVQIEQLINQRIEEFARNQAISRLEGLSNLYQIYAESFREWEADPTNPALREEMRIQFNDMNS |
| | | | ALTTAIPLFAVQNYQVPLLSVYVQAANLHLSVLRDVSVFGQRWGFDAATINSRYNDLTRLIGNYTDY |
| | | | AVRWYNTGLERVWGPDSRDWVRYNQFRRELTLTVLDIVALFSNYDSRRYPIRTVSQLTREIYTNPVL |
| | | | ENFDGSFRGMAQRIEQNIRQPHLMDILNSITIYTDVHRGFNYWSGHQITASPVGFSGPEFAFPLFGNAG |
| | | | NAAPPVLVSLTGLGIFRTLSSPLYRRIILGSGPNNQELFVLDGTEFSFASLTTNLPSTIYRQRGTVDSLD |
| | | | VIPPQDNSVPPRAGFSHRLSHVTMLSQAAGAVYTLRAPTFSWQHRSAEFNNIIPSSQITQIPLTKSTNLG |
| | | | SGTSVVKGPGFTGGDILRRTSPGQISTLRVNITAPLSQRYRVRIRYASTTNLQFHTSIDGRPINQGNFSA |
| | | | TMSSGSNLQSGSFRTVGFTTPFNFSNGSSVFTLSAHVFNSGNEVYIDRIEFVPAEVTPEAEYDLERAQK |
| | | | AVNELFTSSNQIGLKTDVTDYHIDQVSNLVECLSDEFCLDEKQELSEKVKHAKRLSDERNLLQDPNFR |
| | | | GINRQLDRGWRGSTDITIQGGDDVFKENYVTLLGTFDECYPTYLYQKIDESKLKAYTRYQLRGYIEDS |
| | | | QDLEIYLIRYNAKHETVNVPGTGSLWPLSAQSPIGKCGEPNRCAPHLEWNPDLDCSCRDGEKCAHHS |
| | | | HHFSLDIDVGCTDLNEDLGVWVIFKIKTQDGHARLGNLEFLEEKPLVGEALARVKRAEKKWRDKRE |
| | | | KLEWETNIVYKEAKESVDALFVNSQYDQLQADTNIAMIHAADKRVHSIREAYLPELSVIPGVNAAIIE |
| | | | ELEGRIFTAFSLYDARNVIKNGDFNNGLSCWNVKGHVDVEEQNNQRSVLVVPEWEAEVSQEVRVCP |
| | | | GRGYILRVTAYKEGYGEGCVTIHEIENNTDELKFSNCVEEEIYPNNTVTCNDYTVNQEEYGGAYTSR |
| | | | NRGYNEAPSVPADYASVYEEKSYTDGRRENPCEFNRGYRDYTPLPVGYVTKELEYFPETDKVWIEIG |
| | | | ETEGTFIVDSVELLLMEE |
| NRRL B- | Cry1Ab1 | 13 | MDNNPNINECIPYNCLSNPEVEVLGGERIETGYTPIDISLSLTQFLLSEFVPGAGFVLGLVDIIWGIFGPS |
| 67685 & | | | QWDAFLVQIEQLINQRIEEFARNQAISRLEGLSNLYQIYAESFREWEADPTNPALREEMRIQFNDMNS |
| NRRL B- | | | ALTTAIPLFAVQNYQVPLLSVYVQAANLHLSVLRDVSVFGQRWGFDAATINSRYNDLTRLIGNYTDH |
| 67687 | | | AVRWYNTGLERVWGPDSRDWIRYNQFRRELTLTVLDIVSLFPNYDSRTYPIRTVSQLTREIYTNPVLE |
| | | | NFDGSFRGSAQGIEGSIRSPHLMDILNSITIYTDAHRGEYYWSGHQIMASPVGFSGPEFTFPLYGTMGN |
| | | | AAPQQRIVAQLGQGVYRTLSSTLYRRPFNIGINNQQLSVLDGTEFAYGTSSNLPSAVYRKSGTVDSLD |
| | | | EIPPQNNNVPPRQGFSHRLSHVSMFRSGFSNSSVSIIRAPMFSWIHRSAEFNNIIPSSQITQIPLTKSTNLG |
| | | | SGTSVVKGPGFTGGDILRRTSPGQISTLRVNITAPLSQRYRVRIRYASTTNLQFHTSIDGRPINQGNFSA |
| | | | TMSSGSNLQSGSFRTVGFTTPFNFSNGSSVFTLSAHVFNSGNEVYIDRIEFVPAEVTPEAEYDLERAQK |
| | | | AVNELFTSSNQIGLKTDVTDYHIDQVSNLVECLSDEFCLDEKKELSEKVKHAKRLSDERNLLQDPNFR |
| | | | GINRQLDRGWRGSTDITIQGGDDVFKENYVTLLGTFDECYPTYLYQKIDESKLKAYTRYQLRGYIEDS |
| | | | QDLEIYLIRYNAKHETVNVPGTGSLWPLSAPSPIGKCAHHSHHFSLDIDVGCTDLNEDLGVWVIFKIK |
| | | | TQDGHARLGNLEFLEEKPLVGEALARVKRAEKKWRDKREKLEWETNIVYKEAKESVDALFVNSQY |
| | | | DRLQADTNIAMIHAADKRVHSIREAYLPELSVIPGVNAAIPEELEGRIFTAFSLYDARNVIKNGDFNNG |
| | | | LSCWNVKGHVDVEEQNNHRSVLVVPEWEAEVSQEVRVCPGRGYILRVTAYKEGYGEGCVTIHEIEN |
| | | | NTDELKFSNCVEEEVYPNNTVTCNDYTATQEEYEGTYTSRNRGYDGAYESNSSVPADYASAYEEKA |
| | | | YTDGRRDNPCESNRGYGDYTPLPAGYVTKELEYFPETDKVWIEIGETEGTFIVDSVELLLMEE |
| NRRL B- | Cry1Ab1 | 14 | MDNNPNINECIPYNCLSNPEVEVLGGERIETGYTPIDISLSLTQFLLSEFVPGAGFVLGLVDIIWGIFGPS |
| 67688 | | | QWDAFLVQIEQLINQRIEEFARNQAISRLEGLSNLYQIYAESFREWEADPTNPALREEMRIQFNDMNS |
| | | | ALTTAIPLFAVQNYQVPLLSVYVQAANLHLSVLRDVSVFGQRWGFDAATINSRYNDLTRLIGNYTDH |
| | | | AVRWYNTGLERVWGPDSRDWIRYNQFRRELTLTVLDIVSLFPNYDSRTYPIRTVSQLTREIYTNPVLE |
| | | | NFDGSFRGSAQGIEGSIRSPHLMDILNSITIYTDAHRGEYYWSGHQIMASPVGFSGPEFTFPLYGTMGN |
| | | | AAPQQRIVAQLGQGVYRTLSSTLYRRPFNIGINNQQLSVLDGTEFAYGTSSNLPSAVYRKSGTVDSLD |
| | | | EIPPQNNNVPPRQGFSHRLSHVSMFRSGFSNSSVSIIRAPMFSWIHRSAEFNNIIPSSQITQIPLTKSTNLG |
| | | | SGTSVVKGPGFTGGDILRRTSPGQISTLRVNITAPLSQRYRVRIRYASTTNLQFHTSIDGRPINQGNFSA |
| | | | TMSSGSNLQSGSFRTVGFTTPFNFSNGSSVFTLSAHVFNSGNEVYIDRIEFVPAEVTPEAEYDLERAQK |
| | | | AVNELFTSSNQIGLKTDVTDYHIDQVSNLVECLSDEFCLDEKKELSEKVKHAKRLSDERNLLQDPNFR |
| | | | GINRQLDRGWRGSTDITIQGGDDVFKENYVTLLGTFDECYPTYLYQKIDESKLKAYTRYQLRGYIEDS |
| | | | QDLEIYLIRYNAKHETVNVPGTGSLWPLSAPSPIGKCAHHSHHFSLDIDVGCTDLNEDLGVWVIFKIK |
| | | | TQDGHARLGNLEFLEEKPLVGEALARVKRAEKKWRDKREKLEWETNIVYKEAKESVDALFVNSQY |
| | | | DRLQADTNIAMIHAADKRVHSIREAYLPELSVIPGVNAAIPEELEGRIFTAFSLYDARNVIKNGDFNNG |
| | | | LSCWNVKGHVDVEEQNNQRSVLVVPEWEAEVSQEVRVCPGRGYILRVTAYKEGYGEGCVTIHEIEN |
| | | | NTDELKFSNCVEEEVYPNNTVTCNDYTATQEEYEGTYTSRNRGYDGAYESNSSVPADYASAYEEKA |
| | | | YTDGRRDNPCESNRGYGDYTPLPAGYVTKELEYFPETDKVWIEIGETEGTFIVDSVELLLMEE |

The genome sequence of Bacillus thuringiensis strain NRRL B-67685 was further analyzed. This analysis revealed that the strain has insecticidal toxin genes for Cry1Ca and Cry1Da, the nucleotide and amino acid sequences of which are provided as SEQ ID NO: 15 (amino acid sequence for Cry1Ca8), SEQ ID NO: 16 (nucleic acid sequence for Cry1Ca8), SEQ ID NO: 17 (amino acid sequence for Cry1Da1) and SEQ ID NO: 18 (nucleic acid sequence for Cry1Da1). Proteomic analyses indicated that NRRL B-67685 expresses Cry1Aa3, Cry1Ab1, Cry1Ca8, Cry1Da1 and Vip3Aa7.

### Example 4. Synergistic Insecticidal Activity of Zwittermicin A and Vip3A with Spodoptera exigua H\ddot{υ}bner

Zwittermicin A was partially purified from a strain derived from Bacillus thuringiensis strain NRRL B-67688. After growing the strain in a soy-based medium, the whole broth was centrifuged and the supernatant removed. The supernatant was passed through a 3 kDa filter to separate the zwittermicin A from larger molecules including any Cry toxins. The Vip3Aa11 protein was produced in the expression strain of Escherichia coli BL21 and applied to insect larvae as the E. coli whole broth culture (WB) at concentrations of 0.1%, 1%, and 10%. Treatment groups included Vip3Aa11 only (“no Zwa”); Vip3Aa11 with zwittermicin A (“1×Zwa); and Vip3Aa11 with concentrated zwittermicin A (“2×Zwa”) containing twice as much zwittermicin. Concentrations of zwittermicin A and Vip3Aa11 were adjusted by adding deionized water to prepare the appropriate dilutions. Control treatments included zwittermicin A (“1×Zwa”) alone, concentrated zwittermicin A alone (“2×Zwa”), untreated control, and a positive control containing 1000 ppm of a commercially available biological control agent active against Lepidoptera.

To evaluate the insecticidal activity of each treatment, second instars of Spodoptera exigua H\ddot{υ}bner (beet armyworm) were grown on 48-well plates containing an agar substrate similar to that described in Marrone et al., (1985), “Improvements in Laboratory Rearing of the Southern Corn Rootworm, Diabrotica undecimpuncta howardi Barber (Coleoptera: Chrysomelidae), on an Artificial Diet and Corn,” J. Econ. Entomol. 78: 290-293. Each treatment was applied to the agar substrate and a second instar was then placed in the well. After several days, insect development and survival were evaluated. Insect development scores were rated according to the following scale: 1=severely stunted; 2=highly stunted, minimal growth; 3=slightly smaller than untreated control; 4=same size as untreated control.

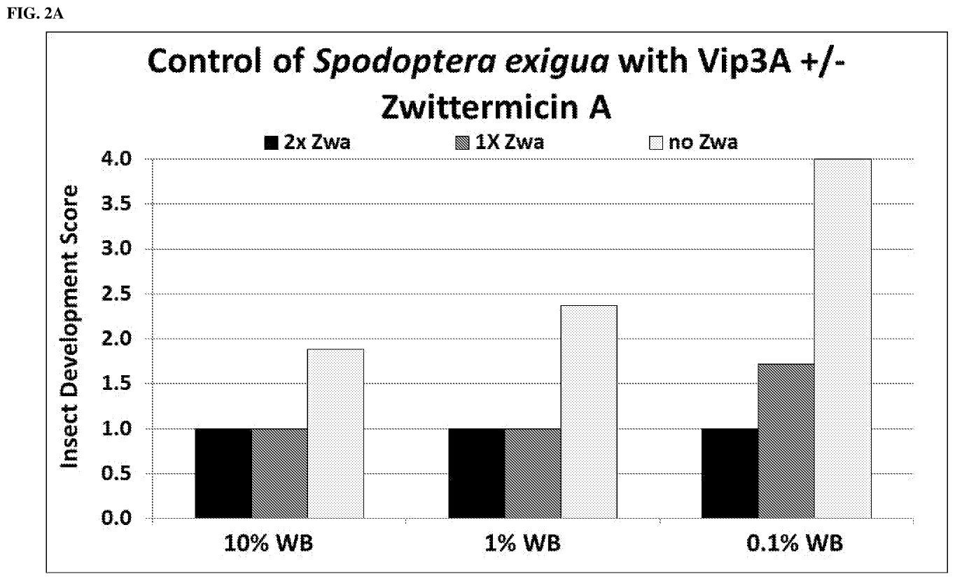

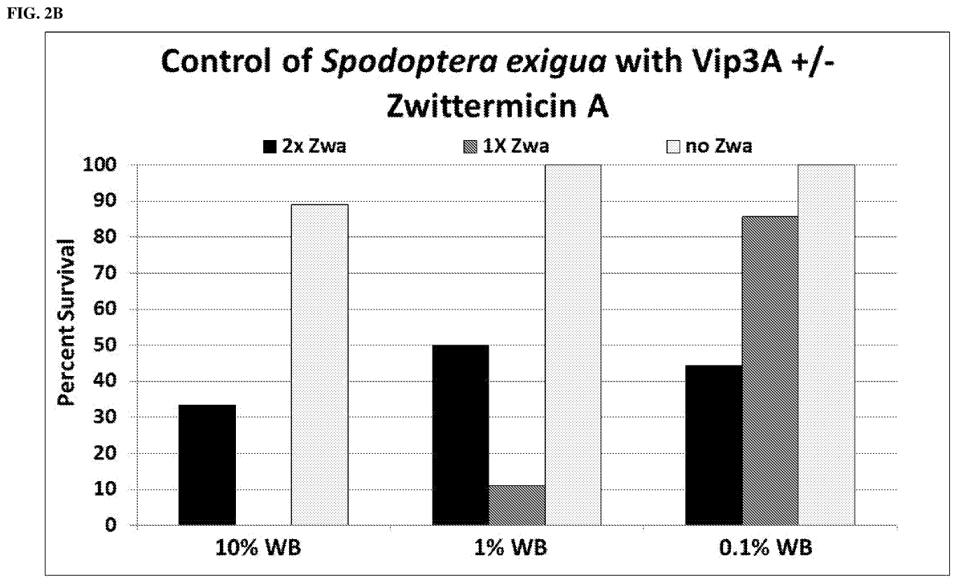

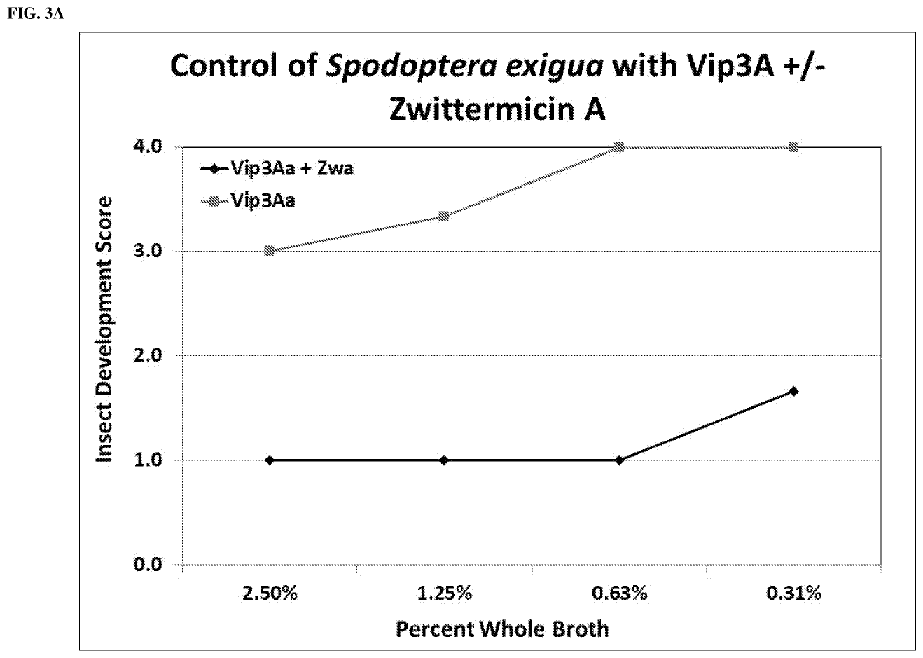

Application of zwittermicin alone (i.e., 1×Zwa or 2×Zwa) to the plates had no significant effect on insect growth or survival. Second instars treated with Vip3Aa11 alone experienced a stunting of growth at application rates of 1% WB and 10% WB and a slight decrease in survival at the application rate of 10% WB. Surprisingly, addition of zwittermicin A to the Vip3Aa11 treatments increased both developmental delay and mortality in every instance including at the lowest Vip3Aa11 application rate of 0.1% WB indicating a synergistic effect arising from the combination (see FIGS. 2A and 2B).

### Example 5. Synergistic Insecticidal Activity of Zwittermicin A and Vip3A with Other Lepidoptera Species

The experiment conducted in Example 4 was repeated with other Lepidoptera species and at differing concentrations E. coli whole broth containing the heterologously expressed Vip3Aa11. Only the more dilute concentration of zwittermicin A (i.e., “1×Zwa”) was evaluated in the treatments. Second instars of each Lepidoptera species were used, and the insect development scores reported are the average of three replicates. Insect development was scored as described in Example 3. For assays with Spodoptera exigua Hübner (beet armyworm) and Trichoplusia ni (cabbage looper), the Vip3Aa11 whole broth was applied to the plates at concentrations of 0.31%, 0.63%, 1.25%, and 2.50%. Assays with Plutella xylostella (Linnaeus) (diamondback moth) evaluated Vip3Aa11 whole broth at concentrations of 6.25%, 12.5%, 25%, and 50%. A strain of Plutella xylostella (Linnaeus) (diamondback moth) resistant to treatment with DIPEL® (Bacillus thuringiensis subsp. kurstaki strain HD1) was included for evaluation with the treatments.

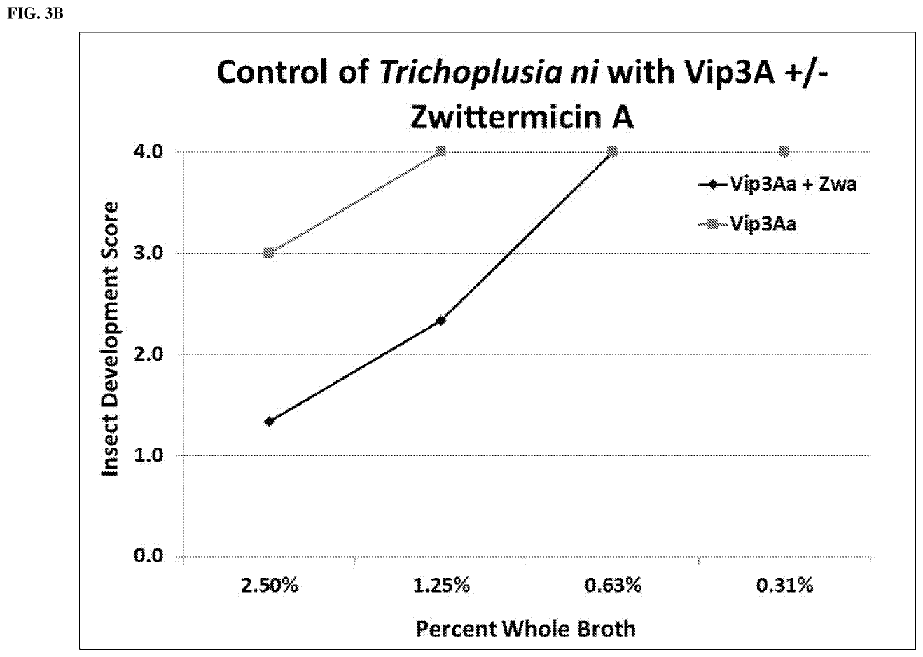

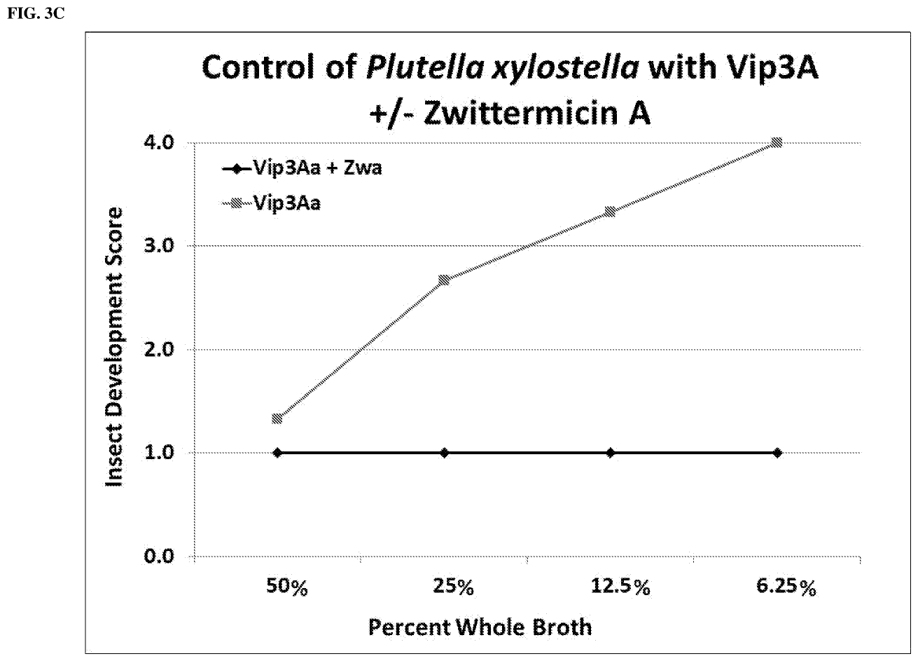

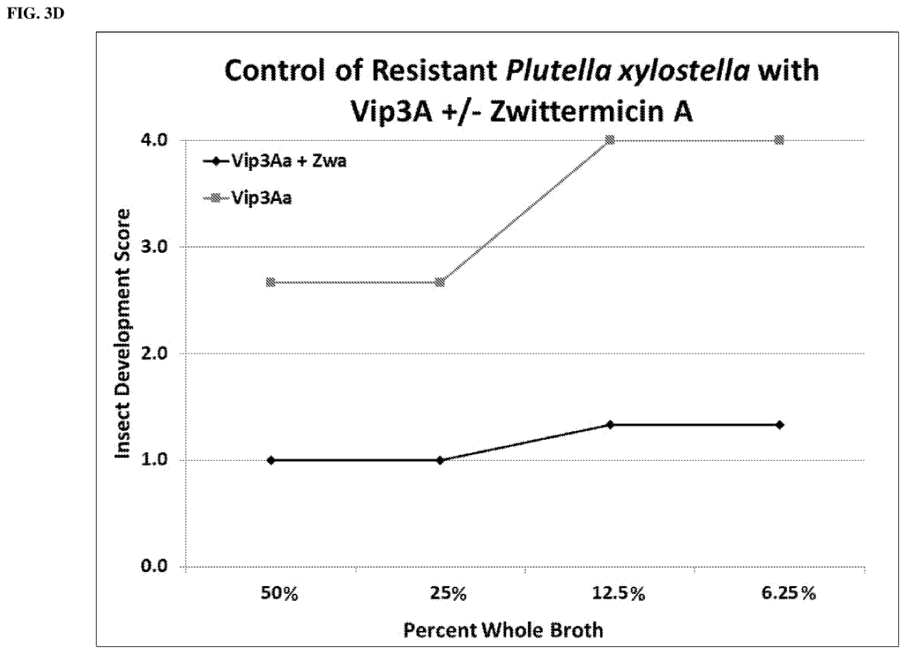

A control treatment containing the E. coli whole broth without induced expression of Vip3Aa11 showed no insecticidal activity against any of the Lepidoptera species. In each species tested, Vip3Aa11 alone had little effect on insect development. Strikingly, addition of zwittermicin A to the Vip3Aa11 treatments enhanced developmental delay with every species tested including the resistant strain of Plutella xylostella (Linnaeus) (diamondback moth) (see FIGS. 3A, 3B, 3C, and 3D).

### Example 6. Synergistic Insecticidal Activity Against Spodoptera exigua H\ddot{υ}bner of Zwittermicin A with Cry1Ab1, Cry1Ia2, Cry2Ab1, Cry1Ca1 and Cry1Da1

The experiment conducted in Example 3 was repeated with Cry1Ab1, Cry1Ia2, or Cry2Ab1 expressed in Escherichia coli BL21 and applied to insect larvae as the E. coli whole broth culture (WB) at concentrations of 0.2%, 4%, 10%, and 50%. The insect mortality score is based on the following scale: 4=0-25% mortality, 3=26-50% mortality, 2=51-79% mortality, 1=80-100% mortality. All percentages are adjusted according to any mortality observed with untreated control Spodoptera exigua H\ddot{υ}bner (beet armyworm) larvae. Only the more dilute concentration of zwittermicin A (i.e., “1×Zwa”) was evaluated in the treatments, and this concentration of zwittermicin A generally had no observable effect on insect mortality. All reported mortality scores are the average of three replicate measurements.

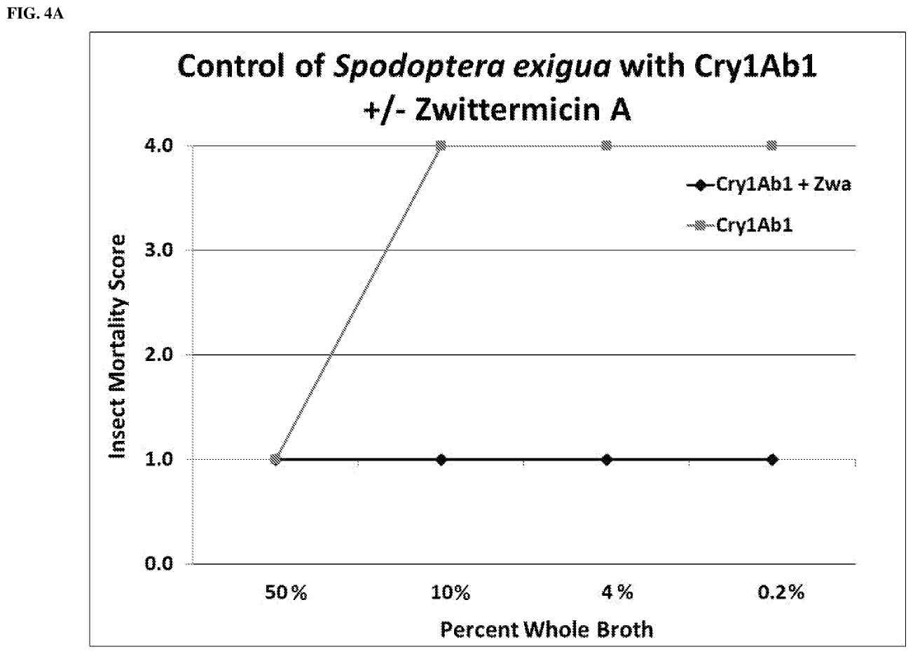

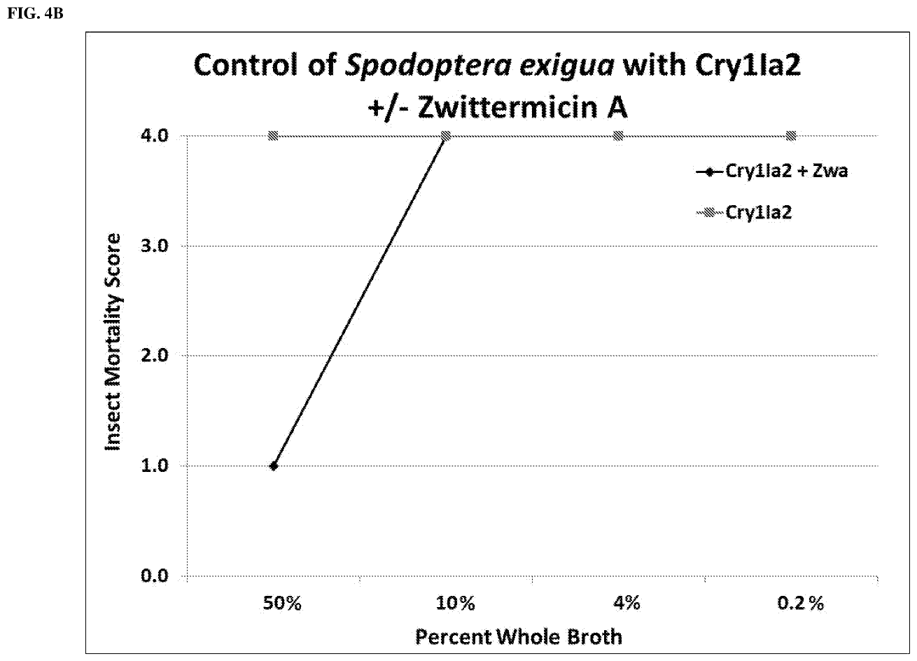

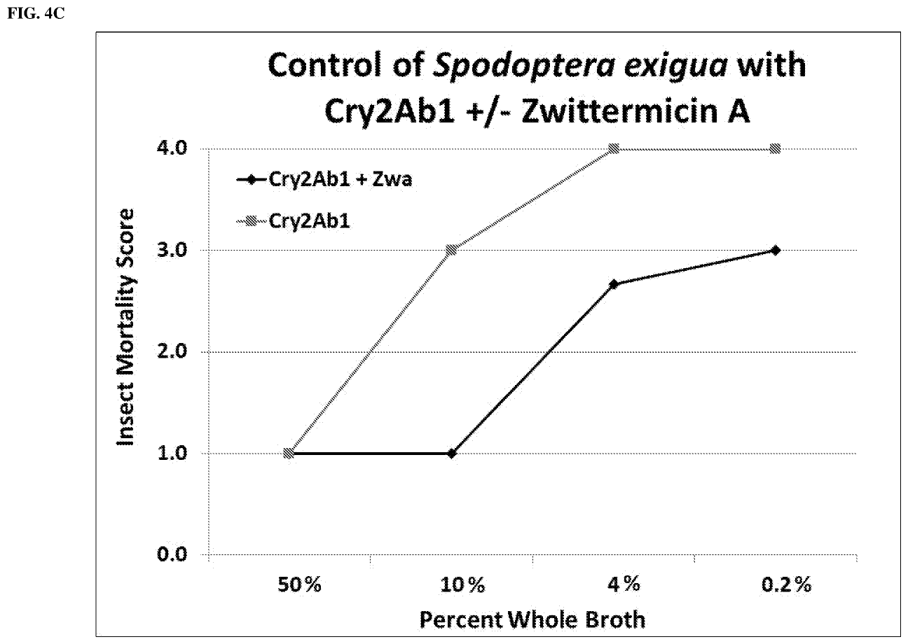

Cry1Ab1 and Cry2Ab1 applied alone demonstrated insect mortality at the higher application rates whereas Cry1Ia2 applied alone had little observable effect on insect mortality. When each of these Cry toxins was applied in combination with zwittermicin A, a significant increase in insect mortality was observed indicating a synergistic effect (see FIGS. 4A, 4B, and 4C).

The experiment in Example 3 was also repeated with Cry1Ca1 and Cry1Da1, which were expressed in Escherichia coli BL21 and applied to insect larvae as the E. coli whole broth culture (WB) at various concentrations. Instead of insect mortality rates, LC50, which in this case is the percentage of whole broth needed to cause 50% mortality, without zwittermicin or with various concentrations of zwittermicin, was determined. Results are shown in Table 6, below. The concentrations of zwittermicin used in this experiment generally had no observable effect on insect mortality.

TABLE 6

| | No | 5x | 2.5x | 1.25x | 0.625x | 0.3125x | 0.15x |
|---|---|---|---|---|---|---|---|
| ~LC50 | Zwa | Zwa | Zwa | Zwa | Zwa | Zwa | Zwa |
| Cry1Ca1 | 5-10 | <0.3 | <0.3 | <0.3 | <0.3 | <0.3 | <0.3 |
| Cry1Da1 | >10 | 0.3- | 0.6- | 0.3- | 0.6- | 0.6- | 1.2- |
| | | 0.6 | 1.2 | 0.6 | 1.2 | 1.2 | 2.5 |

### Example 7. Comparison of Insecticidal Activity of Bacillus thuringiensis Strains NRRL B-67685, NRRL B-67687, and NRRL B-67688 with Other Bacillus thuringiensis Strains

Whole broth cultures were produced in a soy-based medium with Bacillus thuringiensis strains NRRL B-67685, NRRL B-67687, and NRRL B-67688 along with several commercial strains and seven additional Bacillus thuringiensis strains. The commercial strains were DELIVER® (Bacillus thuringiensis subspecies kurstaki strain SA-12); DIPEL® (Bacillus thuringiensis subsp. kurstaki strain HD1); JAVELIN® (Bacillus thuringiensis subspecies kurstaki strain SA-11); AGREE® (Bacillus thuringiensis subspecies aizawai strain GC-91); XENTARI® (Bacillus thuringiensis subsp. aizawai strain ABTS-1857); and CRYMAX® (Bacillus thuringiensis subsp. kurstaki strain EG7841).

Insecticidal assays with Spodoptera exigua H\ddot{υ}bner (beet armyworm) were performed according to the protocol described in Example 4. Application rates of the Bacillus thuringiensis whole broths began at 50% and continued with 1:1 dilutions to lower concentrations. Insect mortality was determined several days after the larvae were exposed to each treatment. A culture media blank was included as a control. Mortality was reported as the average LD₅₀ (i.e., the average application rate required to kill half of the treated larvae). An LD₅₀ reported as 50% whole broth (e.g., the LD₅₀ for the media blank) indicates that the median lethal dose was greater than the highest concentration tested.

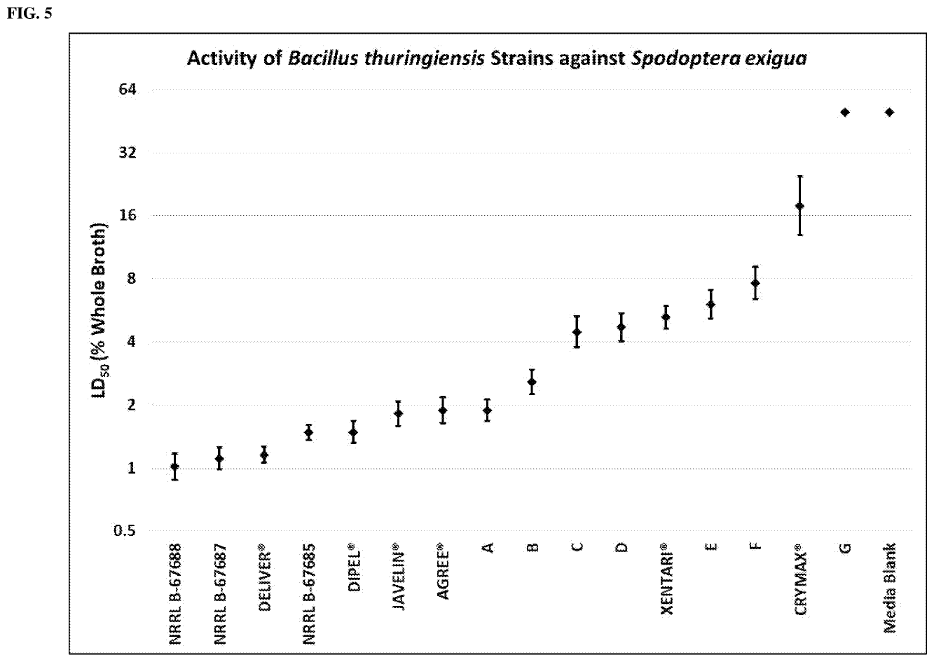

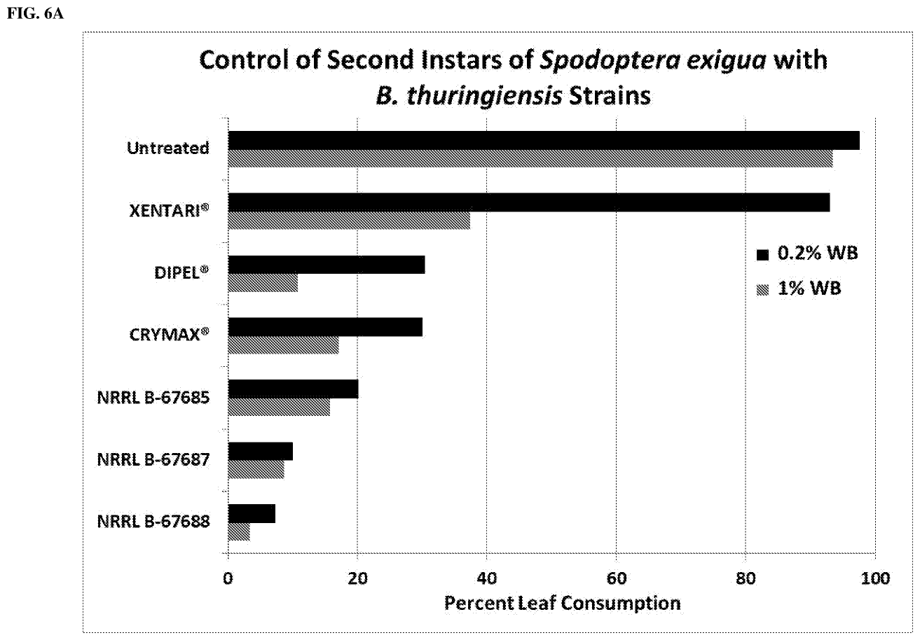

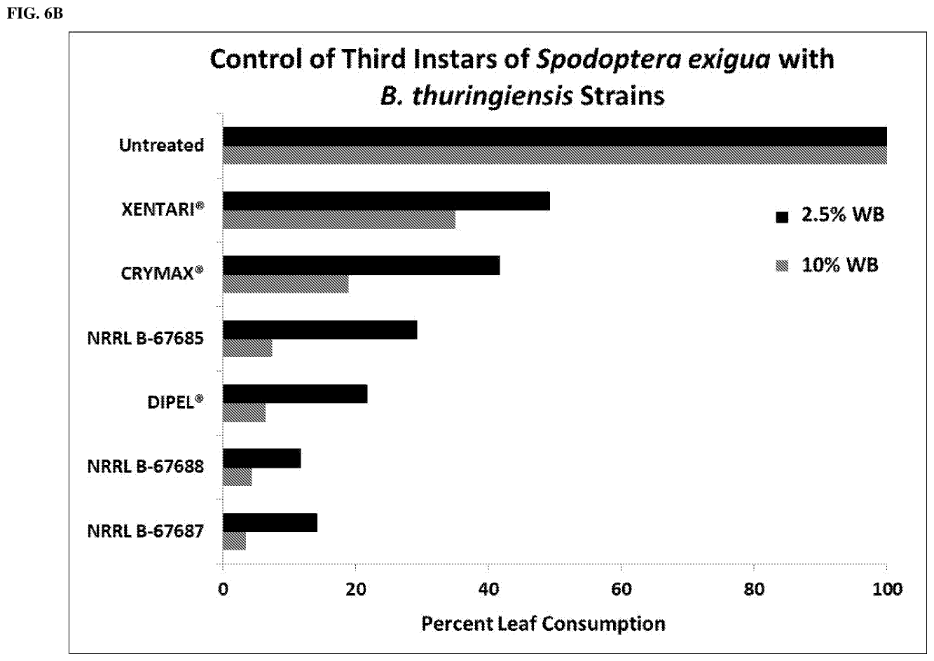

Bacillus thuringiensis strains NRRL B-67685, NRRL B-67687, and NRRL B-67688 produced superior insecticidal activity compared to the majority of the strains evaluated with control of Spodoptera exigua H\ddot{υ}bner (beet armyworm) similar to or exceeding that of DELIVER® (Bacillus thuringiensis subspecies kurstaki strain SA-12) (see FIG. 5).

Without wishing to be bound to a theory, the relatively high levels of zwittermicin A together with the unique profile of insecticidal toxins shared by Bacillus thuringiensis strains NRRL B-67685, NRRL B-67687, and NRRL B-67688 may be responsible for the superior insecticidal activity observed with these strains.

### Example 8. Activity of Strains Against Second and Third Instars of Spodoptera exigua

A similar experimental setup to that used in Example 4 was used to evaluate leaf consumption by second and third instars of Spodoptera exigua Hübner (beet armyworm) except that instead of an agar medium the wells contained leaf discs. Treatments also differed in that whole broth cultures of each of the following strains grown in a soy-based medium were applied to the leaf discs: XENTARI® (Bacillus thuringiensis subsp. aizawai strain ABTS-1857); DIPEL® (Bacillus thuringiensis subsp. kurstaki strain HD1); CRYMAX® (Bacillus thuringiensis subsp. kurstaki strain EG7841); and Bacillus thuringiensis strains NRRL B-67685, NRRL B-67687, and NRRL B-67688. For assays with second instars, whole broth cultures (WB) were applied to leaf discs at concentrations of 0.2% and 1%, and for those with third instars WB concentrations were 2.5% and 10%. Untreated control leaf discs were included for purposes of comparison. After treatment of the leaf discs, second or third instars were places in the wells. Several days later, the percent of the leaf consumed was recorded. Leaf consumption measurements reported are the averages of six replicates.

In the assays with second instars, Bacillus thuringiensis strains NRRL B-67685, NRRL B-67687, and NRRL B-67688 outperformed each of the commercial strains with Bacillus thuringiensis strain NRRL B-67688 producing the largest decrease in leaf consumption. With the larger third instars, each of the three strains outperformed the commercial strains present in XENTARI® and CRYMAX® and performed on a level similar to or slightly better than observed with DIPEL®.

### Example 9. Field Trial with Bacillus thuringiensis Strains NRRL B-67685, NRRL B-67687, and NRRL B-67688

A field trial with cabbage plants exposed to a relatively high, natural infestation of Plutella xylostella (diamondback moth) was conducted. Whole broths of Bacillus thuringiensis strains NRRL B-67685, NRRL B-67687, and NRRL B-67688 were prepared by culturing the strains in a soy-based medium. A whole broth of DIPEL® (Bacillus thuringiensis subsp. kurstaki strain HD1) produced in the soy-based medium as well as the commercially formulated DIPEL® (Bacillus thuringiensis subsp. kurstaki strain HD1) were included for comparison. No symptoms of phytotoxicity in the plants were observed with any of the treatments.

The whole broths were applied as foliar treatments at 750 grams per hectare to plants on July 11 and July 15 at a growth stage of BBCH14 and BBCH16 as outlined in Table 7. The average pest severity in treatment groups and the untreated control group was evaluated on July 13 and July 25. Pest control was calculated as ABBOTT (%):

$$ABBOTT \% = ( \frac{Sample Pest Severity}{Average Pest Severity Untreated} ) \times 100 \%$$

The average percent pest control resulting from each treatment is shown in Table 7. 0% means an efficacy which corresponds to that of the untreated control while an efficacy of 100% means that no damage from the pest infestation was observed.

TABLE 7

| | | | | Relative |
|---|---|---|---|---|
| | | % Pest | % Pest | Zwittermicin |
| | Application | Control on | Control on | Levels from |
| Product | Code | July 13 | July 25 | Example 2 |
| Untreated Control | — | 0 | 0 | |
| B. thuringiensis | AB | 80 | 42 | 81801 |
| NRRL B-67688 | | | | |
| B. thuringiensis | AB | 77 | 46 | 103527 |
| NRRL B-67685 | | | | |
| B. thuringiensis | AB | 74 | 46 | 150730 |
| NRRL B-67687 | | | | |
| B. thuringiensis | AB | 65 | 22 | Not |
| subsp. kurstaki | | | | measured |
| HD1 | | | | |
| DIPEL ® | AB | 58 | 31 | 79184 |
| (commercially | | | | |
| formulated) | | | | |

TABLE 8

| Application Code | Application Date | Growth Stage |
|---|---|---|
| A | July 11 | 14 |
| B | July 15 | 16 |

The results in Table 7 clearly show that the observed insecticidal activities of Bacillus thuringiensis strains NRRL B-67685, NRRL B-67687, and NRRL B-67688 were superior compared to DIPEL® (Bacillus thuringiensis subsp. kurstaki strain HD1) in this field trial.

Unless defined otherwise, all technical and scientific terms herein have the same meaning as commonly understood by one of ordinary skill in the art to which this invention belongs. All publications, patents, and patent publications cited are incorporated by reference herein in their entirety for all purposes.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A composition comprising a biologically pure culture of a Bacillus thuringiensis strain or a cell-free preparation thereof comprising zwittermicin A, Vip3Aa, Cry1Aa, and Cry1Ab,
wherein expression of zwittermicin A with Vip3Aa, Cry1Aa, and/or Cry1Ab results in a synergistic insecticidal effect and wherein the zwittermicin A is present in an amount at least 25-fold greater than that in a biologically pure culture of Bacillus thuringiensis subsp. kurstaki strain EG7841.

2. A composition comprising a biologically pure culture of a Bacillus thuringiensis strain or a cell-free preparation thereof comprising zwittermicin A, Vip3Aa, Cry1Aa, and Cry1Ab,
wherein expression of zwittermicin A with Vip3Aa, Cry1Aa, and/or Cry1Ab1 results in a synergistic insecticidal effect and wherein the zwittermicin A is present in an amount at least 5-fold greater than that in a biologically pure culture of Bacillus thuringiensis subsp. aizawai strain ABTS-1857.

3. The composition of claim 1 further comprising Cry1Ca and Cry1Da, wherein expression of zwittermicin A with Vip3Aa, Cry1Aa, Cry1Ab, Cry1Ca and/or Cry1Da results in a synergistic insecticidal effect.

4. The composition of claim 1, wherein
Vip3Aa is expressed from a gene comprising a DNA sequence exhibiting at least 99% sequence identity to SEQ ID NO: 1;
Cry1Aa is expressed from a gene comprising a DNA sequence exhibiting at least 99% sequence identity to SEQ ID NO: 5; and
Cry1Ab is expressed from a gene comprising a DNA sequence exhibiting at least 99% sequence identity to SEQ ID NO: 6.

5. The composition of claim 3, wherein
Vip3Aa is expressed from a gene comprising a DNA sequence exhibiting at least 99% sequence identity to SEQ ID NO: 1;
Cry1Aa is expressed from a gene comprising a DNA sequence exhibiting at least 99% sequence identity to SEQ ID NO: 5;
Cry1Ab is expressed from a gene comprising a DNA sequence exhibiting at least 99% sequence identity to SEQ ID NO: 6;
Cry1Ca is expressed from a gene comprising a DNA sequence exhibiting at least 99% sequence identity to SEQ ID NO: 16; and
Cry1Da is expressed from a gene comprising a DNA sequence exhibiting at least 99% sequence identity to SEQ ID NO: 18.

6. The composition according to claim 1, wherein the synergistic insecticidal effect results in increased developmental delay and/or mortality.

7. The composition according to claim 1, wherein the synergistic insecticidal effect occurs with Spodoptera exigua H\ddot{υ}bner, Plutella xylostella (L.), and/or Trichoplusia ni (H\ddot{υ}bner).

8. The composition according to claim 1, wherein the Bacillus thuringiensis strain is Bacillus thuringiensis strain NRRL B-67685, Bacillus thuringiensis strain NRRL B-67687, Bacillus thuringiensis strain NRRL B-67688, or an insecticidal mutant thereof having all the identifying characteristics of the respective strain.

9. The composition according to claim 8 comprising a fermentation product of Bacillus thuringiensis strain NRRL B-67685, Bacillus thuringiensis strain NRRL B-67687, Bacillus thuringiensis strain NRRL B-67688, or an insecticidal mutant thereof having all the identifying characteristics of the respective strain.

10. The composition according to claim 8, wherein the insecticidal mutant strain has a genomic sequence with greater than about 90% sequence identity to Bacillus thuringiensis strain NRRL B-67685, Bacillus thuringiensis strain NRRL B-67687, or Bacillus thuringiensis strain NRRL B-67688.

11. The composition according to claim 1, further comprising an agriculturally acceptable carrier, inert, stabilization agent, preservative, nutrient, and/or physical property modifying agent.

12. The composition according to claim 1, wherein the composition is a liquid formulation or a solid formulation.

13. The composition according to claim 12, wherein the composition is a liquid formulation and comprises at least about 1×10⁴ colony forming units (CFU) of the Bacillus thuringiensis strain/mL.

14. A method of controlling an animal pest, comprising applying to the animal pest or an environment thereof an effective amount of the composition according to claim 1.

15. A method of protecting a useful plant or a part of a useful plant in need of protection from animal pest damage, the method comprising contacting an animal pest, a plant, a plant propagule, a seed of a plant, and/or a locus where a plant is growing or is intended to grow with an effective amount of the composition according to claim 1.

16. The method according to claim 14, wherein the composition is applied at about 1×10⁴ to about 1×10¹⁴ CFU per hectare or at about 0.1 kg to about 20 kg fermentation solids per hectare.

17. The method according to claim 16, wherein the animal pest is from the order of Lepidoptera and is Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Cheimatobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, or Trichoplusia spp.

18. The method according to claim 17 wherein the animal pest is Spodoptera exigua, Plutella xylostella, or Trichoplusia ni.

19. The method according to claim 15, wherein the useful plant is selected from the group consisting of soybean, corn, wheat, triticale, barley, oat, rye, rape, millet, rice, sunflower, cotton, sugar beet, pome fruit, stone fruit, citrus, banana, strawberry, blueberry, almond, grape, mango, papaya, peanut, potato, tomato, pepper, cucurbit, cucumber, melon, watermelon, garlic, onion, broccoli, carrot, cabbage, bean, dry bean, canola, pea, lentil, alfalfa, trefoil, clover, flax, elephant grass, grass, lettuce, sugarcane, tea, tobacco and coffee; each in its natural or genetically modified form.

20-21. (canceled)
